(19) United States
(12) Patent Application Publication
(10) Pub. No.: US 2007/0213355 A1
(43) Pub. Date: Sep. 13, 2007

(54) 1H-Imidazo[4,5-C]Quinoline Derivatives in the Treatment of Protein Kinase Dependent Diseases

(76) Inventors: Hans-Georg Capraro, Rheinfelden (CH); Pascal Furet, Thann (FR); Carlos Garcia-Cheverria, Basel (CH); Frederic Stauffer, Reinach (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/579,876

(22) PCT Filed: Nov. 19, 2004
(86) PCT No.: PCT/EP04/13179

§ 371 Date: Feb. 2, 2007

Related U.S. Application Data
(60) Provisional application No. 60/524,214, filed on Nov. 21, 2003.

(51) Int. Cl. A61K 31/437; C07D 471/04

(52) U.S. Cl. 514/293; 546/82

(57) ABSTRACT

The invention relates to the use of imidazoquinolines and salts thereof in the treatment of protein kinase dependent diseases and for the manufacture of pharmaceutical preparations for the treatment of said diseases, imidazoquinolines for use in the treatment of protein kinase dependent diseases, a method of treatment against said diseases, comprising administering the imidazoquinolines to a warm-blooded animal, especially a human, pharmaceutical preparations comprising an imidazoquinoline, especially for the treatment of a protein kinase dependent disease, novel imidazoquinolines, and a process for the preparation of the novel imidazoquinolines.

The invention relates to the use of imidazoquinolines and salts thereof in the treatment of protein kinase dependent diseases and for the manufacture of pharmaceutical preparations for the treatment of said diseases, imidazoquinolines for use in the treatment of protein kinase dependent diseases, a method of treatment against said diseases, comprising administering the imidazoquinolines to a warm-blooded animal, especially a human, pharmaceutical preparations comprising an imidazoquinoline, especially for the treatment of a protein kinase dependent disease, novel imidazoquinolines, and a process for the preparation of the novel imidazoquinolines.

### BACKGROUND OF THE INVENTION

Recently, the concept of treating proliferative diseases by using drugs designed specifically against abnormally active protein kinases has been definitely proven in the treatment of chronic myeloid leukemia (CML) where a first product has now been approved for successful treatment. Clinical studies showed that the drug (N-{5-[4-methyl-piperazino-methyl)-benzoylamido]-2-methylphenyl}-4-(3-pyridyl)-2-pyrimidine-amine, especially in the form of the methane sulfonate (monomesylate) salt called ST1571, which is sold e.g. under the tradename Glivec®/Gleevec®, has impressive activity against chronic phase CML. Typical for CML is a characteristic t(9;22) translocation that juxtaposes the 5′ end of the bcr gene with the 3′ end of the abl gene, resulting in a unique 210 kDa fusion protein p210^{bcr/abl} with constitutive kinase activity. The result is a p210^{bcr/abl}-induced transformation ultimately leading to CML. ST1571 is a reversible inhibitor that occupies the ATP binding pocket of p210^{bcr/abl} and stabilizes the kinase in an inactive conformation. This inhibitory action appears to be the basis for its action against CML.

Over-expression or constitutive expression (activity) of protein kinases appears to be a general principle for transformations that finally lead to proliferative growth of cells and thus cancer, psoriasis or other proliferative diseases.

Protein Kinase B (PKB, also known as Akt) is a member of a conserved family of kinases that includes PKBα, PKBβ, and PKγ in humans. This serine/threonine kinase mediates the physiological effects of several peptide growth factors, including platelet-derived growth factor, insulin, and insulin-like growth factor-I. PKB contains a pleckstrin homology (PH) domain in its amino-terminal domain, a kinase domain in the middle, and a regulatory domain in the carboxy-terminal region. The binding of phosphoinositides to the PH domain of PKB recruits PKB to the plasma membrane where it is phosphorylated on threonine-308/309 and on serine-473. Activation of the PKB pathways results in cellular proliferative, as well as antiapoptotic tumor cell responses. PKBα is amplified in 20% of gastric adenocarcinoma and PKBβ is amplified in 15% of ovarian cancers, 12% of pancreatic cancers, and 3% of breast carcinomas. PKBγ expression and activity is elevated in estrogen receptor negative breast cancer cells and in androgen-independent prostate cancer.

Compounds that down-regulate the kinase activity of PKB may prove to be of clinical Interest for single and combined anticancer treatment modalities.

PDK1 (3-phosphoinosite-dependent protein kinase 1), which is a member of the AGC family of kinases, contributes to the activation of PKB by phosphorylating this protein at Thr-308/309 (the two numbers refer to the different protein isoforms). PDK1 kinase inhibitors could potentially have a therapeutic value by blocking the activation of the PKB mediated signal transduction pathways in cancer and other diseases such as Cowden syndrome, Lhermitte-Dudos disease and Bannayan-Zonana syndrome.

What is desirable from the point of view of possible treatments of proliferative diseases is to have a plethora of compound classes each tailored to specific protein kinases or protein kinase classes, thus allowing to come to specific treatments. Therefore, a strong need exists to find new classes of compounds allowing for such specific inhibitory effects.

### SUMMARY OF THE INVENTION

The class of imidazoquinoline compounds described herein, especially novel compounds falling under this class, has surprisingly been found to have pharmaceutically advantageous properties, inter alia allowing for the inhibition of specific types or classes or groups of protein kinases, especially PDK1, and as inhibitors of lipid kinases, in particular, phosphoinosite 3-kinases, or PI3K or Pi3. The class of imidazoquinoline compounds described herein also show inhibitory activity against KDR, PDGFR, c-Kit, Flt-3 and Flt-4.

The class of Imidazoquinoline compounds described herein further inhibit mutants of said kinases.

In addition to this established activity, the imidazoquinolines have the advantage that their backbone in addition allows for a plethora of substitution patterns that offer a broad possibility to achieve a fine tuning for specific interaction with the ATP binding site of the targeted kinase or kinases, thus opening a new perspective and providing kinase inhibitors of various degrees of specificity.

### DETAILED DESCRIPTION OF THE INVENTION

The invention in particular relates to imidazoquinolines compounds of the formula (I) 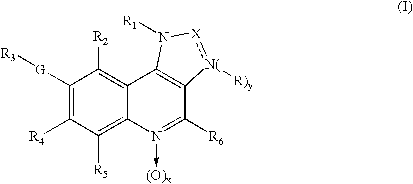
wherein
each of x and y is, independently of the other, 0 or 1;
R₁ is an organic moiety that can, be bound to nitrogen;
X is C═O or C═S with the proviso that then the dashed line bonding X to N is absent, so that X is bound to the adjacent N via a single bond the with the proviso that then y is 1 and R is hydrogen or an organic moiety that can be bound to nitrogen;
or X is (CR₇) wherein R₇ is hydrogen or an organic or inorganic moiety with the proviso that then the dashed line bonding X to N is a bond, so that X is bound to the adjacent N via a double bond, and with the proviso that then y is zero or y is 1 and then —R is →O;
G is unsubstituted or substituted alkenylene, unsubstituted or substituted alkynylene; and
each of R₂, R₃, R₄, R₅ and R₆ independently of the others, is hydrogen, an organic moiety or an inorganic moiety;
   or pharmaceutically acceptable salts thereof,
   and use of compounds of formula (I) in the treatment of protein kinase dependent diseases or for the manufacture of pharmaceutical preparations for the treatment of protein kinase dependent diseases.

The present invention also relates to a method of treating protein kinase dependent diseases comprising administering imidazoquinoline compounds of the formula (I) to a warm-blooded animal, especially a human. The present invention also relates to pharmaceutical preparations comprising an imidazoquinoline compound of the formula (I), especially for the treatment of a protein kinase dependent disease, novel imidazoquinoline compounds of the formula (I), a process for the manufacture of the novel imidazoquinoline compounds of the formula (I), and novel starting materials and intermediates for their manufacture. The present invention also relates to use of a compound of formula (I) in the manufacture of a pharmaceutical preparation for the treatment of a protein kinase dependent disease.

The general terms used hereinbefore and hereinafter preferably have within the context of this disclosure the following meanings, unless otherwise indicated:

The prefix “lower” denotes a radical having 1 up to and including a maximum of 7, especially 1 up to and including a maximum of 4 carbon atoms, the radicals in question being either linear or branched with single or multiple branching. Lower alkyl, for example, is methyl, ethyl, n-propyl, sec-propyl, n-butyl, isobutyl, sec-butyl, tert-butyl, n-pentyl, n-hexyl or n-heptyl.

An organic moiety that can be bound to nitrogen is preferably unsubstituted or substituted alkyl, unsubstituted or substituted alkenyl, unsubstituted or substituted alkynyl, unsubstituted or substituted aryl, unsubstituted or substituted aryl-lower alkyl or aryl-lower alkoxy, unsubstituted or substituted heterocyclyl, unsubstituted or substituted heterocyclyl lower alkyl or lower alkoxy, unsubstituted or substituted cycloalkyl or unsubstituted or substituted cycloalkenyl.

An organic moiety is preferably unsubstituted or substituted alkyl, unsubstituted or substituted alkenyl, unsubstituted or substituted alkynyl, unsubstituted or substituted unsubstituted or substituted aryl, unsubstituted or substituted heterocyclyl, unsubstituted or substituted cycloalkyl or unsubstituted or substituted cycloalkenyl, unsubstituted or substituted arylcarbonylamino, amino substituted by one or two moieties selected from the group consisting of lower alkyl, substituted lower alkyl moieties, aryl, cycloalkyl and mercapto-lower alkyl, alkyloxy or cyano.

Halo or halogen is preferably fluoro, chloro, bromo or iodo, most preferably fluoro, chloro or bromo.

Alkyl preferably has up to 20, more preferably up to 12 carbon atoms and is linear or branched one or more times; preferred is lower alkyl, especially C₁-C₄alkyl. Alkyl may be linear or cyclic and can be unsubstituted or substituted, preferably by one or more substituents independently selected from those mentioned below under “substituted”. Unsubstituted alkyl, preferably lower alkyl, or hydroxyalkyl, especially hydroxy-lower alky, e.g. 2-hydroxyethyl or cyclo-lower alky, e.g. cyclopropyl, is especially preferred as an organic moiety that can be bound to nitrogen.

Among the moieties corresponding to substituted alkyl, unsubstituted or substituted aryl-lower alkyl (especially preferred), heterocyclyl-lower alkyl, or cycloalkyl-lower alkyl are also preferred.

Aryl-lower alkyl is preferably lower alkyl that is substituted (preferably terminally or in 1-position) by unsubstituted or substituted aryl as defined below, especially phenyl-lower alkyl, such as benzyl or phenylethyl, especially 1-phenylethyl.

Heterocyclyl-lower alkyl is preferably lower alkyl that is substituted (preferably terminally) by unsubstituted or substituted heterocyclyl as defined below.

Cycloalkyl-lower alkyl is preferably lower alkyl that is substituted (preferably terminally) by unsubstituted or substituted cycloalkyl as defined below.

Alkenyl is preferably a moiety with one or more double bonds and preferably has 2-20, more preferably up to 12, carbon atoms; it is linear or branched one or more times (as far as possible in view of the number of carbon atoms). Preferred is C₂-C₇alkenyl, especially C₃-C₄alkenyl, such as allyl or crotyl. Alkenyl can be unsubstituted or substituted, especially by one or more, more especially up to three, of the substituents mentioned below under “substituted”. Substituents such as amino or hydroxy (with free dissociable hydrogen) preferably are not bound to carbon atoms that participate at a double bond, and also other substituents that are not sufficiently stable are preferably excluded. Unsubstituted alkenyl, in particular C₂-C₇alkenyl, is preferred.

When G is alkenylene, C₂-C₇alkenylene is preferred, with ethenylene (—C═C—) most preferred. When G is alkynylene, C₂-C₇alkynylene is preferred, with ethynylene (—C≡C—) most preferred.

Alkynyl is preferably a moiety with one or more triple bonds and preferably has 2-20, more preferably up to 12, carbon atoms; it is linear of branched one or more times (as far as possible in view of the number of carbon atoms). Preferred is C₂-C₇alkynyl, especially C₃-C₄alkynyl, such as ethynyl or propyn-2-yl. Alkynyl can be unsubstituted or substituted, especially by one or more, more especially up to three, of the substituents mentioned below under “substituted”. Substituents such as amino or hydroxy (with free dissociable hydrogen) preferably are not bound to carbon atoms that participate at a triple bond, and also other substituents that are not sufficiently stable are preferably excluded. Unsubstituted alkynyl, in particular C₂-C₇alkynyl, is preferred.

Aryl preferably has a ring system of not more than 20 carbon atoms, especially not more than 16 carbon atoms, is preferably mono-, bi- or tric-cyclic, and is unsubstituted or substituted preferably as defined below under “substituted”. For example, aryl is selected from phenyl, naphthyl, indenyl, azulenyl and anthryl, and is preferably in each case unsubstituted or halo (especially fluoro, chloro, bromo or iodo); halo-lower alkyl (especially trifluoromethyl); sulfonamide (NH₂—S(O)₂—); dioxolo; hydroxy; amino; lower alkoxy (especially methoxy); hydroxy-lower alkyl (especially hydroxymethyl or 2-hydroxyethyl); mono or disubstituted amino; cyclic amino; amino-lower alkyl (especially aminomethyl, 2-aminoethyl or 3-aminopropyl); lower alkyl (especially methyl or ethyl); cyano; cyano-lower alkyl (especially 2-cyanoethyl); amidino; N-hydroxyamidino; amidino-lower alkyl (especially 2-amidino-ethyl); N-hydroxyamidino-lower alkyl (especially 2-(N-hydroxyamidino)-ethyl) substituted phenyl; or (especially 1- or 2-) naphthyl. Unsubstituted or substituted aryl, preferably phenyl; hydroxyphenyl (such as 4-hydroxyphenyl); methoxyphenyl (such as 2-, 3- or 4-methoxyphenyl); benzo[1,3]dioxolo; lower alkyl (such methyl or ethyl); is especially preferred as organic moiety that can be bound to nitrogen or as organic moiety R₂ to R₇.

In arylcarbonylamino, aryl is preferably aryl as defined in the last paragraph, especially benzoylamino.

Heterocyclyl is preferably a heterocyclic radical that is unsaturated, saturated or partially saturated in the bonding ring and is preferably a monocyclic or in a broader aspect of the invention bi- or tri-cyclic ring; has 3-24, more preferably 416 ring atoms; wherein at least in the ring bonding to the radical of the molecule of formula (I) one or more, preferably one to four, especially one or two carbon ring atoms are replaced by a heteroatom selected from the group consisting of nitrogen, oxygen and sulfur, the bonding ring preferably having 4-12, especially 4-7 ring atoms; heteroaryl being unsubstituted or substituted by one or more, especially 1-4, substituents independently selected from the group consisting of the substituents defined below under “substituted”; especially being a heterocyclic radical selected from the group consisting of oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, pyranyol, thiazolyl, isothiazolyl, dithiazolyl, oxazolyl, isoxazolyl, pyridyl, pyridinyl, pyrazinyl, pyrimidinyl, piperidyl, piperazinyl, pyridazinyl, morpholinyl, thiomorpholinyl, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, chromenyl, isochromanyl and chromanyl, each of these radicals being unsubstituted or substituted by one to two radicals selected from the group consisting of oxy, lower alkyl, especially methyl or tert-butyl, lower alkoxy, especially methoxy, and halo, especially fluoro or chloro. Unsubstituted or substituted heterocyclyl (e.g. morpholinyl, piperazinyl, lower alkyl piperazinyl, piperidino, piperidyl, pyrrolidinyl and azetidinyl) are preferred.

Cycloalkyl is preferably C₃-C₁₀cycloalkyl, especially cyclopropyl, dimethylcyclopropyl, cyclobutyl, cyclopentyl, cyclohexyl or cycloheptyl, cycloalkyl being unsubstituted or substituted by one or more, especially 1-3, substituents independently selected from the group consisting of the substituents defined below under “substituted”.

Cycloalkenyl is preferably C₅-C₁₀cycloalkenyl, especially cyclopentenyl, cyclohexenyl or cycloheptenyl, cycloalkenyl being unsubstituted or substituted by one or more, especially 1-3, substituents independently selected from the group consisting of the substituents defined below under “substituted”.

An inorganic moiety R₂ to R₇ is preferably halogen, especially fluoro, chloro, bromo or iodo, hydroxy, amino, cyano or nitro.

An organic moiety R₂ to R₇ is selected from the organic moieties mentioned above for organic moieties that can be bound to nitrogen (for R₁) or is alternatively selected from the group consisting of unsubstituted or substituted alkoxy (e.g. lower alkoxy) or phenyl-lower alkoxy (e.g. methoxy); or lower alkanoyloxy (e.g. acetoxy); amino substituted by one or two moieties selected from the group consisting of lower alkyl (e.g. methyl, n-butyl, cyclopropyl or isopropyl); hydroxy-lower alkyl (e.g. 2-hydroxyethyl); mercapto-lower alkyl (e.g. 2-mercaptoethyl); unsubstituted or substituted C₅-C₁₄aryl, as defined above (e.g. phenyl, hydroxyphenyl, methoxyphenyl or aminosulfonyl-phenyl or benzo[1,3]dioxolo); a heteroaryl being unsubstituted or substituted by one or more, especially 1-3, substituents independently selected from the group consisting of the substituents defined below under “substituted”; especially being pyridyl (or an N-oxide of pyridyl) which is unsubstituted or substituted by one to two radicals selected from the group consisting of lower alkyl (e.g. methyl); lower alkoxy (e.g. methoxy); halo (e.g. fluoro); or —NR₈R₉, wherein R₈ and R₉ can be the same or different and are independently H; lower alkyl (e.g. methyl, ethyl or propyl); lower cycloalkyl (e.g. cyclopropyl) or the R₈ and R₉ can, with the N atom, form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl); cycloalkyl as defined above, especially C₃-C₈cycloalkyl; lower alkanoyl (preferably as single amino substituent or in combination with another of the non-acyl moiety just mentioned) and benzoyl or phenyl-lower alkanoyl (preferably as single amino substituent or in combination with another of the non-acyl moiety just mentioned); cyano; cyano-lower alkyl (such as cyanomethyl); amidino; N-hydroxyamidino; amidino-lower alkyl (such as -methyl); or N-hydroxyamidino-lower alkyl (such as -methyl).

Preferably, only up to five, more preferably up to two of R₂, R₃, R₄, R₅, R₆ and R₇ are/is other than hydrogen (that is, an inorganic or organic moiety).

A very preferred group of compounds of formula (I) are those wherein R₃ is one of the organic moieties other than hydrogen, especially those mentioned as being preferred above.

“Substituted”, wherever used for a moiety, means that one or more hydrogen atoms in the respective moiety, especially up to five, more especially up to three, of the hydrogen atoms are replaced independently of each other by the corresponding number of substituents which preferably are independently selected from the group consisting of lower alkyl (e.g. methyl, ethyl or propyl); halo (e.g. F, Cl, Br or I); halo-lower alkyl (e.g. trifluoromethyl); hydroxy; carboxy; lower alkoxy (e.g. methoxy); phenyl-lower alkoxy; lower alkanoyloxy; lower alkanoyl; hydroxy-lower alkyl (e.g. hydroxymethyl or 2-hydroxyethyl); amino; mono or disubstituted amino; cyclic amino; amino-lower alkyl (e.g. aminomethyl, 2-aminoethyl or 3-aminopropyl); N-lower alkylamino; N,N-di-lower alkylamino; N-lower alkyl amino alkyl (e.g. methyl aminoethyl, cyclopropyl aminoethyl); N,N-di-lower alkyl amino alkyl; N-phenyl-lower alkylamino; N,N-bis(phenyl-lower alkyl)-amino; amino lower alkoxy (e.g. methoxy amino and methoxy N-methylamino); lower alkanoylamino; benzoylamino; carbamoyl-lower alkoxy; N-lower alkylcarbamoyl-lower alkoxy or N,N-di-lower alkylcarbamoyl-lower alkoxy; amidino; N-hydroxy-amidino; guanidine; amidino-lower alkyl (e.g. 2-amidinoethyl); N-hydroxyamidino-lower alkyl (e.g. N-hydroxy-amidino-methyl or -2-ethyl); carboxy; lower alkoxycarbonyl; phenyl; naphthyl; fluorenyl-lower alkoxycarbonyl (e.g. benzyloxycarbonyl); lower alkanoyl; sulfo; lower alkanesulfonyl (e.g. methanesulfonyl (CH₃—S(O)₂—)); sulfonamide (NH₂—S(O)₂—); N-lower alkyl sulfonamide alkyl (e.g. CH₃—NH₂—S(O)₂-alkyl); dioxolo; phosphono (—P(═O)(OH)₂); hydroxy-lower alkoxy phosphoryl or di-lower alkoxyphosphoryl; carbamoyl; mono- or di-lower alkylcarbamoyl; sulfamoyl; sulfamide; mono- or di-lower alkylaminosulfonyl; cyano-lower alkyl (e.g. cyanomethyl); C₅-C₁₆aryl (e.g. phenyl or naphthyl) where C₅-C₁₆aryl is substituted with any of the substituents defined above, and especially is phenyl which is unsubstituted or substituted with up to four, preferably up to three substituents, wherein the substituents are the same or different and are independently selected from halo (e.g. Cl or F); cyano; cyano lower alkyl (e.g. cyanomethyl, cyanoethyl and cyanopropyl); lower alkyl; lower alkoxy; amino-lower alkyl; N-lower alkyl amino alkyl (e.g. methyl aminoethyl, cyclopropyl aminoethyl); N,N-di-lower alkyl amino alkyl; amino-lower alkoxy; azetidinyl lower alkyl; pyrrolidinyl; amino-lower alkyl sulfanyl or thiol-lower alkyl; wherein the amino group can be mono or disubstituted [e.g. —(C₁-C₇)NR₈R₉ or —O—(C₁-C₇)NR₈R₉, wherein R₈ and R₉ can be the same or different and are independently H, lower alkyl (e.g. methyl, ethyl or propyl), lower cycloalkyl (e.g. cyclopropyl) or R₈ and R₉ together with the N atom form a 3 to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl)].

“Substituted” also includes: amino-carbonyl-lower alkyl (e.g. R₈R₉—N—C(O)—CH₂—, wherein R₈ and R₉ are as defined above); heterocyclyl; amine heterocyclyl; heterocyclyl-lower alkyl; heterocyclyl-lower alkoxy or heterocyclyl-lower alkanesulfanyl; wherein the heterocyclyl is a 3- to 8-membered heterocyclic ring containing 1-4 nitrogen, oxygen or sulfur atoms (e.g. imidazolyl, imidazolinyl, pyrrolidinyl, morpholinyl, azetidinyl, pyridyl, piperidino, piperidyl, piperidinyl, piperazinyl, lower alkyl-piperazinyl, lower alkyl piperazinyl-lower alkyl, and substituted heterocyclyls such as pyrrolidin-2-one, oxazolidin-2-one, pyrrolidine-2,5-dione, piperazine-2-one and oxo-oxazolidinyl); C₃-C₁₀cycloalkyl (e.g. cyclopropyl or cyclohexyl); hydroxy-C₃-C₈cycloalkyl (e.g. hydroxy-cyclohexyl); heteroaryl with 5 or 6 ring atoms and 1-4 ring heteroatoms selected from O, N and S, especially furyl and pyridyl; or —NR₈R₉, wherein R₈ and R₉ can be the same or different and are independently H, lower alkyl (e.g. methyl, ethyl or propyl); lower cycloalkyl (e.g. cyclopropyl) or the R₈ and R₉ can, with the N atom, form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl). It goes without saying that substituents are only at positions where they are chemically possible, the person skilled in the art being able to decide (either experimentally or theoretically) without inappropriate effort which substitutions are possible and which are not. For example, amino or hydroxy groups with free hydrogen may be unstable if bound to carbon atoms with unsaturated (e.g. olefinic) bonds.

Salts are preferably the pharmaceutically acceptable salts of compounds of formula (I) if they are carrying salt-forming groups.

Salt-forming groups in a compound of formula (I) are groups or radicals having basic or acidic properties. Compounds having at least one basic group or at least one basic radical, for example, amino, a secondary amino group not forming a peptide bond or a pyridyl radical, may form acid addition salts, for example, with inorganic acids, such as hydrochloric acid, sulfuric acid or a phosphoric acid, or with suitable organic carboxylic or sulfonic acids, for example, aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids, such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethanesulfonic acid, or aromatic sulfonic acids, for example, benzene-, p-toluene- or naphthalene-2-sulfonic acid. When several basic groups are present mono- or poly-acid addition salts may be formed.

Compounds of formula (I) having acidic groups, a carboxy group or a phenolic hydroxy group, may form metal or ammonium salts, such as alkali metal or alkaline earth metal salts, for example, sodium, potassium, magnesium or calcium salts, or ammonium salts with ammonia or suitable organic amines, such as tertiary monoamines, for example, triethylamine or tri-(2-hydroxyethyl)-amine, or heterocyclic bases, for example, N-ethyl-piperidine or N,N′-dimethylpiperazine. Mixtures of salts are possible.

Compounds of formula (I) having both acidic and basic groups can form internal salts.

For the purposes of isolation or purification, as well as in the case of compounds that are used further as intermediates, it is also possible to use pharmaceutically unacceptable salts, e.g. the picrates. Only pharmaceutically acceptable, non-toxic salts may be used for therapeutic purposes, however, and those salts are therefore preferred.

Owing to the close relationship between the novel compounds in free form and in the form of their salts, including those salts that can be used as intermediates, for example, in the purification of the novel compounds or for the identification thereof, any reference hereinbefore and hereinafter to the free compounds shall be understood as including the corresponding salts, where appropriate and expedient.

Where the plural form is used for compounds, salts, pharmaceutical preparations, diseases and the like, this is intended to mean also a single compound, salt, or the like.

Any asymmetric carbon atom may be present in the (R)-, (S)- or (R,S)-configuration, preferably in the (R)- or (S)-configuration. Substituents at a double bond or a ring may be present in cis-(=Z-) or trans (=E-) form. The compounds may thus be present as mixtures of isomers or preferably as pure isomers, preferably as enantiomer-pure diastereomers or pure enantiomers.

The present invention also relates to pro-drugs of a compound of formula (I) that convert in vivo to the compound of formula (I) as such. Any reference to a compound of formula (I) is therefore to be understood as referring also to the corresponding pro-drugs of the compound of formula (I), as appropriate and expedient.

The terms “treatment” or “therapy” refer to the prophylacetic or preferably therapeutic (including but not limited to palliative, curing, symptom-alleviating, symptom-reducing, kinase-regulating and/or kinase-inhibiting) treatment of said diseases, especially of the diseases mentioned below.

Where subsequently or above the term “use” is mentioned (as verb or noun) (relating to the use of a compound of the formula (I) or a pharmaceutically acceptable salt thereof), this includes any one or more of the following embodiments of the invention, respectively: the use in the treatment of a protein kinase dependent disease, the use for the manufacture of pharmaceutical compositions for use in the treatment of a protein kinase dependent disease, methods of use of one or more compounds of the formula (I) in the treatment of a protein kinase dependent disease, the use of pharmaceutical preparations comprising one or more compounds of the formula (I) for the treatment of a protein kinase dependent disease, and one or more compounds of the formula (I) for use in the treatment of a protein kinase dependent disease, as appropriate and expedient and if not stated otherwise. In particular, diseases to be treated and are thus preferred for “use” of a compound of formula (I) are selected from protein kinase dependent (“dependent” meaning also “supported”, not only “solely dependent”) diseases mentioned herein, especially proliferative diseases mentioned herein, more especially any one or more of these or other diseases that depend on one or more of PDK1 or PI3K, or any combinations of these, or a mutant of any one or more of these, and a compound of the formula (I) can therefore be used in the treatment of a kinase dependent disease, especially a disease depending on one or more of the kinases mentioned above and below, where (especially in the case of aberrantly highly-expressed, constitutively activated and/or mutated kinases) said kinase-dependent disease is dependent on the activity of one or more of the said kinases or the pathways they are involved.

The compounds of formula (I) have valuable pharmacological properties and are useful in the treatment of protein kinase dependent diseases, for example, as drugs to treat proliferative diseases.

### Preferred Embodiments of the Invention

With the groups of preferred compounds of formula (I) mentioned hereinafter, definitions of substituents from the general definitions mentioned hereinbefore may reasonably be used, for example, to replace more general definitions with more specific definitions or especially with definitions characterized as being preferred.

The invention relates especially to a compound of the formula (I),

wherein

each of x and y is, independently of the other, 0 or 1;
R₁ is an organic moiety that can be bound to nitrogen;
X is C═O or C═S with the proviso that then the dashed line bonding X to N is absent, so that X is bound to the adjacent N via a single bond and with the proviso that then y is 1 and R is hydrogen or an organic moiety that can be bound to nitrogen; or
X is (CR₇), wherein R₇ is hydrogen or an organic or inorganic moiety with the proviso that then the dashed line bonding X to N is a bond, so that X is bound to the adjacent N via a double bond, and with the proviso that then y is zero or y is 1 and then —R is →O;
G is unsubstituted or substituted alkenylene, unsubstituted or substituted alkynylene; and
each of R₂, R₃, R₄, R₅ and R₆, independently of the others, is an organic moiety or hydrogen or an Inorganic moiety;
   or a pharmaceutically acceptable salt thereof,
   and its use in the treatment of a protein kinase dependent disease or for the manufacture of a pharmaceutical preparation for the treatment of a protein kinase dependent disease, or a method of treatment against said disease comprising administering a compound of the formula (I) to a warm-blooded animal, especially a human, in need of such treatment.

A tyrosine kinase dependent disease is preferably one depending on PDK1, PI3K and especially (aberrantly highly-expressed or activated) PKB/Akt (=PKB)-dependent disease or disease dependent on the activation of the PI3K/PKB pathway. The class of imidazoquinoline compounds described herein also show inhibitory activity against KDR, PDGFR, c-Kit, Flt-3 and Flt-4.

Also preferred is a compound of the formula (I), or a pharmaceutically acceptable salt thereof, for use in the treatment of, or preparation of a pharmaceutical composition for the treatment of, a protein kinase dependent disease, especially one depending on PDK1, PI3K and (especially aberrantly highly expressed or activated) PKB/Akt (=PKB)-dependent disease or disease dependent on the activation of the PI3K/PKB pathway.

Especially preferred is a compound of the formula (I), or a pharmaceutically acceptable salt thereof, wherein X is C═O or CR₇ and the other moieties are as defined under formula (I), for use in the diagnostic or therapeutic treatment of a warm-blooded animal, especially a human.

More preferred is a compound of formula (I), or a pharmaceutically acceptable salt thereof, wherein
each of x and y is, independently of the other, 0 or 1;
R₁ is substituted or unsubstituted aryl or heteroaryl especially phenyl, where the phenyl is substituted with up to 4, preferably up to 2 substituents, wherein the substituents are the same or different and are independently selected from: halo; cyano; cyano lower alkyl; lower alkyl; lower alkoxy; amino; amino-lower alkyl; amino-lower alkoxy; amino-lower alkyl sulfanyl or thiol-lower alkyl; wherein the amino group can be mono or disubstituted [e.g. —(C₁-C₇)NR₈R⁹ or —O—(C₁-C₇)NR₈R₉, wherein R₈ and R₉ can be the same or different and are independently H, lower alkyl, lower cycloalkyl or R₈ and R₉ together with the N atom form a 3- to 8-membered heterocyclic ring containing 1-4 nitrogen, oxygen or sulfur atoms]; amino-carbonyl-lower alkyl; heterocyclyl; heterocyclyl-lower alkyl; heterocyclyl-lower alkoxy or heterocyclyl-lower alkanesulfanyl wherein the heterocyclyl is a 3- to 8-membered heterocyclic ring containing 1-4 nitrogen, oxygen or sulfur atoms; wherein alkyl may be linear or cyclic and the alkyl in any of the substituents above may optionally be substituted with —NR₈R₉, wherein R₈ and R₉ are as defined above;
X is C═O or C═S with the proviso that then the dashed line bonding X to N is absent, so that X is bound to the adjacent N via a single bond and with the proviso that then y is 1 and R is hydrogen or an organic moiety that can be bound to nitrogen; or
X is (CR₇), wherein R₇ is hydrogen or an organic moiety, such as C₁-C₇lower alkyl; amino or amino-lower-alkyl; wherein the alkyl may be unsubstituted or substituted with halo, lower alkoxy, or cycloalkyl with the proviso that then the dashed line bonding X to N is a bond, so that X is bound to the adjacent N via a double bond, and with the proviso that then y is zero, or y is 1 and then —R is →O;
G is unsubstituted or substituted alkenylene; unsubstituted or substituted alkynylene; R₂ is hydrogen;
R₃ is hydrogen lower alkyl; halo; lower alkoxy; unsubstituted or substituted C₅-C₁₄aryl; or a heteroaryl being unsubstituted or substituted by one or more, especially 1-4 substituents independently selected from the group consisting of the substituents defined above under unsubstituted; especially being pyridyl (or an N-oxide of pyridyl) which is unsubstituted or substituted by one to two radicals selected from the group consisting of lower alkyl, lower alkoxy, halo, or —NR₈R₉, wherein R₈ and R₉ can be the same or different and are independently H, lower alkyl (e.g. methyl, ethyl or propyl), lower cycloalkyl or the R₈ and R₉ can, with the N atom, form a 3- to 8-membered heterocyclic ring containing 1-4 nitrogen, oxygen or sulfur atoms;
R₄ is hydrogen or halo;
R₅ is hydrogen; and
R₆ is hydrogen, amino, amino-lower alkyl or alkylamido;
   or a pharmaceutically acceptable salt thereof as such, especially for use in the preparation of a pharmaceutical composition, or for use in the diagnostic or therapeutic treatment of a warm-blooded animal, especially a human.

Especially preferred is a compound of formula (I),

wherein

each of x and y is, independently of the other, 0 or 1;
R₁ is substituted or unsubstituted phenyl where the phenyl is substituted with up to 4, preferably up to 2 substituents, wherein the substituents are the same or different and are independently selected from halo (e.g. Cl or F); cyano; cyano lower alkyl (e.g. cyanomethyl, cyanoethyl and cyanopropyl); lower alkyl; lower alkoxy; amino; amino-lower alkyl; amino-lower alkoxy; amino-lower alkyl sulfanyl or thiol-lower alkyl; wherein the amino group can be mono or disubstituted, [e.g. —(C₁-C₇)NR₈R₉ or —O—(C₁-C₇)NR₈R₉, wherein R₈ and R₉ can be the same or different and are independently H, lower alkyl (e.g. methyl, ethyl or propyl), lower cycloalkyl (e.g. cyclopropyl) or R₈ and R₉ together with the N atom form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl)]; amino-carbonyl-lower alkyl (e.g. R₈R₉—N—C(O)CH₂, wherein R₈ and R₉ are as defined above); heterocyclyl; heterocyclyl-lower alkyl; heterocyclyl-lower alkoxy or heterocyclyl-lower alkanesulfanyl wherein the heterocyclyl is a 3- to 8-membered heterocyclic ring containing 1-4 nitrogen, oxygen or sulfur atoms (e.g. imidazolyl, imidazolinyl, pyrrolidinyl, morpholinyl, azetidinyl, pyridyl, piperidino, piperidyl, piperazinyl or lower alkyl-piperazinyl); wherein alkyl may be linear or cyclic (e.g. cyclopropyl) and the alkyl in any of the substituents above may optionally be substituted with —NR₈R₉, wherein R₈ and R₉ are as defined above;
X is C═O or C═S with the proviso that then the dashed line bonding X to N is absent, so that X is bound to the adjacent N via a single bond and with the proviso that then y is 1 and R is hydrogen or an organic moiety that can be bound to nitrogen; or
X is (CR₇), wherein R₇ is hydrogen or an organic moiety, such as C₁-C₇lower alkyl; amino; amino-lower alkyl; wherein the alkyl may be unsubstituted or substituted with halo (e.g. methyl, ethyl, propyl, trifluoromethyl); lower alkoxy (e.g. methoxy); or cycloalkyl (e.g. cyclopropyl) with the proviso that then the dashed line bonding X to N is a bond, so that X is bound to the adjacent N via a double bond, and with the proviso that then y is zero, or y is 1 and then —R is →O;
G is unsubstituted or substituted alkenylene (e.g. ethenylene), unsubstituted or substituted alkynylene (e.g. ethynylene);
R₂ is hydrogen;
R₃ is hydrogen; lower alkyl; halo; (e.g. fluoro, chloro or bromo); lower alkoxy (e.g. methoxy) or unsubstituted or substituted C₅-C₁₄aryl, (e.g. phenyl, hydroxyphenyl, methoxyphenyl or aminosulfonyl-phenyl or benzo[1,3]dioxolo); or a heteroaryl being unsubstituted or substituted by one or more, especially 1-4, substituents independently selected from the group consisting of the substituents defined above under “substituted”; especially being pyridyl (or an N-oxide of pyridyl) which is unsubstituted or substituted by one to two radicals selected from the group consisting of lower alkyl (e.g. methyl); lower alkoxy (e.g. methoxy); halo (e.g. fluoro); or —NR₈R₉, wherein R₈ and R₉ can be the same or different and are independently H, lower alkyl (e.g. methyl, ethyl or propyl); lower cycloalkyl (e.g. cyclopropyl); or the R₈ and R₉ can, with the N atom, form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl);
R₄ is hydrogen or halo, (e.g. F or Cl);
R₅ is hydrogen; and
R₆ is hydrogen; amino; amino-lower alkyl or alkylamido (e.g. methylamido —NHC(O)—CH₃); or a pharmaceutically acceptable salt thereof as such, especially for use in the preparation of a pharmaceutical composition, or for use in the diagnostic or therapeutic treatment of a warm-blooded animal, especially a human.

Most especially preferred is a compound of formula (I),

wherein

each of x and y is, independently of the other, 0 or 1;
R₁ is substituted or unsubstituted phenyl where the phenyl is substituted with up to 4, preferably up to 2 substituents, wherein the substituents are the same or different and are independently selected from halo (e.g. Cl or F); cyano; cyano lower alkyl (e.g. cyanomethyl, cyanoethyl and cyanopropyl); lower alkyl; lower alkoxy; N-lower alkyl amino alkyl (e.g. methyl aminoethyl, cyclopropyl aminoethyl); N,N-di-lower alkyl amino alkyl; methoxy amino; methoxy N-methyl amino; amino; amino-lower alkyl; amino-lower alkoxy; azetidinyl lower alkyl; pyrrolidinyl; N-lower alkyl sulfonamide alkyl (e.g. CH₃—NH₂—S(O)₂-alkyl); amino-lower alkyl sulfanyl or thiol-lower alkyl; wherein the amino group can be mono or disubstituted, [e.g. —(C₁-C₇)NR₈R₉ or —O—(C₁-C₇)NR₈R₉, wherein R₈ and R₉ can be the same or different and are independently H, lower alkyl (e.g. methyl, ethyl or propyl), lower cycloalkyl (e.g. cyclopropyl) or R₈ and R₉ together with the N atom form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl)]; amino-carbonyl-lower alkyl (e.g. R₈R₉—N—C(O)—CH₂—, wherein R₈ and R₉ are as defined above); heterocyclyl; heterocyclyl-lower alkyl; lower alkyl piperazinyl-lower alkyl; heterocyclyl-lower alkoxy or heterocyclyl-lower alkanesulfanyl wherein the heterocyclyl is a 3- to 8-membered heterocyclic ring containing 1-4 nitrogen, oxygen or sulfur atoms (e.g. imidazolyl, imidazolinyl, pyrrolidinyl, morpholinyl, azetidinyl, pyridyl, piperidino, piperidyl, piperazinyl or lower alkyl-piperazinyl); substituted heterocyclyls such as pyrrolidin-2-one, oxazolidin-2-one, pyrrolidine-2,5-dione, piperazine-2-one and oxo-oxazolidinyl; wherein alkyl may be linear or cyclic (e.g. cyclopropyl) and the alkyl in any of the substituents above may optionally be substituted with —NR₈R₉, wherein R₈ and R₉ are as defined above;
X is C═O or C═S with the proviso that then the dashed line bonding X to N is absent, so that X is bound to the adjacent N via a single bond and with the proviso that then y is 1 and R is hydrogen or an organic moiety that can be bound to nitrogen; or
X is (CR₇), wherein R₇ is hydrogen or an organic moiety, such as C₁-C₇lower alkyl; amino; amino-lower alkyl; wherein the alkyl may be unsubstituted or substituted with halo (e.g. methyl, ethyl, propyl, trifluoromethyl); lower alkoxy (e.g. methoxy); or cycloalkyl (e.g. cyclopropyl) with the proviso that then the dashed line bonding X to N is a bond, so that X is bound to the adjacent N via a double bond, and with the proviso that then y is zero, or y is 1 and then —R is →O;
G is unsubstituted or substituted alkenylene (e.g. ethenylene), unsubstituted or substituted alkynylene (e.g. ethynylene);
R₂ is hydrogen;
R₃ is hydrogen; lower alkyl; halo (e.g. fluoro, chloro or bromo); lower alkoxy (e.g. methoxy); unsubstituted or substituted C₅-C₁₄aryl (e.g. phenyl, hydroxyphenyl, methoxyphenyl or aminosulfonyl-phenyl or benzo[1,3]dioxolo); or a heteroaryl being unsubstituted or substituted by one or more, especially 1-4, substituents independently selected from the group consisting of the substituents defined above under “substituted”; especially being pyridyl (or an N-oxide of pyridyl) which is unsubstituted or substituted by one to two radicals selected from the group consisting of lower alkyl (e.g. methyl); lower alkoxy (e.g. methoxy); halo (e.g. fluoro); or —NR₈R₉, wherein R₈ and R₉ can be the same or different and are independently H, lower alkyl (e.g. methyl, ethyl or propyl); lower cycloalkyl (e.g. cyclopropyl) or the R₈ and R₉ can, with the N atom, form a 3 to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl);
R₄ is hydrogen or halo, (e.g. F or Cl);
R₅ is hydrogen; and
R₆ is hydrogen; amino; amino-lower alkyl or alkylamido (e.g. methylamido —NHC(O)—CH₃);
   or a pharmaceutically acceptable salt thereof as such,
   especially for use in the preparation of a pharmaceutical composition, or for use in the diagnostic or therapeutic treatment of a warm-blooded animal, especially a human. Especially preferred is a compound of formula (I) for use in the treatment of a protein kinase dependent disease or for the manufacture of a pharmaceutical preparation for the treatment of a protein kinase dependent disease, or a method of treatment against said disease, comprising administering a compound of the formula (I) to a warm-blooded animal, especially a human, in need of such treatment.

Especially preferred is a compound of formula (I) for use in the treatment of a proliferative disease selected from a benign or malignant tumor, carcinoma of the brain, kidney, liver, adrenal gland, bladder, breast, stomach, gastric tumors, ovaries, colon, rectum, prostate, pancreas, lung, vagina or thyroid, sarcoma, glioblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, a mammary carcinoma or a leukemia. Other diseases include Cowden syndrome, Lhermitte-Dudos disease and Bannayan-Zonana syndrome.

Having regard to their inhibition of phosphatidylinositol 3-kinase enzymes, compounds of formula (I) in free or pharmaceutically acceptable salt form, are useful in the treatment of conditions which are mediated by the activation of the PI3K kinase enzymes, particularly inflammatory or allergic conditions. Treatment in accordance with the invention may be symptomatic or prophylacetic. Other preferred embodiments Include pharmaceutical composition comprising a compound according to formula (I), and pharmaceutical compositions comprising a pharmaceutically acceptable carrier material.

Another embodiment of the present invention relates to a compound of formula (Ia) 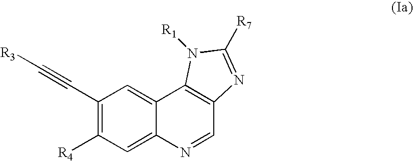
wherein R₁, R₃, R₄ and R₇ are as defined above.

Most preferred is a compound of formula (Ia) wherein
R₁ is substituted or unsubstituted aryl or heteroaryl, especially phenyl which is substituted with up to 4, preferably up to 2 substituents, wherein the substituents are the same or different and are independently selected from halo (e.g. Cl or F); cyano; cyano lower alkyl (e.g. cyanomethyl, cyanoethyl and cyanopropyl); lower alkyl; lower alkoxy; amino-lower alkyl; amino-lower alkoxy; amino-lower alkyl sulfanyl or thiol-lower alkyl; wherein the amino group can be mono or disubstituted, [e.g. —(C₁-C₇)NR₈R₉ or —O—(C₁-C₇)NR₈R₉, wherein R₈ and R₉ can be the same or different and are independently H, lower alkyl (e.g. methyl, ethyl or propyl), lower cycloalkyl (e.g. cyclopropyl) or R₈ and R₉ together with the N atom form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl)]; amino-carbonyl-lower alkyl (e.g. R₈R₉—N—C(O)—CH₂—, wherein R₈ and R₉ are as defined above); heterocyclyl; heterocyclyl-lower alkyl; heterocyclyl-lower alkoxy or heterocyclyl-lower alkanesulfanyl wherein the heterocyclyl is a 3- to 8-membered heterocyclic ring containing 1-4 nitrogen, oxygen or sulfur atoms (e.g. imidazolyl, imidazolinyl, pyrrolidinyl, morpholinyl, azetidinyl, pyridyl, piperidino, piperidyl, piperazinyl or lower alkyl-piperazinyl); wherein alkyl may be linear or cyclic (e.g. cyclopropyl) and the alkyl in any of the substituents above may optionally be substituted with —NR₈R₉, wherein R₈ and R₉ are as defined above;
R₃ is hydrogen; lower alkyl; halo (e.g. fluoro, chloro or bromo); lower alkoxy (e.g. methoxy); unsubstituted or substituted C₅-C₁₄aryl (e.g. phenyl, hydroxyphenyl, methoxyphenyl or aminosulfonyl-phenyl or benzo[1,3]dioxolo); or a heteroaryl being unsubstituted or substituted by one or more, especially 1-4, substituents independently selected from the group consisting of the substituents defined above under “substituted”; especially being pyridyl (or an N-oxide of pyridyl) which is unsubstituted or substituted by one to two radicals selected from the group consisting of lower alkyl (e.g. methyl); lower alkoxy (e.g. methoxy); halo (e.g. fluoro);
or —NR₈R₉, wherein R₈ and R₉ can be the same or different and are independently H, lower alkyl (e.g. methyl, ethyl or propyl); lower cycloalkyl (e.g. cyclopropyl); or the R₈ and R₉ can, with the N atom, form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl);
R₄ is hydrogen or halo, especially fluoro; and
R₇ is hydrogen or an organic moiety, such as C₁-C₇ lower alkyl, amino or amino-lower alkyl; wherein the alkyl may be unsubstituted or substituted with halo (e.g. methyl, ethyl, propyl, trifluoromethyl); lower alkoxy (e.g. methoxy); or cycloalkyl (e.g. cyclopropyl); or a pharmaceutically acceptable salt thereof.

Another embodiment of the present invention relates to a compound of formula (Ib) 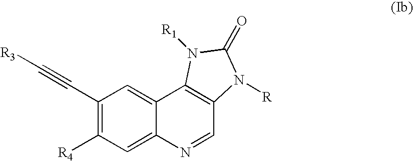
wherein R₁, R₃, R₄, R and y are as defined above.

Most preferred is a compound of formula (Ib),

wherein

R₁ is substituted or unsubstituted aryl or heteroaryl, especially phenyl which is substituted with up to 4, preferably up to 2 substituents, wherein the substituents are the same or different and are independently selected from halo (e.g. Cl or F); cyano; cyano lower alkyl (e.g. cyanomethyl, cyanoethyl and cyanopropyl); lower alkyl; lower alkoxy; amino; amino-lower alkyl; amino-lower alkoxy; amino-lower alkyl sulfanyl or thiol-lower alkyl; wherein the amino group can be mono or disubstituted, [e.g. —(C₁-C₇)NR₈R₉ or —O—(C₁-C₇)NR₈R₉, wherein R₈ and R₉ can be the same or different and are independently H, lower alkyl (e.g. methyl, ethyl or propyl), lower cycloalkyl (e.g. cyclopropyl) or R₈ and R₉ together with the N atom form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl)]; amino-carbonyl-lower alkyl (e.g. R₈R₉—N—C(O)—CH₂—, wherein R₈ and R₉ are as defined above); heterocyclyl; heterocyclyl-lower alkyl; heterocyclyl-lower alkoxy or heterocyclyl-lower alkanesulfanyl wherein the heterocyclyl is a 3- to 8-membered heterocyclic ring containing 1-4 nitrogen, oxygen or sulfur atoms (e.g. imidazolyl, imidazolinyl, pyrrolidinyl, morpholinyl, azetidinyl, pyridyl, piperidino, piperidyl, piperazinyl or lower alkyl-piperazinyl); wherein alkyl may be linear or cyclic (e.g. cyclopropyl) and the alkyl in any of the substituents above may optionally be substituted with —NR₈R₉, wherein R₈ and R₉ are as defined above;
R₃ is hydrogen; lower alkyl; halo (e.g. fluoro, chloro or bromo); lower alkoxy (e.g. methoxy); unsubstituted or substituted C₆-C₁₄aryl (e.g. phenyl, hydroxyphenyl, methoxyphenyl or aminosulfonyl-phenyl or benzo[1,3]dioxolo); or a heteroaryl being unsubstituted or substituted by one or more, especially 1-3, substituents independently selected from the group consisting of the substituents defined above under “substituted”; especially being pyridyl (or an N-oxide of pyridyl) which is unsubstituted or substituted by one to two radicals selected from the group consisting of lower alkyl (e.g. methyl); lower alkoxy (e.g. methoxy); halo (e.g. fluoro);
or —NR₈R₉, wherein R₈ and R₉ can be the same or different and are independently H, lower alkyl (e.g. methyl, ethyl or propyl); lower cycloalkyl (e.g. cyclopropyl); or the R₈ and R₉ can, with the N atom, form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl);
R₄ is hydrogen or halo, especially fluoro; and
R is hydrogen or substituted or unsubstituted C₁-C₇ lower alkyl, aryl, heteroaryl, amino, mono or disubstituted amino, lower alkoxy e.g. OCH₃ or cycloalkyl, e.g. cyclopropyl;
   or a pharmaceutically acceptable salt thereof.

Also preferred is a compound of the formula (Ia) or (Ib), or a pharmaceutically acceptable salt thereof, for use in the preparation of a pharmaceutical composition, or for use in the treatment of a protein kinase dependent disease, especially one depending on PDK1, PI3K and (especially aberrantly highly-expressed or activated) PKB/Akt (=PKB)-dependent disease or a disease dependent on the activation of the PI3K/PKB pathway.

Especially preferred is a compound of the formula (Ia) or (Ib), or a pharmaceutically acceptable salt thereof, wherein X is C═O or CR₇ and the other moieties are as defined under formula (I), for use in the preparation of a pharmaceutical composition, or for use in the diagnostic or therapeutic treatment of a warm-blooded animal, especially a human.

Very preferred is the use of a compound according to formula (I), (Ia) or (Ib), where the disease to be treated is a proliferative disease or conditions which are mediated by the activations of PI3K kinase enzymes, particularly inflammatory or allergic conditions.

Most preferred is the use in accordance with the present invention of a compound of the formula (I), (Ia) or (Ib), or a pharmaceutically acceptable salt thereof, as exemplified below under ‘Examples’.

Especially preferred is a novel compound of formula (I), (Ia) or (Ib), or a pharmaceutically acceptable salt thereof, for use in the therapeutic or diagnostic treatment of a warm-blooded animal, especially a human; or the use of such a novel compound of formula (I), (Ia) or (Ib), or a pharmaceutically acceptable salt thereof, in the treatment of a protein kinase dependent disease or for the manufacture of a pharmaceutical preparation for the treatment of said disease.

Most special preference is further given to the novel compounds of formula (I), (Ia) or (Ib) mentioned in the examples below, or a salt, especially a pharmaceutically acceptable salt, thereof.

PDK1 inhibition can be measured as follows: Cloning and expression: pCMV-GST-PDK1 (G Thomas, FMI Basel, as described in Pullen, N. et al., Science, 279:707-710 (1998)) is digested with EcoR1 and Sma1 to release a DNA fragment encoding amino acids 52-556 of PDK1. This is subsequently ligated to the vector pFB-G01-GST1 with compatible ends achieved by restriction digestion with EcoR1 and Stu1. The ligation reaction is transformed into XL-1 Blue bacteria and plated on selective LB agar. Resultant colonies are cultured overnight, plasmid DNA extracted and restriction analysed. Colonies that are found to contain plasmids with the correct insert are taken for large-scale plasmid preparation and subsequent sequence analysis to confirm the expected plasmid sequence.

Production of virus: Transfer vectors containing the kinase domain of PDK1 are transfected into the DH10Bac cell line (GIBCO) and the cells are plated on selective agar plates. Colonies without insertion of the fusion sequence into the viral genome (carried by the bacteria) are blue. Single, white colonies are picked and viral DNA (bacmid) is isolated from the bacteria by standard plasmid purification procedures. Sf9 cells or Sf21 cells (American Type Culture Collection) are then transfected in 25 cm² flasks with the viral DNA using Celifectin reagent.

Protein expression in Sf9 cells: Virus-containing media is collected from the transfected cell culture and used for infection to increase its titer. Virus-containing media obtained after two rounds of infection is used for large-scale protein expression. For large-scale protein expression 100 cm² round tissue culture plates are seeded with 5×10⁷ cells/plate and infected with 1 mL of virus-containing media (approx. 5 MOIs). After 3 days, the cells are scraped off the plate and centrifuged at 500 rpm for 5 minutes. Cell pellets from 10-20, 100 cm² plates are resuspended in 50 mL of ice-cold lysis buffer (25 mM Tris-HCl, pH 7.5, 2 mM EDTA, 1% NP-40, 1 mM DTT, 1 mMP MSF). The cells are stirred on ice for 15 minutes and then centrifuged at 5,000 rpms for 20 minutes.

Purification of GST-tagged proteins: The centrifuged cell lysate is loaded onto a 2 mL glutathione-sepharose column (Pharmacia) and washed 3× with 10 mL of 25 mM Tris-HCl, pH 7.5, 2 mM EDTA, 1 mM DTT, 200 mM NaCl. The GST-tagged proteins are then eluted by 10 applications (1 mL each) of 25 mM Tris-HCl, pH 7.5, 10 mM reduced-glutathione, 100 mM NaCl, 1 mM DTT, 10% glycerol and stored at −70° C.

Measure of enzyme activity: Tyrosine protein kinase assays with purified GST-PDK1 are carried out in a final volume of 30 μL containing 100 ng of enzyme protein, 50 mM HEPES, pH 7.6, 10 mM MgCl₂, 1 mM DTT, 10 μM Na₃VO₄, 100 μg/mL casein, 1% DMSO, 0.1 mM EGTA, pH 8.0, 10.0 μM ATP and 0.1 μCi [γ-³³P] ATP. The activity is assayed in the presence or absence of inhibitors [compounds of formula (I)] by measuring the incorporation of ³³P from [γ³³P] ATP into appropriate substrates. The assay is carried out in 96-well plates at ambient temperature for 30 minutes under conditions described below and terminated by the addition of 20 μL of 125 mM EDTA. Subsequently, 40 μL of the reaction mixture are transferred onto Immobilon-PVDF membrane (Millipore) previously soaked for 5 minutes with methanol, rinsed with water, then soaked for 5 minutes with 0.5% H₃PO₄ and mounted on vacuum manifold with disconnected vacuum source. After spotting all samples, vacuum is connected and each well-rinsed with 200 μL 0.5% H₃PO₄. Membranes are removed and washed 4× on a shaker with 1.0% H₃PO₄, once with ethanol. Membranes are counted after drying at ambient temperature, mounting in Packard TopCount 96-well frame, and addition of 10 μl/well of Microscint™ (Packard). IC₅₀ values of compounds of formula (I) are calculated by linear regression analysis of the percentage inhibition of each compound in duplicate, at four concentrations (usually 0.01, 0.1, 1 and 10 μM). One unit of protein kinase activity is defined as 1 nmole of ³³P ATP transferred from [γ³³P] ATP to the substrate protein/minute/mg of protein at 37° C.

The compounds of the formula (I) are found to show IC₅₀ values for PDK1 inhibition in the range from 0.001-20 μM, preferably in the range from 0.01-2 μM.

Detection of phospho-PKB and phospho-GSK3β is as follows: On day 1, U87MG cells (ATCC No. HTB-14) are trypsinized, counted in a Neubauer chamber, and diluted in fresh complete RPMI 1640 medium to a final concentration of 6×10⁵ cells/mL. Ten (10) cm tissue culture dishes are then loaded with 10 mL of the cell suspension, and incubated for 18 hours.

On day 2, the medium in plates is discarded and replaced by complete RPMI 1640 medium containing either DMSO or inhibitors [compounds of formula (I)]. After 30 minutes of contact, the medium is quickly removed by aspiration and the cells rinsed twice with pre-cooled PBS. Cells are then placed on ice and immediately lysed. Protein samples are then resolved by SDS-PAGE and transferred to Immbilon-P membrane for detection of levels of endogenous GSK3β, PKB, PhosphoT308-PKB and PhosphoS9-GSK3β by western-blotting. Membranes are then dried and covered with polyethylene film, and chemiluminescence measured in a MultiImage™ Light Cabinet (Alpha Innotech Corp) driven with the Fluor Chem™ software (Alpha Innotech Corp).

The data are analyzed with AphaEasy software, plotted as % of control (cells treated with DMSO in identical experimental conditions used for kinase inhibitors) with SigmaPlot® (SSPI Inc, version 7) as a regression curve (Four Parameter Logistic Cubic) and IC₅₀ values are determined accordingly.

IC₅₀ calculations

| | |
|---|---|
| input | 3 × 4 μl stopped assay on Immobilon membrane, |
| | not washed |
| background (3 wells) | assay with H₂O instead of enzyme. |
| positive control (4 | 3% DMSO instead of compound |
| wells) | |
| bath control (1 well) | no reaction mix |

IC₅₀ values are calculated by logarithmic regression analysis of the percentage inhibition of each compound at 4 concentrations (usually 3- or 10-fold dilution series starting at 10 μM).

In each experiment, the actual inhibition by reference compound is used for normalization of IC₅₀ values to the basis of an average value of the reference inhibitor:
Normalized IC₅₀=sured IC₅₀·average ref. IC₅₀/measured ref. IC₅₀
Example: Reference inhibitor in experiment 0.4 μM, average 0.3 μM
Test compound in experiment 1.0 μM, normalization: 0.3/0.4=0.75 μM

For example, staurosporine or a synthetic staurosporine derivative are used as reference compounds.

Using this protocol, the compounds of the formula (I) are found to show IC₅₀ values for PDK1 Inhibition in the range from 0.001-20 μM, preferably in the range from 0.01-2 μM.

Compounds of formula I and their pharmaceutically acceptable salts are useful as pharmaceuticals. In particular, they exhibit Inhibition of phosphatidylinositol 3-kinase (PI3K kinase) enzymes, especially the gamma isoform (p110y), which are responsible for generating phosphorylated signalling products. The inhibitory properties of compounds of formula I may be demonstrated in the following test procedures:

Baculovirus expressing different fragments of PI3Ky fused to GST have been previously described by Stoyanova et al. (1997) Lipid- and protein kinase activities of G protein-coupled PI 3-kinase g: structure-activity analysis and interactions with wortmannin. Biochem. J., 324:489. Residues 38-1102 of human PI3Ky are subcloned into the BamH1 and EcoR1 sites of the transfer vector pAcG2T (Pharmingen) to create a GST-PI3Ky lacking the first 37 residues of PI13Ky. To express the recombinant protein, Sf9 (Spodoptera frugiperda 9) insect cells are routinely maintained at densities between 3×10⁵ and 3×10⁶ cells/ml in serum containing TNMFH medium (Sigma). Sf9 cells, at a density of 2×10⁶ are infected with human GST-PI3KyΔ34 baculovirus at a multiplicity of infection (m.o.i.) of 1 for 72 hours. The infected cells are harvested by centrifugation at 1400 g for 4 minutes at 4° C. and the cell pellets are frozen at −80° C. Both Sf9 and Sf21 cells work equally well. Sf9 cells (1×10⁹) are resuspended in 100 ml cold (4° C.) lysis buffer (50 mM Tris-HCl pH 7.5, 1% Triton X-100, 150 mM NaCl, 1 mM NaF, 2 mM DTT and protease inhibitors. Cells are incubated on ice for 30 minutes then centrifuged at 15000 g for 20 minutes at 4° C. Purification of the supernatant sample is carried out at 4° C. by affinity chromatography using SEPHAROSE™ agarose gel beads coupled to glutathione (from Amersham Pharmacia Biotech). A cell lysate/GST resin ratio of 50:1 is used. The GST resin is firstly pre-rinsed to remove ethanol preservative and then equilibrated with lysis buffer. Cell lysate (supernatant) is added (usually as 50 ml lysate to 1 ml GST resin in 50 ml tubes) and gently rotated on a mixer at 4° C. for 2-3 hours. The unbound flow through sample is collected by centrifugation at 1000 g for 5 minutes at 4° C. using a DENLEY™ centrifuge. The 1 ml GST resin containing bound material is transferred to a 15 ml FALCON™ centrifuge tube for subsequent washing and elution steps. Firstly a series of 3 cycles of washings (mixing by gentle inversion) is performed with 15 ml ice cold wash Buffer A (50 mM Tris-HCl pH 7.5, 1% Triton X-100, 2 mM DTT) interspersed with centrifugation at 1000 g for 5 minutes at 4° C. A final single wash step is performed with 15 ml ice cold wash Buffer B (50 mM Tris-HCl pH 7.5, 2 mM DTT) and then centrifuged at 100 g for 5 minutes at 4° C. The washed GST resin is finally eluted with 4 cycles of 1 ml ice cold elution buffer (50 mM Tris-HCl pH 7.5, 10 mM reduced glutathione, 2 mM DTT, 150 mM NaCl, 1 mM NaF, 50% ethylene glycol and protease inhibitors) interspersed with centrifugation at 1000 g for 5 minutes at 4° C. Samples are aliquoted and stored at −20° C.

An in vitro kinase assay was established that measures the transfer of the terminal phosphate of adenosine triphosphate to phosphatidylinositol. The kinase reaction is performed in a white 96 well microtitre plate as a Scintillation Proximity Assay. Each well contains 10 μl test compound in 5% dimethylsulphoxide and 20 μl assay mix (40 mM Tris, 200 mM NaCl, 2 mM ethyleneglycol-aminoethyl-tetraacetic acid (EGTA), 15 μg/ml phosphatidylinositol, 12.5 μM adenosine triphosphate (ATP), 25 mM MgCl₂, 0.1 μCi [³³P]ATP). The reaction is started by the addition of 20 μl of enzyme mix (40 mM Tris, 200 mM NaCl, 2 mM EGTA containing recombinant GST-p110y). The plate is incubated at room temperature for 60 minutes and the reaction terminated by the adding 150 μl of WGA-bead stop solution (40 mM Tris, 200 mM NaCl, 2 mM EGTA, 1.3 mM ethylene diamine tetraacetic acid (EDTA), 2.6 μM ATP and 0.5 mg of Wheat Germ Agglutinin-SPA beads (Amersham Biosciences) to each well. The plate is sealed, incubated at room temperature for 60 minutes, centrifuged at 1200 rpm and then counted for 1 minute using a scintillation counter. Total activity is determined by adding 10 μl of 5% dimethylsulphoxide (DMSO) and non-specific activity is determined by adding 10 μl 50 mM EDTA in place of the test compound.

The compounds of formula (I) that inhibit the protein kinase activities mentioned, especially tyrosine and/or the serine/threonine protein kinases mentioned above, can therefore be used in the treatment of protein kinase dependent diseases, especially diseases depending on PDK1 kinases activity. Protein kinase dependent diseases are especially proliferative diseases, preferably a benign or especially malignant tumor, more preferably carcinoma of the brain, kidney, liver, adrenal gland, bladder, breast, stomach (especially gastric tumors), ovaries, colon, rectum, prostate, pancreas, lung, vagina, thyroid, sarcoma, glioblastomas, multiple myeloma or gastrointestinal cancer, especially colon carcinoma or colorectal adenoma, or a tumor of the neck and head, an epidermal hyperproliferation, especially psoriasis, prostate hyperplasia, a neoplasia, especially of epithelial character, preferably mammary carcinoma, or a leukemia. They are able to bring about the regression of tumors and to prevent the formation of tumor metastases and the growth of (also micro) metastases. In addition they can be used in epidermal hyperproliferation (e.g. psoriasis), in prostate hyperplasia, in the treatment of neoplasias, especially of epithelial character, for example, mammary carcinoma, and in leukemias. It is also possible to use the compounds of formula (I) in the treatment of diseases of the Immune system insofar as several or, especially, individual tyrosine protein kinases and/or (further) serine/threonine protein kinases are involved; furthermore, the compounds of formula (I) can be used also in the treatment of diseases of the central or peripheral nervous system where signal transmission by at least one tyrosine protein kinase and/or (further) serine/threonine protein kinase is involved.

Especially compounds of formula (I) that show inhibition of PDK1 kinase are useful in the treatment of PTEN negative cancers or cancers that overexpress PKB or PI3K or diseases associated with deregulation of the PI3K/PKB pathway.

Further, compounds of the invention are useful in the treatment of inflammatory or obstructive airways diseases, resulting, for example, in reduction of tissue damage, airways inflammation, bronchial hyper-reactivity, remodelling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as Incipient or early-phase asthmatics. (This particular asthmatic condition is commonly referred to as “wheezy-infant syndrome”.)

Prophylacetic efficacy in the treatment of asthma will be evidenced by reduced frequency or severity of symptomatic attack, e.g. of acute asthmatic or bronchoconstrictor attack, improvement in lung function or improved airways hyper-reactivity. It may further be evidenced by reduced requirement for other, symptomatic therapy, i.e. therapy for or intended to restrict or abort symptomatic attack when it occurs, for example anti-inflammatory (e.g. corticosteroid) or bronchodilatory. Prophylacetic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant form any previously administered symptomatic asthma therapy.

Other inflammatory or obstructive airways diseases and conditions to which the present invention is applicable include acute lung injury (ALI), adult respiratory distress syndrome (ARDS), chronic obstructive pulmonary, airways or lung disease (COPD, COAD or COLD), including chronic bronchitis or dyspnea associated therewith, emphysema, as well as exacerbation of airways hyper-reactivity consequent to other drug therapy, in particular other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an Inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, cystic fibrosis, ptilosis, siderosis, silicosis, tabacosis and byssinosis.

Having regard to their anti-inflammatory activity, in particular in relation to inhibition of eosinophil activation, compounds of the invention are also useful in the treatment of eosinophil related disorders, e.g. eosinophilia, in particular eosinophil related disorders of the airways (e.g. Involving morbid eosinophilic infiltration of pulmonary tissues) including hyper-eosinophilia as it effects the airways and/or lungs as well as, for example, eosinophil-related disorders of the airways consequential or concomitant to Löffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Compounds of the invention are also useful in the treatment of inflammatory or allergic conditions of the skin, for example psoriasis, contact dermatits, atopic dermatitis, alopecia greata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, and other inflammatory or allergic conditions of the skin.

Compounds of the present invention may also be used for the treatment of other diseases or conditions, in particular diseases or conditions having an inflammatory component, for example, treatment of diseases and conditions of the eye such as conjunctivitis, keratoconjunctivitis sicca, and vernal conjunctivitis, diseases affecting the nose including allergic rhinitis, and inflammatory disease in which autoimmune reactions are implicated or having an autoimmune component or aetiology, including autoimmune haematological disorders (e.g. haemolytic anaemia, aplastic anaemia, pure red cell anaemia and idiopathic thrombocytopenia), systemic lupus erythematosus, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary billiary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. Including idiopathic nephrotic syndrome or minal change nephropathy).

Other diseases or conditions which may be treated with compounds of the invention include septic shock, rheumatoid arthritis, osteoarthritis, proliferative diseases such as cancer, atherosclerosis, allograft rejection following transplantation, stroke, obesity, restenosis, diabetes, e.g. diabetes mellitus type I auvenile diabetes) and diabetes mellitus type II, diarrhoeal diseases, ischemia/reperfusion injuries, retinopathy, such as diabetic retinopathy or hyperbaric oxygen-induced retinopathy, and conditions characterised by elevated intraocular pressure or secretion of ocular aqueous humor, such as glaucoma.

The effectiveness of compounds of the invention in inhibiting inflammatory conditions, for example in inflammatory airways diseases, may be demonstrated in an animal model, e.g. a mouse or rat model, of airways inflammation or other inflammatory conditions, for example as described by Szarka et al., J. Immunol. Methods (1997) 202:49-57; Renzi et al., Am. Rev. Respir. Dis. (1993) 148:932-939; Tsuyuki et al., J. Clin. Invest. (1995) 96:2924-2931; and Cernadas et al. (1999) Am. J. Respir. Cel Mol. Biol. 20:1-8.

There are also experiments to demonstrate the antitumor activity of compounds of the formula (I) in vivo.

Female Harlan athymic nu/nu mice with s.c. transplanted human glioblastoms U87MG tumors can be used to determine toe anti-tumor activity of PDK1 kinase inhibitors. On day 0, with the animals under peroral forene narcosis, a tumor fragment of approximately 25 mg is placed under the skin on the animals' left flank and the small incised wound is closed by means of suture clips. When tumors reaches a volume of 100 mm³ the mice are divided at random into groups of 6-8 animals and treatment commences. The treatment is carried out for a 2-3 weeks period with peroral, intravenous or intra-peritoneal administration once daily (or less frequently) of a compound of formula (I) in a suitable vehicle at defined doses. The tumors are measured twice a week with a slide gauge and the volume of the tumors is calculated.

As an alternative to cell line U87MG, other cell lines may also be used in the same manner, for example,
the MDA-MB 468 breast adenocarcinoma cell line (ATCC No. HTB 132; see also In Vitro 14, 911-15 [1978]);
the MDA-MB 231 breast carcinoma cell line (ATCC No. HTB-26; see also In Vitro 12, 331 [1976]);
the MDA-MB 453 breast carcinoma cell line (ATCC No. HTB-131);
the Colo 205 colon carcinoma cell line (ATCC No. CCL 222; see also Cancer Res. 38, 1345-55 [1978]);
the DU145 prostate carcinoma cell line DU 145 (ATCC No. HTB 81; see also Cancer Res. 37, 4049-58 [1978]),
the PC-3 prostate carcinoma cell line PC-3 (especially preferred; ATCC No. CRL 1435; see also Cancer Res. 40, 524-34 [1980]) and the PC-3M prostate carcinoma cell line;
the A549 human lung adenocarcinoma (ATCC No. CCL 185; see also Int. J. Cancer 17, 62-70 [1976]),
the NCI-H596 cell line (ATCC No. HTB 178; see also Science 246, 491-4 [1989]);
the pancreatic cancer cell line SUIT-2 (see Tomioka et al., Cancer Res. 61, 7518-24 [2001]).

Other cell lines include gioblastoma cell lines that are PTEN negative (see Ishii et al., Brain Pathology 9, 469-479 [1999]), such as
LN-71;
LN-215;
LN-235.

The compounds of the formula (I) can be prepared according to the following methods:

In one preferred embodiment, a compound of formula (I) is prepared by reacting a compound of the formula (II) 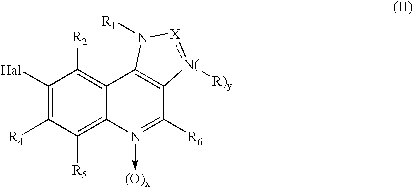
with an alkenylene or alkynylene derivative, preferably phenylethylene boronic acid, phenylacetylene, 3-methoxyphenylacetylene, 4-methoxyphenylacetylene, 3-ethynylpyridine, 5-ethynyl-2-methoxy-pyridine, 5-ethynyl-benzo[1,3]dioxolo or 4-ethynyl-benzenesulfonamide, wherein
Hal refers to halogen preferably bromine; and
x, y, X, R₁, R₂, R₄, R₅ and R₆ are as defined above; and
   If desired, transforming an obtainable compound of formula (I) into a different compound of formula (I), transforming a salt of an obtainable compound of formula (I) into the free compound or a different salt, or an obtainable free compound of formula (I) into a salt; and/or separating an obtainable mixture of isomers of compounds of formula (I) into the individual isomers.

In the following, more detailed description of the preferred process conditions, x, y, R₁, R₂, R₄, R₃, R₅, R₆, R₇, X, and R have the meanings given for compounds of the formula (I), if not indicated otherwise.

Starting Materials

A compound of formula (II) of the first preferred embodiment is prepared by reacting a compound of formula (IIa) 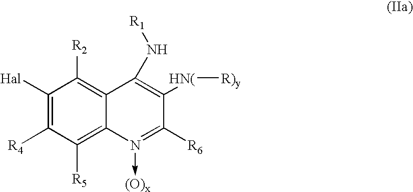
wherein
x, y, R₁, R₂, R₄, R₅ and R₆ are as mentioned for a compound of the formula (I); and
R is as defined below under a), b) or c), respectively,
a) for the manufacture of a compound of the formula (II), wherein X is C═O and the dashed line in formula (I) bonding X to N is absent, y is 1 and R is hydrogen or an organic moiety that can be bound to nitrogen, with an active derivative of a compound of the formula (III)
   A-X-A (III)
   wherein X is C═O and each A, independently of the other, is a carbonyl-activating group;
b) for the manufacture of a compound of the formula (II), wherein X is C═S and the dashed line in formula (I) bonding X to N is absent, y is 1 and R is hydrogen or an organic moiety that can be bound to nitrogen, with CS₂ or Cl—C(═S)—Cl; or
c) for the manufacture of a compound of the formula (II), wherein X is (CR₇) wherein R₇ is hydrogen or an organic or inorganic moiety with the proviso that then the dashed line bonding X to N is a bond, so that X is bound to the adjacent N via a double bond, with an activated derivative of a compound of formula (IVa), (IVb) or (IVc) or a derivative of one of these compounds:
   R₇—COOH (IVa)
   R₇—CN (IVb)
   R₇—CHO (IVc)
   wherein R₇ is hydrogen, an organic or inorganic moiety, especially C₁-C₇lower alkyl, amino or amino lower alkyl;
wherein functional groups which are present in the starting compounds in processes a) to c) and are not intended to take part in the reaction, are present in protected form if necessary, and protecting groups that are present are cleaved, wherein said starting compounds may also exist in the form of salts provided that a salt-forming group is present and a reaction in salt form is possible.

A compound of the formula (II), wherein R is hydrogen and y is 1 is preferably prepared by hydrogenation of a compound of the formula (V) 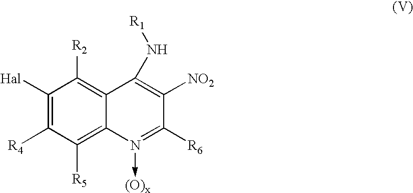
wherein the substituents and symbols are defined as for compounds of the formula (I) (x is preferably zero), in the presence of an appropriate catalyst, e.g. a skeleton based catalyst, such as Raney-Ni, with hydrogen in an appropriate solvent, e.g. an alcohol, such as methanol, at preferred temperatures between 0° C. and 50° C., e.g. at room temperature.

The corresponding compounds of the formula (II), wherein R is an organic moiety that can be bound to nitrogen, especially a carbon-bound one, can be prepared by reaction of a compound of formula (II), wherein R is hydrogen and y is 1 (see preceding paragraph) with a compound of the formula (VI)
R-L (VI)
wherein R is an organic moiety bound to L via a carbon atom and L is a leaving group, especially halo, such as chloro, bromo or iodo, or arylsulfonyl, e.g. toluenesulfonyl, in an appropriate solvent, preferably in the presence of a tertiary nitrogen base, such as pyridine or triethylamine.

Alternatively, a compound of the formula (II), wherein R is hydrogen and y is 1 can be reacted with a carbonyl containing compound of the formula (VI*) or (VI**)
R*—CHO (VI*)
R*—CO—R** (VI**)
wherein R* and R** are the same or different and each is as an organic moiety bound to the CO moiety via a carbon atom, followed by reduction of the resulting enamine with an appropriate reductant, e.g. a complex hydride, such as an alkalimetal cyanoborohydride, e.g. sodium-cyanoborohydride, e.g. in the same solvent and at temperatures between −10° C. and 40° C., e.g. at 10° C., the total reaction summing up to reductive amination.

A compound of formula (V) is preferably prepared by reacting a compound of the formula (VII) 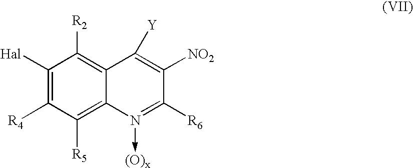
wherein Y is halo, especially chloro, and the other moieties and symbols have the meanings indicated for compounds of the formula (I) (x is preferably zero), with a compound of the formula (VIII)
R₁—NH₂ (VIII)
wherein R₁ is as defined for a compound of the formula (I), in an appropriate solvent, preferably a lower alkylcarboxylic acid, such as acetic acid, at preferred temperatures between 10 C and reflux temperature of the reaction mixture, e.g. between 20° C. and 140° C.

A compound of the formula (VII) can be prepared by reacting a compound of the formula (IX) 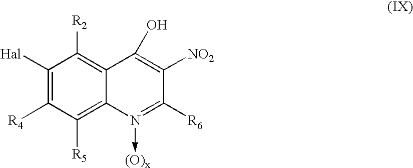
wherein the moieties and symbols have the meanings indicated for a compound of the formula (I) (x is preferably zero), with an inorganic acid halogenide, especially POCl₃ (preferably without solvent) at elevated temperatures, e.g. between 100° C. and 150° C. or under reflux.

A compound of the formula (IX) is known in the art, can be synthesized according to methods known in the art and/or is commercially-available. For example, it can be synthesized by reacting a compound of the formula (X) 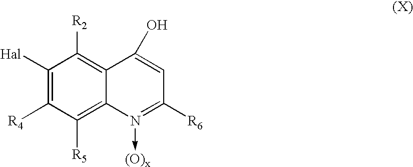
wherein the moieties and symbols have the meanings indicated for a compound of the formula (I) (x is preferably zero) with nitric acid (aqueous) at a preferred temperature between 50° C. and 100° C., e.g. at 85° C.

A compound of the formula (IX), can alternatively be synthesized by reacting a compound of the formula (XI) 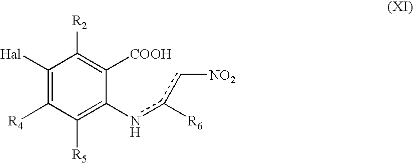
wherein the moieties and symbols have the meanings indicated for a compound of the formula (I), with an anhydride of a carbonic acid, especially acetic anhydride, preferably in the presence of an alkali metal salt of a carboxylic acid, e.g. potassium acetate, at a preferred temperature between 50° C. and 150° C., e.g. at ca. 100-140° C.

A compound of the formula (XI) can be obtained, for example, by converting a compound of the formula (XII) 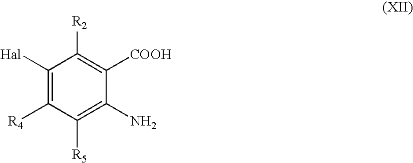
to the corresponding compound of the formula (XI) by reacting nitromethane in the presence of an alkali metal hydroxide, especially sodium hydroxide, at preferred temperatures between approximately 0° C. and 60° C., e.g. between 0° C. and room temperature, then pouring the product under cooling to approximately 0° C. into conc. HCl and adding the compound of the formula (XII) and further conc. HCl, subsequently allowing for further reaction at preferred temperatures between 0° C. and room temperature to result in the corresponding compound of formula (XI).

Other starting materials are either known in the art, can be prepared according to methods that are known in the art, e.g. in analogy to the methods described hereinabove or in the examples, and/or are commercially-available.

The present invention relates also to novel starting materials and/or intermediates and to processes for their preparation. The starting materials used and the reaction conditions selected are preferably those that result in the compounds described as being preferred.

Detailed Description of Preferred Reaction Conditions

The reaction described under (a) preferably takes place under conditions known in the art, especially in an appropriate solvent, such as a halo-lower alkane, e.g. dichloromethane, or a lower alkylnitrile, such as acetonitrile, and under elevated temperatures, preferably in the range from 40° C. to the reflux temperature of the reaction mixture, especially under reflux. In the compound of the formula (III), each A is, independently of the other, preferably halo, trichloromethyl, succinimido or 1-imidazolo. For example, if the compound of the formula (III) is trichloromethyl chloroformate, the reaction preferably takes place under anhydrous conditions in an appropriate aprotic solvent, e.g. a halogenated hydrocarbon, such as dichloromethane, at preferred temperatures between 0° C. and 50° C., e.g. at room temperature.

The reaction described under (b) with CS₂ or Cl—C(═S)—Cl preferably takes place in the presence of a base, especially a tertiary amine, such as tri-lower alkylamine, preferably triethylamine, or pyridine, an alkalimetal carbonate or -bicarbonate, e.g. sodium bicarbonate, or a metal hydroxide, especially an alkali metal hydroxide, such as sodium- or potassium hydroxide, in a polar organic solvent, especially an alcohol, at temperatures between 10° C. and the reflux temperature, more preferably between 20° C. and 100° C.

The reaction described under (c) preferably takes place in the presence of an active derivative of a compound of the formula (IVa), (IVb) and (IVc) as solvent or other appropriate solvents or solvent mixtures at preferred temperatures between 30° C. and the reflux temperature of the reaction mixture, more preferably under reflux. An activated derivative of a compound of the formula (IVa) is especially a tri-lower alkyl orthoester of the carbonic acid of formula (IVa), especially a tri-ethyl derivative, such as triethylorthoformate or a tetramethyl derivative, such as tetramethyl orthocarbonate. Alternatively, the respective reactive derivative of an acid of the formula (IVa) is formed in situ, e.g. in the presence of polyphosphoric acid (also as solvent) at elevated temperatures, e.g. between 100° C. and 140° C. An activated derivative of a compound of formula (IVb) is especially a halo derivative, such as cyanogen bromide.

Compounds of formula (I) can be transformed into different compounds of formula (I).

Especially, the following transformations are of interest

In compounds of the formula (I), wherein R₁ carries a cyano or cyano-lower alkyl substituent, this substituent can be converted into an aminomethyl or aminomethyl-lower alkyl group, respectively, by hydrogenation, e.g. with hydrogen in the presence of an appropriate catalyst, such as a Raney catalyst, especially Raney-Ni, in an appropriate solvent, such as an alcohol, especially methanol or ethanol, or a cyclic ether, such as tetrahydrofuran, or a mixture thereof, in the presence of ammonia, preferably at temperatures between 0° C. and 50° C., e.g. at room temperature.

In compounds of the formula (I), wherein R₁ carries a cyano or cyano-lower alkyl substituent or R₇ is any one of these substituents, this substituent can be converted into a N-hydroxyamidino or N-hydroxyamidino-lower alkyl group, respectively, by reaction with a hydroxylamine salt of an organic or inorganic acid, e.g. a hydroxylamine halogenide, in a polar solvent, e.g. a di-lower alkyl lower alkanoylamide, especially dimethyl formamide, in the presence of water at preferred temperatures between 10° C. and 100° C., e.g. at 20-75° C., in the presence of a base, especially an alkali metal carbonate, such as sodium carbonate.

In compounds of the formula (I), wherein R₁ is 2-haloaryl, e.g. 2-chlorophenyl, the halogen can be removed by hydrogenation with hydrogen in an appropriate solvent, e.g. in an alcohol, such as methanol, or a N,N-di-lower alkyl-loweralkanoylamide, such as dimethylformamide, or a mixture thereof, and a catalyst, such as a noble metal on a carrier material, e.g. palladium on charcoal (Pd—C), at preferred temperatures between 0° C. and 50° C., e.g. at room temperature, to the corresponding compound wherein R₁ is aryl, e.g. phenyl.

In a compound of the formula (I), wherein a hydroxyamidino substituent is present (e.g. as mentioned in the last paragraph), this substituent can be converted into the corresponding amidino substituent by hydrogenation in the presence of an acid, such as hydrochloric acid, and a catalyst, preferably a Raney metal catalyst, such as Raney-Ni, preferably at elevated temperatures, e.g. between 30° C. and 70° C., e.g. at 50° C.

Compounds of the formula (I), wherein x and y or one of them are zero can be converted into the corresponding N-oxide compounds (x, y or both=1, R=→O) by oxidation in the presence of a peroxide, especially a peroxybenzoic acid derivative, such as 3-chloroperoxybenzoic acid, in the presence of a base, e.g. an alkali metal carbonate, such as sodium carbonate, and in an appropriate solvent, e.g. a halogenated hydrocarbon, such as chloroform or dichloromethane.

Compound of formula (I), where X is CR₇ and R₇ is NH₂ is prepared from the corresponding di-amino compound and cyanogen bromide in an appropriate solvent, e.g. ethanol, at temperatures between 0° C. and 50° C., e.g. room temperature.

A compound of formula (I), where X is CR₇ and R₇ is OCH₃ is prepared from the corresponding di-amino compound and tetramethyl orthocarbonate in the presence of an appropriate solvent, e.g. acetic acid, at elevated temperatures, e.g. 75° C.

A compound of formula (I), where X is CR₇ and R₇ is CF₃ is prepared from the di-amino compound and trifluoroacetic acid in the presence of an appropriate solvent, e.g. 4 N HCl, at elevated temperatures, e.g. 100° C.

A compound of formula (I) where X is CR₇ and R₇ is CH₃ is prepared from the corresponding di-amino compound and triethylorthoacetate at elevated temperatures, e.g. 130° C.

A compound of formula (I), where X is CR₇ and R₇ is lower alkyl is prepared from the corresponding di-amino compound and the corresponding aldehyde using catalytic amounts of acetic acid in an appropriate solvent, e.g. DCM, at temperatures between 0° C. and 50° C., e.g. room temperature.

A compound of formula (I), where G is an alkenylene is prepared from the corresponding halo-derivative by reaction with a boronic acid, e.g. trans-phenylethenyl-boronic acid, in the presence of a catalyst, e.g. bis(triphenylphosphine)palladium(II) dichloride in potassium carbonate in DMF at elevated temperatures, 100° C., and under an inert atmosphere, e.g. an argon atmosphere.

A compound of formula (I), where G is and alkynylene is prepared by Sonogashira coupling. See Sonogashira et al., Tetrahedron Lett, p. 44671 (1975). The corresponding halo-derivative is reacted with the corresponding acetylene, e.g. phenylacetylene, in the presence of CuI, bis(benzonitrile)palladium (II) dichloride, tri-tert-butylphosphine, and diisopropylamine in dioxane, in an inert atmosphere, e.g. argon atmosphere.

A compound of the formula (I), wherein x is 1 and R₆ is hydrogen can be transformed into the corresponding compound wherein x is zero an R₆ is halo by reaction with an inorganic halogenide, e.g. POCl₃, in an appropriate solvent, e.g. a mixture of a di-lower alkyl alkanoylamide, such as dimethylformamide, and an aromatic hydrocarbon, e.g. toluene, at elevated temperatures, e.g. between 50° C. and 90° C.

A compound of the formula (I), wherein R₆ is halo can be converted into a compound of the formula (I), wherein R₆ is amino substituted by one or two moieties selected from the group consisting of lower alkyl, substituted lower alkyl moieties, aryl, cycloalkyl and mercapto-lower alkyl by reaction with the corresponding primary or secondary amine, respectively, in an appropriate solvent, e.g. an alcohol, especially methanol or 2-ethoxyethanol, at temperatures between 100° C. and 130° C. (if necessary in a sealed reaction vessel, e.g. a sealed tube).

A compound of the formula (I), wherein X is (CR₇) and R₇ is halogen can be obtained from the corresponding compound wherein R₇ is hydrogen by reaction with the corresponding halogen succinimide, especially N-bromosuccinimide, in the presence of the corresponding iron(II)halogenide, especially FeBr₃, in the absence or presence of an appropriate solvent at elevated temperatures, preferably under reflux.

A compound of the formula (I), wherein X is (CR₇) and R₇ is cyano can be obtained from the corresponding compound wherein R₇ is —CONH₂ by reaction with an inorganic acid halogenide, especially POCl₃, in an appropriate base, especially pyridine, preferably at elevated temperatures, more preferably between 25° C. and 80° C. Alternatively, the compound can be obtained from a compound of the formula (I), wherein R₇ is bromo (as obtainable in the last paragraph) by reaction in the presence of CuCN and a catalyst, especially), tris(dibenzylideneacetone)dipalladium chloroform adduct and 1,1′-bis(diphenylphosphino)ferrocene, and of tetraethylammonium cyamide in an appropriate solvent, e.g. a cyclic ether, such as dioxane, at preferred temperatures (if necessary in a sealed tube) between 100° C. and 150° C., e.g. at 140° C.

A compound of the formula (I), wherein X is C═O, y is 1 and R is unsubstituted or substituted alkyl, especially lower alkyl, can be obtained by converting the corresponding compound of the formula (I), wherein R is H with a halogenide, especially iodide, such as lower alkyl iodide, in the presence of a strong base, especially an alkali metal hydride, e.g. sodium hydride, in an appropriate aprotic solvent, e.g. a N,N-di-lower alkyl-lower alkanoylamide, at preferred temperatures in the range from 0-50° C., e.g. at room temperature, into said compound.

A compound of the formula (I), wherein X is C═O, y is 1 and R is aryl, especially phenyl, can be obtained by converting the corresponding compound of the formula (I), wherein R is H with an arylboronic acid, especially phenylboronic acid, in the presence of anhydrous cupric acetate and a tertiary amine, e.g. a tri-lower alkylamine, such as triethylamine, in an appropriate aprotic solvent, especially a halogenated hydrocarbon, such as dichloromethylene, at preferred temperatures between 0° C. and 50° C., e.g. at room temperature, into said compound.

Salts of compounds of formula (I) having at least one salt-forming group may be prepared in a manner known per se. For example, salts of compounds of formula (I) having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of formula (I) are obtained in customary manner, e.g. by treating the compounds with an acid or a suitable anion exchange reagent. Internal salts of compounds of formula (I) containing acid and basic salt-forming groups, e.g. a free carboxy group and a free amino group, may be formed, e.g. by the neutralization of salts, such as acid addition salts, to the isoelectric point, e.g. with weak bases, or by treatment with Ion exchangers.

Salts can be converted in customary manner into the free compounds; metal and ammonium salts can be converted, for example, by treatment with suitable acids, and acid addition salts, for example, by treatment with a suitable basic agent.

Mixtures of isomers obtainable according to the invention can be separated in a manner known per se into the individual isomers; diastereoisomers can be separated, for example, by partitioning between polyphasic solvent mixtures, recrystallization and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallization, or by chromatography over optically active column materials.

Intermediates and final products can be worked up and/or purified according to standard methods, e.g. using chromatographic methods, distribution methods, (re-)crystallization and the like.

Additional Process Steps

In the additional process steps, carried out as desired, functional groups of the starting compounds which should not take part in the reaction may be present in unprotected form or may be protected for example by one or more protecting groups. The protecting groups are then wholly or partly removed according to one of the known methods.

Protecting groups, and the manner in which they are introduced and removed are described, for example, in “Protective Groups in Organic Chemistry”, Plenum Press, London, New York 1973, and in “Methoden der organischen Chemie”, Houben-Weyl, 4th edition, Vol. 15/1, Georg-Thieme-Verlag, Stuttgart 1974 and in Theodora W. Greene, “Protective Groups in Organic Synthesis”, John Wiley & Sons, New York 1981. A characteristic of protecting groups is that they can be removed readily, i.e. without the occurrence of undesired secondary reactions, for example, by solvolysis, reduction, photolysis, acidolysis or alternatively under physiological conditions.

The end products of formula (I) may however also contain substituents that can also be used as protecting groups in starting materials for the preparation of other end products of formula (I). Thus, within the scope of this text, only a readily removable group that is not a constituent of the particular desired end product of formula (I) is designated a “protecting group”, unless the context indicates otherwise.

General Process Conditions

The following applies in general to all processes mentioned hereinbefore and hereinafter, while reaction conditions specifically mentioned above or below are preferred:

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, for example, ion exchangers, such as cation exchangers, e.g. in the H⁺ form, depending on the nature of the reaction and/or of the reactants at reduced, normal or elevated temperature, for example, in a temperature range of from about −100° C. to about 190° C., preferably from approximately −80° C. to approximately 150° C., for example, at from −80 to −60° C., at room temperature, at from −20 to 40° C. or at reflux temperature, under atmospheric pressure or in a closed vessel, where appropriate under pressure, and/or in an inert atmosphere, for example, under an argon or nitrogen atmosphere.

At all stages of the reactions, mixtures of isomers that are formed can be separated into the individual isomers, for example, diastereoisomers or enantiomers, or into any desired mixtures of isomers, for example, racemates or mixtures of diastereoisomers, for example, analogously to the methods described under “additional process steps”.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example, diethyl ether, or cyclic ethers, for example, tetrahydrofuran or dioxane, liquid aromatic hydrocarbons, such as benzene or toluene, alcohols, such as methanol, ethanol or 1- or 2-propanol, nitrites, such as acetonitrile, halogenated hydrocarbons, such as dichloromethane or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in new compounds of formula (I) described at the beginning as being especially valuable. Special preference is given to reaction conditions that are analogous to those mentioned in the examples.

Pharmaceutical Compositions

The invention relates also to pharmaceutical compositions comprising a compound of formula (I), to their use in the therapeutic (in a broader aspect of the invention also prophylacetic) treatment or a method of treatment of a protein kinase dependent disease, especially the preferred diseases mentioned above, to the compounds for said use and to the preparation of pharmaceutical preparations, especially for said uses.

The present invention also relates to pro-drugs of a compound of formula (I) that convert in vivo to the compound of formula (I) as such. Any reference to a compound of formula (I) is therefore to be understood as referring also to the corresponding pro-drugs of the compound of formula (I), as appropriate and expedient.

The pharmacologically acceptable compounds of the present invention may be used, for example, for the preparation of pharmaceutical compositions that comprise an effective amount of a compound of the formula (I), or a pharmaceutically acceptable salt thereof, as active ingredient together or in admixture with a significant amount of one or more inorganic or organic, solid or liquid, pharmaceutically acceptable carriers.

The invention relates also to a pharmaceutical composition that is suitable for administration to a warm-blooded animal, especially a human (or to cells or cell lines derived from a warm-blooded animal, especially a human, e.g. lymphocytes), for the treatment or, in a broader aspect of the invention, prevention of (=prophylaxis against) a disease that responds to inhibition of protein kinase activity, comprising an amount of a compound of formula (I) or a pharmaceutically acceptable salt thereof, which is effective for said inhibition, especially the in, together with at least one pharmaceutically acceptable carrier.

The pharmaceutical compositions according to the invention are those for enteral, such as nasal, rectal or oral, or parenteral, such as intramuscular or intravenous, administration to warm-blooded animals (especially a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The invention relates also to a method of treatment for a disease that responds to inhibition of a protein kinase; which comprises administering an (against the mentioned disease) prophylactically or especially therapeutically effective amount of a compound of formula (I) according to the invention, especially to a warm-blooded animal, for example a human, that, on account of one of the mentioned diseases, requires such treatment.

The dose of a compound of the formula (I) or a pharmaceutically acceptable salt thereof to be administered to warm-blooded animals, for example humans of approximately 70 kg body weight, is preferably from approximately 3 mg to approximately 10 g, more preferably from approximately 10 mg to approximately 1.5 g, most preferably from about 100 mg to about 1000 mg/person/day, divided preferably into 1-3 single doses which may, for example, be of the same size. Usually, children receive half of the adult dose.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes.

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8-22, especially from 12-22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, β-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gaftefossé, Paris), “Miglyol 812” (triglyceride of saturated fatty acids with a chain length of C₈-C₁₂, Hüls AG, Germany), but especially vegetable oils, such as cottonseed oil, almond oil, olive oil, castor oil, sesame oil, soybean oil and more especially groundnut oil.

The injection compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragée cores or capsules. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredient in the form of granules, for example with fillers, such as lactose, binders, such as starches, and/or glidants, such as talc or magnesium stearate, and if desired with stabilizers. In soft capsules the active ingredient is preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragée coatings or the capsule casings, for example for identification purposes or to indicate different doses of active ingredient.

Combinations

A compound of the formula (I) may also be used to advantage in combination with other antiproliferative agents. Such antiproliferative agents include, but are not limited to aromatase inhibitors; antiestrogens; topoisomerase I inhibitors; topoisomerase II inhibitors; microtubule active agents; alkylating agents; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; agents used in the treatment of hematologic malignancies; compounds which target, decrease or inhibit the activity of Flt-3; Hsp90 inhibitors; temozolomide (TEMODAL®); and leucovorin.

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in the form as it is marketed, e.g. under the trademark ARIMIDEX. Letrozole can be administered, e.g., in the form as it is marketed, e.g. under the trademark FEMARA or FEMAR. Aminoglutethimide can be administered, e.g., in the form as it is marketed, e.g. under the trademark ORIMETEN. A combination of the invention comprising a chemotherapeutic agent which is an aromatase inhibitor is particularly useful for the treatment of hormone receptor positive tumors, e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX. Raloxifene hydrochloride can be administered, e.g., in the form as it is marketed, e.g. under the trademark EVISTA. Fulvestrant can be formulated as disclosed in U.S. Pat. No. 4,659,516 or it can be administered, e.g., in the form as it is marketed, e.g. under the trademark FASLODEX. A combination of the invention comprising a chemotherapeutic agent which is an antiestrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX. Abarelix can be formulated, e.g. as disclosed in U.S. Pat. No. 5,843,901.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148 (compound A1 in WO99/17804). Irinotecan can be administered, e.g. in the form as it is marketed, e.g. under the trademark CAMPTOSAR. Topotecan can be administered, e.g., in the form as it is marketed, e.g. under the trademark HYCAMTIN.

The term “topoisomerase II inhibitor” as used herein includes, but is not limited to the an-thracyclines such as doxorubicin (including liposomal formulaton, e.g. CAELYX), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS. Teniposide can be administered, e.g. in the form as it is marketed, e.g. under the trademark VM 26-BRISTOL. Doxorubicin can be administered, e.g. in the form as it is marketed, e.g. under the trademark ADRIBLASTIN or ADRIAMYCIN. Epirubicin can be administered, e.g. in the form as it is marketed, e.g. under the trademark FARMORUBICIN. Idarubicin can be administered, e.g. in the form as it is marketed, e.g. under the trademark ZAVEDOS. Mitoxantrone can be administered, e.g. in the form as it is marketed, e.g. under the trademark NOVANTRON.

The term “microtubule active agent” relates to microtubule stabilizing, microtubule destabilizing agents and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, cochicine and epothilones and derivatives thereof, e.g. epothilone B or D or derivatives thereof. Paclitaxel may be administered e.g. in the form as it is marketed, e.g. TAXOL. Docetaxel can be administered, e.g., in the form as it is marketed, e.g. under the trademark TAXOTERE. Vinblastine sulfate can be administered, e.g., in the form as Ht is marketed, e.g. under the trademark VINBLASTIN R.P. Vincristine sulfate can be administered, e.g., in the form as it is marketed, e.g. under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Especially preferred are Epothilone A and/or B.

The term “alkylating agent” as used herein includes, but is not limited to, cyclophosphamide, ifosfamide, melphalan or nitrosourea (BCNU or Gliadel). Cyclophosphamide can be administered, e.g., in the form as it is marketed, e.g. under the trademark CYCLOSTIN. Ifosfamide can be administered, e.g., in the form as it is marketed, e.g. under the trademark HOLOXAN.

The term “histone deacetylase inhibitors” or “HDAC inhibitors” relates to compounds which inhibit the histone deacetylase and which possess antiproliferative activity. This includes compounds disclosed in WO 02/22577, especially N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes Suberoylanilide hydroxamic acid (SAHA).

The term “antineoplastic antimetabolite” includes, but is not limited to, 5-Fluorouracil or 5-FU, capecitabine, gemcitabine, DNA demethylating agents, such as 5-azacytidine and decitabine, methotrexate and edatrexate, and folic acid antagonists such as pemetrexed. Capecitabine can be administered, e.g., in the form as it is marketed, e.g. under the trademark XELODA. Gemci-tabine can be administered, e.g., in the form as it is marketed, e.g. under the trademark GEMZAR. Also included is the monoclonal antibody trastuzumab which can be administered, e.g., in the form as it is marketed, e.g. under the trademark HERCEPTIN.

The term “platin compound” as used herein includes, but is not limited to, carboplatin, cis-platin, cisplatinum and oxaliplatin. Carboplatin can be administered, e.g., in the form as it is marketed, e.g. under the trademark CARBOPLAT. Oxaliplatin can be administered, e.g., in the form as it is marketed, e.g. under the trademark ELOXATIN.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, e.g.,
a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g. a N-phenyl-2-pyrimidine-amine derivative, e.g. imatinib, SU101, SU6668 and GFB-111;
b) compounds targeting, decreasing or inhibiting the activity of the fibroblast growth factor-receptors (FGFR);
c) compounds targeting, decreasing or inhibiting the activity of the insulin-like growth factor receptor I(IGF-IR), such as compounds which target, decrease or inhibit the activity of IGF-IR, especially compounds which inhibit the IGF-IR receptor, such as those compounds disclosed in WO 02/092599;
d) compounds targeting, decreasing or inhibiting the activity of the Trk receptor tyrosine kinase family;
e) compounds targeting, decreasing or inhibiting the activity of the Axl receptor tyrosine kinase family;
f) compounds targeting, decreasing or inhibiting the activity of the Ret receptor tyrosine kinase;
g) compounds targeting, decreasing or inhibiting the activity of the Kit/SCFR receptor tyrosine kinase;
h) compounds targeting, decreasing or inhibiting the activity of the C-kit receptor tyrosine kinases—(part of the PDGFR family), such as compounds which target, decrease or inhibit the activity of the c-Kit receptor tyrosine kinase family, especially compounds which inhibit the c-Kit receptor, e.g., imatinib;
i) compounds targeting, decreasing or inhibiting the activity of members of the c-Abl family and their gene-fusion products (e.g. BCR-Abl kinase), such as compounds which target decrease or inhibit the activity of c-Abl family members and their gene fusion products, e.g. a N-phenyl-2-pyrimidine-amine derivative, e.g. imatinib; PD180970; AG957; NSC 680410; or PD173955 from ParkeDavis;
j) compounds targeting, decreasing or inhibiting the activity of members of the protein kinase C (PKC) and Raf family of serine/threonine kinases, members of the MEK, SRC, JAK, FAK, PDK and Ras/MAPK family members, or PI(3) kinase family, or of the PI(3)-kinase-related kinase family, and/or members of the cyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; limofosine; RO 318220 and RO 320432; GO 6976; Isis 3521; LY333531/LY379196; isochinoline compounds such as those disclosed in WO 00/09495; FTIs; PD184352 or QAN697 (a P13K inhibitor);
k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC) or tyrphostin. A tyrphostin is preferably a low molecular weight (Mr<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin);
l) compounds targeting, decreasing or inhibiting the activity of the epidermal growth factor family of receptor tyrosine kinases (EGFR, ErbB2, ErbB3, ErbB4 as homo- or heterodimers), such as compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (HERCEPTIN), cetuximab, Iressa, Tarceva, OSI-774, CI-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541; and
m) compounds targeting, decreasing or inhibiting the activity of the c-Met receptor.

Further anti-angiogenic compounds include compounds having another mechanism for their activity, e.g. unrelated to protein or lipid kinase inhibition e.g. thalidomide (THALOMID) and TNP-470.

Compounds which target, decrease or inhibit the activity of a protein or lipid phosphatase are e.g. inhibitors of phosphatase 1, phosphatase 2A, PTEN or CDC25, e.g. okadaic acid or a derivative thereof.

Compounds which induce cell differentiation processes are e.g. retinoic acid, α- γ- or δ-tocopherol or α- γ- or δ-tocotrienol.

The term cyclooxygenase inhibitor as used herein includes, but is not limited to, e.g. Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g. 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL. “Clodronic acid” can be administered, e.g., in the form as it is marketed, e.g. under the trademark BONEFOS. “Tiludronic acid” can be administered, e.g., in the form as it is marketed, e.g. under the trademark SKELID. “Pamidronic acid” can be administered, e.g. in the form as it is marketed, e.g. under the trademark AREDIA™. “Alendronic acid” can be administered, e.g., in the form as it is marketed, e.g. under the trademark FOSAMAX. “Ibandronic acid” can be administered, e.g., in the form as it is marketed, e.g. under the trademark BONDRANAT. “Risedronic acid” can be administered, e.g., in the form as it is marketed, e.g. under the trademark ACTONEL. “Zoledronic acid” can be administered, e.g. in the form as it is marketed, e.g. under the trademark ZOMETA.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88.

The term “biological response modifier” as used herein refers to a lymphokine or interferons, e.g. interferon γ.

The term “inhibitor of Ras oncogenic isoforms”, e.g. H-Ras, K-Ras, or N-Ras, as used herein refers to compounds which target, decrease or inhibit the oncogenic activity of Ras e.g. a “farnesyl transferase inhibitor” e.g. L-744832, DK8G557 or R115777 (Zamestra).

The term “telomerase inhibitor” as used herein refers to compounds which target, decrease or inhibit the activity of telomerase. Compounds which target, decrease or inhibit the activity of telomerase are especially compounds which inhibit the telomerase receptor, e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. PS-341 and MLN 341.

The term “matrix metalloproteinase inhibitor” or (“MMP” inhibitor) as used herein includes, but is not limited to, collagen peptidomimetic and nonpeptidomimetic Inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “agents used in the treatment of hematologic malignancies” as used herein includes, but is not limited to, FMS-like tyrosine kinase inhibitors e.g. compounds targeting, decreasing or inhibiting the activity of FMS-like tyrosine kinase receptors (Flt-3R); interferon, 1-b-D-arabinofuransylcytosine (ara-c) and bisulfan; and ALK inhibitors e.g. compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, e.g. PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90 e.g., 17-allylamino, 17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DMI, erlotinib (Tarceva), bevacizumab (Avastin™), rituximab (Rituxan®), PRO64553 (anti-CD40) and 2C4 Antibody. By antibodies is meant e.g. intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of formula (I) can be used in combination with standard leukemia therapies, especially in combination with therapies used for the treatment of AML. In particular, compounds of formula (I) can be administered in combination with, e.g., farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

The term “antileukemic compounds” includes, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate.

Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065, in particular, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt.

Compounds which target, decrease or inhibit the activity of serine/theronine mTOR kinase are especially compounds, proteins or antibodies which inhibit members of the mTOR kinase family e.g. RAD, RAD001, CCI-779, ABT578, SAR543, rapamycin and derivatives thereof; AP23573 from Ariad; everolimus (CERTICAN); and sirolimus.

Somatostatin receptor antagonists as used herein refers to agents which target, treat or inhibit the somatostatin receptor such as octreoride, and SOM230.

Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

The term EDG binders as used herein refers a class of immunosuppressants that modulates lymphocyte recirculation, such as FTY720.

CERTICAN (everolimus, RAD) an investigational novel proliferation signal inhibitor that prevents proliferation of T-cells and vascular smooth muscle cells.

The term ribonucleotide reductase inhibitors refers to pyrimidine or puring nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives, such as PL-1, PL-2, PL-3, PL-4, PL-5, PL-6, PL-7 or PL-8 mentioned in Nandy et al., Acta Oncologica, Vol. 33, No. 8, pp. 953-961 (1994).

The term “S-adenosylmethionine decarboxylase inhibitors” as used herein includes, but is not limited to the compounds disclosed in U.S. Pat. No. 5,461,076.

Also included are in particular those compounds, proteins or monoclonal antibodies of VEGF disclosed in WO 98/35958, e.g. 1-(4-chloroanilino)-4-(4-pyridylmethyl)phthalazine or a pharmaceutically acceptable salt thereof, e.g. the succinate, or in WO 00/09495, WO 00/27820, WO 00/59509, WO 98/11223, WO 00/27819 and EP 0 769 947; those as described by Prewett et al., Cancer Res, Vol. 59, pp. 5209-5218 (1999); Yuan et al., Proc Natl Acad Sci USA, Vol. 93, pp. 14765-14770 (1996); Zhu et al., Cancer Res, Vol. 58, pp. 3209-3214 (1998); and Mordenti et al., Toxicol Pathol, Vol. 27, No. 1, pp. 14-21 (1999); in WO 00/37502 and WO 94/10202; ANGIOSTATIN, described by O'Reilly et al., Cell, Vol. 79, pp. 315-328 (1994); ENDOSTATIN, described by O'Reilly et al., Cell, Vol. 88, pp. 277-285 (1997); anthranilic acid amides; ZD4190; ZD6474; SU5416; SU6668; bevacizumab; or anti-VEGF antibodies or ant-VEGF receptor antibodies, e.g. rhuMAb and RHUFab, VEGF aptamer e.g. Macugon; FLT-4 inhibitors, FLT-3 inhibitors, VEGFR-2 IgG1 antibody, Angiozyme (RPI 4610) and Avastan.

Photodynamic therapy as used herein refers to therapy which uses certain chemicals known as photosensitizing agents to treat or prevent cancers. Examples of photodynamic therapy includes treatment with agents, such as e.g. VISUDYNE and porfimer sodium.

Angiostatic steroids as used herein refers to agents which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to agents, such as e.g. fluocinolone, dexamethasone.

Other chemotherapeutic agents include, but are not limited to, plant alkaloids, hormonal agents and antagonists; biological response modifiers, preferably lymphokines or interferons; antisense oligonucleotides or oligonucleotide derivatives; or miscellaneous agents or agents with other or unknown mechanism of action.

The compounds of the invention are also useful as co-therapeutic agents for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone proplonate, cicdesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/035668, WO 03/048181, WO 03/062259, WO 03/064445, WO 03/072592, non-steroidal glucocorticoid receptor agonists such as those described in WO 00/00531, WO 02/10143, WO 03/082280, WO 03/082787, WO 03/104195, WO 04/005229;

LTB4 antagonists such LY293111, CGS025019C, CP-195543, SC-53228, BIIL 284, ONO 4057, SB 209247 and those described in U.S. Pat. No. 5,451,700; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID™ CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, NO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; A2a agonists such as those disclosed in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 04/039762, WO 04/039766, WO 04/045618 and WO 04/046083; A2b antagonists such as those described in WO 02/42298; and beta-2 adrenoceptor agonists such as albuterol (salbutamol), metaproterenol, terbutaline, salmeterol fenoterol, procaterol, and especially, formoterol and pharmaceutically acceptable salts thereof, and compounds (in free or salt or solvate form) of formula I of WO 0075114, which document is incorporated herein by reference, preferably compounds of the Examples thereof, especially a compound of formula 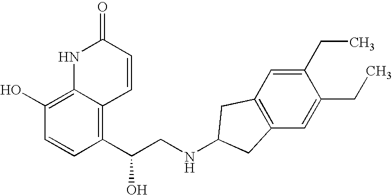
and pharmaceutically acceptable salts thereof, as well as compounds (in free or salt or solvate form) of formula I of WO 04/16601, and also compounds of WO 04/033412.

Suitable bronchodilatory drugs include anticholinergic or antimuscarinic agents, in particular ipratropium bromide, oxitropium bromide, tiotropium salts and CHF 4226 (Chiesi), and glycopyrrolate, but also those described in WO 01/04118, WO 02/51841, WO 02/53564, WO 03/00840, WO 03/87094, WO 04/05285, WO 02/00652, WO 03/53966, EP 424021, U.S. Pat. No. 5,171,744, U.S. Pat. No. 3,714,357, WO 03/33495 and WO 04/018422.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in WO 03/099807, WO 04/026841 and JP 2004107299.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-amin-ium chloride (TAK-770), and CCR-5 antagonists described in U.S. Pat. No. 6,166,037 (particularly claims 18 and 19), WO 00/66558 (particularly claim 8), WO 00/66559 (particularly claim 9), WO 04/018425 and WO 04/026873.

The structure of the active agents identified by code nos., generic or trade names may be taken from the actual edition of the standard compendium “The Merck Index” or from databases, e.g. Patents International (e.g. IMS World Publications).

The above-mentioned compounds, which can be used in combination with a compound of the formula (I), can be prepared and administered as described in the art, such as in the documents cited above.

A compound of the formula (I) may also be used to advantage in combination with known therapeutic processes, for example, the administration of hormones or especially radiation. A compound of formula (I) may in particular be used as a radiosensitizer, especially for the treatment of tumors which exhibit poor sensitivity to radiotherapy.

By “combination”, there is meant either a fixed combination in one dosage unit form, or a kit of parts for the combined administration where a compound of the formula (I) and a combination partner may be administered independently at the same time or separately within time intervals that especially allow that the combination partners show a cooperative, e.g. synergistic, effect, or any combination thereof.

### EXAMPLES

The following examples serve to illustrate the invention without limiting the scope thereof:

Abbreviations

| | |
|---|---|
| Boc | tert-butoxycarbonyl |
| conc. | concentrated |
| DMF | N,N-dimethylformamide |
| EtOAc | ethyl acetate |
| ES-MS | electrospray mass spectrometry |
| Grad | gradient |
| HPLC | high-pressure liquid |
| | chromatography |
| mL | mililitre(s) |
| m.p. | melting point |
| MS | mass spectrum |
| Prep. HPLC | preparative HPLC reverse |
| | phase C₁₈ |
| sat. | saturated |
| RT | room temperature |
| tᵣₑₜ | HPLC retention time in |
| | minutes |
| TFA | trifluoroacetic acid |
| THF | tetrahydrofuran |

Where no temperature values are given, the reaction takes place at ambient (room) temperature.

Ratios of solvents (e.g. in eluents or solvent mixtures) are given in volume by volume (v/v).

HPLC linear gradient between A=H₂O/TFA 1000:1 and B=acetonitrile/TFA 1000:1

Grad 1: 20-100% B in 5 minutes and 1.5 minutes at 100% B, column: Nucleosil 100-3 C₁₈ reverse phase, 70 mm×4 mm, particle size 3 μm, Å (Macherey & Nagel, Düren, Germany); flow rate: 1.25 ml/min.; detection at 215 nM.

Grad 2: 2-100% B in 4.5 minutes and 1 minute at 100% B, column: Chromolith Performance 100 mm×4.5 mm (Merck, Darmstadt, Germany); flow rate 2 mL/min.; detection at 215 nM.

Grad 3: 2-100% B in 7 minutes and 3 minutes at 100% B; column: Nucleosil C₁₈ reverse phase; 250 mm×4.6 mm (SMT, Burkard Instruments, Dietikon, Switzerland); particle size 5 μm, 100 Å; flow rate: 2.0 mL/min.; detection at 215 nm.

### Example 1

### 2-[4-(8-Phenylethynyl-imidazo[4,5-c]quinolin-1-yl)-phenyl]-ethylamine

74 mg (0.151 mmol) of (2-[4-(8-phenylethynyl-imidazo[4,5-c]quinolin-1-yl)-phenyl]-ethyl)carbamic acid tert-butyl ester (Example 1h) are dissolved in 2 mL of TFA-H₂O (19:1 or 1:1) and the progress of the reaction is monitored by analytical HPLC. After complete removal of the Boc protecting group, the solvent is evaporated to dryness and the residue purified by prep. HPLC. The pure fractions are condensed, basified with NaHCO₃ and extracted with ethyl acetate (3×). The organic layers are dried over MgSO₄, filtered and evaporated to dryness to give 2-[4-(8-phenylethynyl-imidazo[4,5-c]quinolin-1-yl)-phenyl]-ethylamine as an off-white solid: ES-MS: 389 (M+H)⁺; analytical HPLC: tᵣₑₜ=2.98 minutes (Grad 1).

### Example 1a

### 5-Bromo-2-(2-nitro-vinylamino)-benzoic acid

A suspension of 25 g (16 mmol) of 2-amino-5-bromo-benzoic acid (Fluka, Buchs, Switzerland) in H₂O—HCl (37%) (10:1) is stirred for 8 hours and then filtered (Solution A). 8.17 g (255 mmol) of nitromethane (Fluka, Buchs, Switzerland) are added over 10 minutes to an ice-bath cooled mixture of 35 g of ice and 15.3 g (382 mmol) of NaOH. After stirring for 1 hour at 0° C. and 1 hour at RT, the solution is added at 0° C. to 28 g of ice and 42 mL of HCl (37%) (Solution B). Solutions A and B are combined and the reaction mixture is stirred for 18 hours at RT. The yellow precipitate is filtered off and washed with H₂O. 5-Bromo-2-(2-nitrovinylamino)-benzoic acid is dried in vacuo at 40° C. ES-MS: 287, 289 (M+H)⁺, Br pattern.

¹H NMR (DMSO-d₆): δ 13.7-14.6 (br, s, 1H), 12.94 (d, 1H), 8.07 (d, 1H), 8.03 (dd, 1H), 7.83 (dd, 1H), 7.71 (d, 1H), 6.76 (d, 1H); analytical HPLC: tᵣₑₜ=3.93 minutes (Grad 1).

### Example 1b

### 6-Bromo-3-nitro-quinolin-4-ol

29 g (101 mmol) of 5-bromo-2-(2-nitro-vinylamino)-benzoic acid (Example 1a) and 11.9 g (121 mmol) of potassium acetate in 129 mL (152 mmol) of acetic anhydride are stirred for 1.5 hours at 120° C. The precipitate is filtered-off and washed with acetic acid until the filtrate is colorless and then with H₂O. 6-Bromo-3-nitro-quinolin-4-ol is dried in vacuo. ES-MS: 269, 271 (M+H)⁺, Br pattern; analytical HPLC: tᵣₑₜ=3.01 minutes (Grad 1).

### Example 1c

### 6-Bromo-4-chloro-3-nitro-quinoline

7.8 g (29 mmol) of 6-bromo-3-nitro-quinolinol (Example 1b) in 58 mL (230 mmol) of POCl₃ are stirred for 2 hours at 120° C. The mixture is cooled to rt and poured slowly into ice-water. The precipitate is filtered-off, washed with ice-cold water, and dissolved in CH₂Cl₂. The organic phase is washed with cold brine, and the aqueous phase is discarded. After drying over MgSO₄, the organic solvent is evaporated to dryness to provide 6-bromo-4-chloro-3-nitro-quinoline.

¹H NMR (CDCl₃): δ 9.20 (s, 1H), 8.54 (d, 1H), 8.04 (d, 1H), 7.96 (dd, 1H); analytical HPLC: tᵣₑₜ=4.32 minutes (Grad 2).

### Example 1d

### [2-(4-Amino-phenyl)-ethyl]-carbamic acid tert-butyl ester

[2-(4-Amino-phenyl)-ethyl]-carbamic acid tert-butyl ester is obtained as described in J Med Chem, Vol. 35, p. 4264 (1992); ES-MS: 237 (M+H)⁺; analytical HPLC: tᵣₑₜ=2.54 minutes (Grad 2).

### Example 1e

### {2-[4-(6-Bromo-3-nitro-quinolin-4-ylamino)-phenyl]-ethyl}-carbamic acid tert-butyl ester

0.66 g (2.31 mmol) of 6-bromo-4-chloro-3-nitro-quinoline (Example 1c) and 0.60 g (2.54 mmol) of [2-(4-amino-phenyl)-ethyl]-carbamic acid tert-butyl ester (Example 1d) are dissolved in 7 mL of acetic acid and stirred for 1 hour. After this time, water is added and the yellow precipitate is filtered-off and washed with H₂O. The solid is dissolved in EtOAc-THF (3:1), washed with aqueous NaHCO₃ and brine and dried over MgSO₄. The organic phase is evaporated to dryness to give {2-[4-(6-bromo-3-nitro-quinolin-4-ylamino)-phenyl]-ethyl}-carbamic acid tert-butyl ester as a yellow solid. ES-MS: 487, 489 (M+H)⁺, Br pattern; analytical HPLC: tᵣₑₜ=3.92 minutes (Grad 2).

### Example 1f

### {2-[4-(3-Amino-6-bromo-quinolin-4-ylamino)-phenyl]-ethyl}-carbamic acid tert-butyl ester

1.1 g (2.26 mmol) of {2-[4-(6-bromo-3-nitro-quinolin-4-ylamino)-phenyl]-ethyl}-carbamic acid tert-butyl ester (Example 1e) is shacked in 26 mL of MeOH-THF (2:1) under 1.1 bar of H₂ in the presence of 0.5 g of Raney-Ni for 3 hours. After completion of the reaction, the catalyst is filtered-off and the filtrate is evaporated to dryness to give {2-[4-(3-amino-6-bromo-quinolin-4-ylamino)-phenyl]-ethyl}-carbamic acid tert-butyl ester as a yellow foam. ES-MS: 457, 459 (M+H)⁺, Br pattern; analytical HPLC: tᵣₑₜ=3.41 minutes (Grad 2).

### Example 1g

### {2-[4-(8-Bromo-imidazo[4,5-c]quinolin-1-yl)-phenyl]-ethyl}-carbamic acid tert-butyl ester

1.03 g (2.26 mmol) of {2-[4-(3-amino-6-bromo-quinolin-4-ylamino)-phenyl]-ethyl}-carbamic add tert-butyl ester in 30 mL triethylorthoformate is heated for 2 hours at 105° C., and then evaporated in vacuo to dryness. The residue is purified by flash chromatography on silica gel (CH₂Cl₂-MeOH 3:197 to 1:24) to provide {2-[4-(8-bromo-imidazo[4,5-c]quinolin-1-yl)-phenyl]-ethyl}-carbamic acid tert-butyl ester as a pink foam. ES-MS: 467, 469 (M+H)⁺, Br pattern; analytical HPLC: tᵣₑₜ=3.36 minutes (Grad 2).

### Example 1h

### {2-[4-(8-Phenylethynyl-imidazo[4,5-c]quinolin-1-yl)-phenyl]-ethyl}-carbamic acid tert-butyl ester

To 80 mg (0.171 mmol) of {2-[4-(8-bromo-imidazo[4,5-c]quinolin-1-yl)-phenyl]-ethyl}-carbamic acid tert-butyl ester (Example 1g), 1 mg (0.0053 mmol) of CuI and 3 mg (0.0078 mmol) of bis(benzonitrile)palladium (II) chloride in 0.25 mL of dioxane under an argon atmosphere are added 21 mg (0.205 mmol) of phenylacetylene (Fluka, Buchs, Switzerland), 0.05 mL (0.012 mmol) of 0.25 M tri-tert-butylphosphine in dioxane and 20.3 mg (0.205 mmol) of diisopropylamine. The reaction mixture is stirred for 2 hours, and then quenched with aqueous sat. NaHCO₃ and extracted with EtOAc. The organic layer is washed with brine, dried over MgSO₄, filtered and evaporated in vacuo. The residue is purified by flash chromatography on silica gel (CH₂Cl₂-MeOH 99:1 to 193:7) to give {2-[4-(8-phenylethynyl-imidazo[4,5c]quinolin-1-yl)-phenyl]-ethyl}-carbamic acid tert-butyl ester as an oil. ES-MS: 489 (M+H)⁺; analytical HPLC: tᵣₑₜ=4.76 minutes (Grad 1).

The following compounds (see Table 1) are prepared as described in Example 1 by reacting {2-[4-(8-bromo-imidazo[4,5-c]quinolin-1-yl)-phenyl]-ethyl}-carbamic acid tert-butyl ester (Example 1g), with the appropriate alkyne as shown in Example 1h.

Example 2 3-methoxyphenylacetylene (Fluka, Buchs, Switzerland);

Example 3 4-methoxyphenylacetylene (Fluka, Bucks, Switzerland);

Example 4 3-ethynylpyridine (Aldrich, Buchs, Switzerland);

Example 5 5-ethynyl-2-methoxy-pyridine (Example 5a);

Example 6 5-ethynyl-benzo[1,3]dioxole (Example 6a); and

Example 7 4-ethynyl-benzenesulfonamide (Example 7a).

TABLE 1

| | | ES-MS | tᵣₑₜ |
|---|---|---|---|
| Example | Compound name | (M + H)⁺ | [min] |
| 2 | 2-{4-[8-(3-Methoxy-phenylethynyl)-imidazo[4,5-c]quinolin- | 419 | 3.07 |
| | 1-yl]-phenyl}-ethylamine | | Grad 1 |
| 3 | 2-{4-[8-(4-Methoxy-phenylethynyl)-imidazo[4,5-c]quinolin- | 419 | 3.01 |
| | 1-yl]-phenyl}-ethylamine | | Grad 1 |
| 4 | 2-[4-(8-Pyridin-3-ylethynyl-imidazo[4,5-c]quinolin-1-yl)- | 390 | 1.95 |
| | phenyl]-ethylamine | | Grad 1 |
| 5 | 2-{4-[8-(6-Methoxy-pyridin-3-ylethynyl)-imidazo[4,5- | 420 | 2.60 |
| | c]quinolin-1-yl]-phenyl}-ethylamine | | Grad 2 |
| 6 | 2-[4-(8-Benzo[1,3]dioxol-5-ylethynyl-imidazo[4,5- | 433 | 3.01 |
| | c]quinolin-1-yl)-phenyl]-ethylamine | | Grad 1 |
| 7 | 4-{1-[4-(2-Amino-ethyl)-phenyl]-1H-imidazo[4,5-c]quinolin- | 468 | 2.54 |
| | 8-ylethynyl}-benzenesulfonamide | | Grad 1 |

### Example 5a

### 5-Ethynyl-2-methoxy-pyridine

To a cold solution of 2.2 g (10.6 mmol) of 2-methoxy-5-trimethylsilanylethynyl-pyridine (Example 5b) in 25 mL of THF is slowly added a solution of 3.68 g (11.7 mmol) of tetrabutylammonium fluoride trihydrate in 5 mL of H₂O. The reaction mixture is stirred for 1 hour. After this time, the reaction mixture is treated with aqueous sat. NaHCO₃ and extracted with CH₂Cl₂. The organic layer is washed with aqueous sat. NaHCO₃ and brine, dried over MgSO₄, filtered and evaporated to dryness. The residue is purified by flash chromatography on silica gel (hexane/EtOAc (20:1) to (10:1)) to give a brown oil. 5-Ethynyl-2-methoxy-pyridine is obtained by bulb to bulb distillation at reduced pressure as a colorless liquid. ES-MS: 134 (M+H)⁺; analytical HPLC: tᵣₑₜ=3.39 minutes (Grad 2).

### Example 5b

### 2-Methoxy-5-trimethylsilanylethynyl-pyridine

To 41 mg (0.213 mmol) of CuI and 122 mg (0.319 mmol) of bis(benzonitrile)palladium (II) chloride in 10 mL of dioxane are added under an argon atmosphere 2 g (10.6 mmol) of 5-bromo-2-methoxy-pyridine (Aldrich, Buchs, Switzerland), 1.25 g (12.8 mmol) of trimethylsilylacetylene (Fluka, Buchs, Switzerland), 2.55 mL (0.638 mmol) of 0.25 M tri-tert-butylphosphine in dioxane and 1.3 g (12.8 mmol) of diisopropylamine. The reaction mixture is stirred for 12 hours. After this time, the reaction mixture is treated with aqueous sat. NaHCO₃ and extracted with EtOAc. The organic layer is washed with brine, dried over MgSO₄, filtered and evaporated to dryness. The residue is purified by flash chromatography on silica gel (hexane-EtOAc (20:1) to (10:1)) to provide 2-Methoxy-5-trimethylsilanylethynyl-pyridine as a brown oil. ES-MS: 206 (M+H)⁺; analytical HPLC: tᵣₑₜ=4.85 minutes (Grad 2).

### Example 6a

### 5-Ethynyl-benzo[1,3]dioxole

5-Ethynyl-benzo[1,3]dioxole is obtained as described in Example 5a using 5-bromo-benzo[1,3]-dioxole (Fluka, Buchs, Switzerland) instead of 5-bromo-2-methoxypyridine, analytical HPLC: tᵣₑₜ=3.71 minutes (Grad 2).

### Example 7a

### 4-Ethynyl-benzenesulfonamide

4-Ethynyl-benzenesulfonamide is obtained as described in Example 5a using 4-bromo-benzenesulfonamide (Fluka, Buchs, Switzerland) instead of 5-bromo-2-methoyxpyridine. ES-MS: 180 (M−H)⁺; analytical HPLC: tᵣₑₜ=2.65 minutes (Grad 2).

The following compounds (see Table 2) are prepared as described in Example 1 using [3-(4-amino-phenyl)-propyl]-carbamic acid tert-butyl ester (Example 8a) and the appropriate alkyne according to Example 1h.

Example 8 phenylacetylene (Fluka, Buchs, Switzerland);

Example 9 3-methoxyphenylacetylene (Fluka, Buchs, Switzerland);

Example 10 4-methoxyphenylacetylene (Fluka, Bucks, Switzerland);

Example 11 3-ethynylpyridine (Aldrich, Buchs, Switzerland);

Example 12 5-ethynyl-benzo[1,3]dioxole (Example 6a); and

Example 13 4-ethynyl-benzenesulfonamide (Example 7a).

TABLE 2

| | | ES-MS | tᵣₑₜ |
|---|---|---|---|
| Example | Compound name | (M + H)⁺ | [min] |
| 8 | 3-[4-(8-Phenylethynyl-imidazo[4,5-c]quinolin-1-yl)- | 403 | 3.10 |
| | phenyl]-propylamine | | Grad 1 |
| 9 | 3-{4-[8-(3-Methoxy-phenylethynyl)-imidazo[4,5- | 433 | 3.18 |
| | c]quinolin-1-yl]-phenyl}-propylamine | | Grad 1 |
| 10 | 3-{4-[8-(4-Methoxy-phenylethynyl)-imidazo[4,5- | 433 | 3.18 |
| | c]quinolin-1-yl]-phenyl}-propylamine | | Grad 1 |
| 11 | 3-[4-(8-Pyridin-3-ylethynyl-imidazo[4,5-c]quinolin-1-yl)- | 404 | 2.27 |
| | phenyl]-propylamine | | Grad 1 |
| 12 | 3-[4-(8-Benzo[1,3]dioxol-5-ylethynyl-imidazo[4,5- | 447 | 3.13 |
| | c]quinolin-1-yl)-phenyl]-propylamine | | Grad 1 |
| 13 | 4-{1-[4-(3-Amino-propyl)-phenyl]-1H-imidazo[4,5- | 482 | 2.66 |
| | c]quinolin-8-ylethynyl}-benzenesulfonamide | | Grad 1 |

### Example 8a

### [3-(4-Amino-phenyl)-propyl]-carbamic acid tert-butyl ester

2 g (11.4 mmol) of 3-(4-nitro-phenyl)-propionitrile (Example 8b) and 0.5 g of Raney-Ni are shacked in 40 mL of THF-[MeOH/NH₃ (5%)] (1:1) under 1.1 bar of H₂ for 36 hours at 44° C. After completion of the reaction, the catalyst is filtered-off and the filtrate is evaporated in vacuo. The residue is dissolved in 20 mL of THF and 15 mL of aqueous sat. NaHCO₃. The solution is cooled with an ice-bath and 2.23 g (10.2 mmol) of (Boc)₂O (Fluka, Buchs, Switzerland) in 10 mL of THF are added over 1 hour. The reaction mixture is stirred for 1.5 hours at RT, is diluted with water and extracted with EtOAc. The organic layer is washed with 10% of citric acid, sat. NaHCO₃ and brine, dried over MgSO₄, filtered and evaporated. The residue is purified by flash chromatography on silica gel (hexane-EtOAc, 2:1 to 1:1) to provide [3-(4-amino-phenyl)-propyl]-carbamic acid tert-butyl ester as an oil. ES-MS: 251 (M+H)⁺; analytical HPLC: tᵣₑₜ=2.85 minutes (Grad 1).

### Example 8b

### 3-(4-Nitro-phenyl)-propionitrile

10.12 g (44 mmol) of 1-(2-bromo-ethyl)-4-nitro-benzene (Aldrich, Buchs, Switzerland) and 2.16 g (44 mmol) of NaCN in 110 mL of ethanol are refluxed for 16 hours. The reaction mixture is evaporated in vacuo and purified by flash chromatography on silica gel (CH₂Cl₂) to provide 3-(4-nitro-phenyl)-propionitrile as an off-white solid.

¹H NMR (DMSO-d₆): δ 8.23 (m, 2H), 7.62 (m, 2H), 3.06 (d, 2H), 2.92 (d, 1H); analytical HPLC: tᵣₑₜ=3.83 minutes (Grad 1).

The following compounds (see Table 3) are synthesized as described in Example 1 using 6-bromo-4,7-dichloro-3-nitro-quinoline in Example 1c, which is obtained in analogy to 6-bromo-4-chloro-3-nitro-quinoline (Example 1c) and starting from 2-amino-5-bromo-4-chloro-benzoic acid (Example 14a) in Example 1a, and the required alkyne in Example 1h.

Example 14 phenylacetylene (Fluka, Buchs, Switzerland);

Example 15 3-methoxyphenylacetylene (Fluka, Buchs, Switzerland);

Example 16 4-methoxyphenylacetylene (Fluka, Bucks, Switzerland);

Example 17 3-ethynylpyridine (Aldrich, Buchs, Switzerland);

Example 18 5-ethynyl-benzo[1,3]dioxole (Example 6a); and

Example 19 4-ethynyl-benzenesulfonamide (Example 7a).

TABLE 3

| | | ES-MS | tᵣₑₜ |
|---|---|---|---|
| Example | Compound name | (M + H)⁺ | [min] |
| 14 | 2-[4-(7-Chloro-8-phenylethynyl-imidazo[4,5-c]quinolin- | 423 | 3.55 |
| | 1-yl)-phenyl]-ethylamine | | Grad 1 |
| 15 | 2-{4-[7-Chloro-8-(3-methoxy-phenylethynyl)- | 453 | 3.62 |
| | imidazo[4,5-c]quinolin-1-yl]-phenyl}-ethylamine | | Grad 1 |
| 16 | 2-{4-[7-Chloro-8-(4-methoxy-phenylethynyl)- | 453 | 3.59 |
| | imidazo[4,5-c]quinolin-1-yl]-phenyl}-ethylamine | | Grad 1 |
| 17 | 2-[4-(7-Chloro-8-pyridin-3-ylethynyl-imidazo[4,5- | 424 | 2.79 |
| | c]quinolin-1-yl)-phenyl]-ethylamine | | Grad 1 |
| 18 | 2-[4-(7-Chloro-8-benzo[1,3]dioxol-5-ylethynyl- | 467 | 3.57 |
| | imidazo[4,5-c]quinolin-1-yl)-phenyl]-ethylamine | | Grad 1 |
| 19 | 4-{1-[4-(2-Amino-ethyl)-phenyl]-7-chloro-1H- | 502 | 3.06 |
| | imidazo[4,5-c]quinolin-8-ylethynyl}- | | Grad 1 |
| | benzenesulfonamide | | |

### Example 14a

### 2-Amino-5-bromo-4-chloro-benzoic acid

34.2 g (200 mmol) of 2-aminochlorobenzoic acid (Fluka, Buchs, Switzerland) are dissolved in 1900 mL of methanol and the solution is cooled at −70° C. To this stirred solution, 11.2 mL (218 mmol) of bromine dissolved in 110 mL of methanol are added slowly. After 3 hours, the solution is added to ice-water and the aqueous phase is extracted with ether. The combined organic portions are washed with water, brine, dried over MgSO₄ and concentrated in vacuo to provide 2-amino-5-bromo-4-chloro-benzoic acid. 2-amino-5-bromo-4-chloro-benzoic acid, m.p. 228-230° C.

¹H NMR (DMSO-d₆): δ 7.85 (s, 1H), 6.95 (s, 1H).

The following compounds (see Table 4) are synthesized as described in Example 1 starting from 6-bromo-4,7-dichloro-3-nitro-quinoline in Example 1c, [3-(4-amino-phenyl)-propyl]-carbamic acid tert-butyl ester (Example 8a) in Example 1d and the required alkyne in Example 1h.

Example 20 phenylacetylene (Fluka, Buchs, Switzerland);

Example 21 3-methoxyphenylacetylene (Fluka, Buchs, Switzerland);

Example 22 4-methoxyphenylacetylene (Fluka, Bucks, Switzerland);

Example 23 3-ethynylpyridine (Aldrich, Buchs, Switzerland);

Example 24 5-ethynyl-benzo[1,3]dioxole (Example 6a); and

Example 25 4-ethynyl-benzenesulfonamide (Example 7a).

TABLE 4

| | | ES-MS | tᵣₑₜ |
|---|---|---|---|
| Example | Compound name | (M + H)⁺ | [min] |
| 20 | 3-[4-(7-Chloro-8-phenylethynyl-imidazo[4,5-c]quinolin- | 437 | 3.72 |
| | 1-yl)-phenyl]-propylamine | | Grad 1 |
| 21 | 3-{4-[7-Chloro-8-(3-methoxy-phenylethynyl)- | 467 | 3.80 |
| | imidazo[4,5-c]quinolin-1-yl]-phenyl}-propylamine | | Grad 1 |
| 22 | 3-{4-[7-Chloro-8-(4-methoxy-phenylethynyl)- | 467 | 3.76 |
| | imidazo[4,5-c]quinolin-1-yl]-phenyl}-propylamine | | Grad 1 |
| 23 | 3-[4-(7-Chloro-8-pyridin-3-ylethynyl-imidazo[4,5- | 438 | 2.96 |
| | c]quinolin-1-yl)-phenyl]-propylamine | | Grad 1 |
| 24 | 3-[4-(7-Chloro-8-benzo[1,3]dioxol-5-ylethynyl- | 481 | 3.72 |
| | imidazo[4,5-c]quinolin-1-yl)-phenyl]-propylamine | | Grad 1 |
| 25 | 4-{1-[4-(3-Amino-propyl)-phenyl]-7-chloro-1H- | 516 | 3.20 |
| | imidazo[4,5-c]quinolin-8-ylethynyl}- | | Grad 1 |
| | benzenesulfonamide | | |

The following compounds (see Table 5) are synthesized as described in Example 1 using 6-bromo-4-chloro-7-fluoro-3-nitro-quinoline in Example 1c, which is obtained in analogy to 6-bromo-4-chloro-3-nitro-quinoline (Example 1c) and starting from 2-amino-5-bromo-4-fluoro-benzoic acid (Example 26a) in Example 1a, and the required alkyne in Example 1h.

Example 26 phenylacetylene (Fluka, Buchs, Switzerland);

Example 27 3-methoxyphenylacetylene (Fluka, Buchs, Switzerland);

Example 28 4-methoxyphenylacetylene (Fluka, Bucks, Switzerland);

Example 29 3-ethynylpyridine (Aldrich, Buchs, Switzerland);

Example 30 5-ethynyl-benzo[1,3]dioxole (Example 6a); and

Example 31 4-ethynyl-benzenesulfonamide (Example 7a).

TABLE 5

| | | ES-MS | tᵣₑₜ |
|---|---|---|---|
| Example | Compound name | (M + H)⁺ | [min] |
| 26 | 2-[4-(7-Fluoro-8-phenylethynyl-imidazo[4,5-c]quinolin- | 407 | 3.24 |
| | 1-yl)-phenyl]-ethylamine | | Grad 1 |
| 27 | 2-{4-[7-Fluoro-8-(3-methoxy-phenylethynyl)- | 437 | 3.31 |
| | imidazo[4,5-c]quinolin-1-yl]-phenyl}-ethylamine | | Grad 1 |
| 28 | 2-{4-[7-Fluoro-8-(4-methoxy-phenylethynyl)- | 437 | 3.29 |
| | imidazo[4,5-c]quinolin-1-yl]-phenyl}-ethylamine | | Grad 1 |
| 29 | 2-[4-(7-Fluoro-8-pyridin-3-ylethynyl-imidazo[4,5- | 408 | 2.46 |
| | c]quinolin-1-yl)-phenyl]-ethylamine | | Grad 1 |
| 30 | 2-[4-(7-Fluoro-8-benzo[1,3]dioxol-5-ylethynyl- | 451 | 3.26 |
| | imidazo[4,5-c]quinolin-1-yl)-phenyl]-ethylamine | | Grad 1 |
| 31 | 4-{1-[4-(2-Amino-ethyl)-phenyl]-7-fluoro-1H- | 486 | 2.81 |
| | imidazo[4,5-c]quinolin-8-ylethynyl}- | | Grad 1 |
| | benzenesulfonamide | | |

### Example 26a

### 2-Amino-5-bromo-4-fluoro-benzoic acid

2-Amino-5-bromo-4-fluoro-benzoic acid is obtained as described in Example 14a starting with 2-amino-4-fluorobenzoic acid (Fluka, Buchs, Switzerland). 2-Amino-5-bromo-4-fluoro-benzoic acid; m.p. 216-218° C.

¹H NMR (DMSO-d₆): δ 7.85 (d, 1H), 6.64 (d, 1H).

The following compounds (see Table 6) are synthesized as described in Example 1 using triethyl orthoacetate (Fluka, Buchs, Switzerland), triethyl orthopropionate (Fluka, Buchs, Switzerland) or trimethyl orthobutyrate (Fluka, Buchs, Switzerland) in Example 1g, and the required alkyne in Example 1h.

TABLE 6

| | | ES-MS | tᵣₑₜ |
|---|---|---|---|
| Example | Compound name | (M + H)⁺ | [min] |
| 32 | 2-[4-(2-Methyl-8-phenylethynyl-imidazo[4,5-c]quinolin- | 403 | 2.91 |
| | 1-yl)-phenyl]-ethylamine | | Grad 1 |
| 33 | 2-{4-[8-(3-Methoxy-phenylethynyl)-2-methyl- | 433 | 2.98 |
| | imidazo[4,5-c]quinolin-1-yl]-phenyl}-ethylamine | | Grad 1 |
| 34 | 2-{4-[8-(4-Methoxy-phenylethynyl)-2-methyl- | 433 | 2.99 |
| | imidazo[4,5-c]quinolin-1-yl]-phenyl}-ethylamine | | Grad 1 |
| 35 | 2-[4-(2-Methyl-8-pyridin-3-ylethynyl-imidazo[4,5- | 404 | 1.90 |
| | c]quinolin-1-yl)-phenyl]-ethylamine | | Grad 1 |
| 36 | 2-[4-(2-Ethyl-8-pyridin-3-ylethynyl-imidazo[4,5- | 418 | 2.22 |
| | c]quinolin-1-yl)-phenyl]-ethylamine | | Grad 2 |
| 37 | 2-[4-(3-Propyl-8-pyridin-3-ylethynyl-imidazo[4,5- | 432 | 2.31 |
| | c]quinolin-1-yl)-phenyl]-ethylamine | | Grad 2 |

### Example 38

### 3-[4-(8-trans-Styryl-imidazo[4,5-c]quinolin-1-yl)-phenyl]-propylamine

71 mg (0.141 mmol) of {3-[4-(8-trans-styryl-imidazo[4,5-c]quinolin-1-yl)-phenyl]-propyl}-carbamic acid tert-butyl ester (Example 38b) in 2 mL of TFA-H₂O (19:1) are stirred for 10 minutes. The solvent is evaporated to dryness and the residue purified by prep. HPLC. The pure fractions are condensed, basified with NaHCO₃ and extracted with ethyl acetate (3×). The organic layers are dried over MgSO₄, filtered and evaporated to dryness to give 3-[4-(8-trans-styryl-imidazo[4,5-c]quinolin-1-yl)-phenyl]-propylamine as an off-white solid; ES-MS: 405 (M+H)⁺; analytical HPLC: tᵣₑₜ=3.00 minutes (Grad 1).

### Example 38b

### {3-[4-(8-trans-Styryl-imidazo[4,5-c]quinolin-1-yl)-phenyl]-propyl}-carbamic acid tert-butyl ester

70 mg (0.145 mmol) of {3-[4-(8-bromo-imidazo[4,5c]quinolin-1-yl)-phenyl]-propyl}-carbamic acid tert-butyl ester (intermediate for the synthesis of Example 8), 32 mg (0.218 mmol) of trans-phenylethenylboronic acid (Aldrich, Buchs, Switzerland) and 6 mg (0.009 mmol) of bis(triphenylphosphino)palladium(II) chloride in 1.8 mL DMF and 0.364 mL (0.364 mmol) of 1 M aqueous potassium carbonate are stirred for 1 hour at 100° C. under an argon atmosphere. After this time, the reaction mixture is cooled down to RT and is treated with aqueous sat. NaHCO₃ and extracted with EtOAc (2×). The combined organic layer are washed with brine (3×), dried over MgSO₄, filtered and evaporated to dryness. The residue is purified by flash chromatography on silica gel (CH₂Cl₂-MeOH (99:1) to (193:7)) to give {3-[4-(8-trans-styryl-imidazo[4,5c]quinolin-1-yl)-phenyl]-propyl}-carbamic acid tert-butyl ester as a solid. ES-MS: 505 (M+H)⁺; analytical HPLC: tᵣₑₜ=4.71 minutes (Grad 1).

The following compounds (see Table 7) are synthesized as described in Example 38 starting from {3-[4-(8-bromo-7-chloro-imidazo[4,5-c]quinolin-1-yl)-phenyl]-propyl}-carbamic acid tert-butyl ester (intermediate in the synthesis of Example 14, i.e. the result of Step 1g in Example 14) or {2-[4-(8-bromo-7-chloro-imidazo[4,5-c]quinolin-1-yl)-phenyl]-ethyl}-carbamic acid tert-butyl ester (intermediate in the synthesis of Example 20, i.e. the result of Step 1g in Example 20).

TABLE 7

| | | ES-MS | |
|---|---|---|---|
| Example | Compound name | (M + H)⁺ | tᵣₑₜ [min] |
| 39 | 2-[4-(7-Chloro-8-styryl-imidazo[4,5- | 425 | 3.32 |
| | c]quinolin-1-yl)-phenyl]- | | Grad 1 |
| | ethylamine | | |
| 40 | 3-[4-(7-Chloro-8-styryl-imidazo[4,5- | 439 | 3.44 |
| | c]quinolin-1-yl)-phenyl]- | | Grad 1 |
| | propylamine | | |

The following compounds (see Table 8) are prepared as described in Example 1 by reacting {2-[4-(8-bromo-imidazo[4,5-c]quinolin-1-yl)-phenyl]-ethyl}-carbamic acid tert-butyl ester (Example 1g), with the required alkyne as shown in Example 1h.

Example 41 5-Ethynyl-2-fluoro-pyridine (Example 41a);

Example 42 4-(5-Ethynyl-pyridin-2-yl)-morpholine (Example 42a); and

Example 43 (5-Ethynyl-pyridin-2-yl)-dimethyl-amine (Example 43a).

TABLE 8

| | | ES-MS | tᵣₑₜ |
|---|---|---|---|
| Example | Compound name | (M + H)⁺ | [min] |
| 41 | 2-{4-[8-(6-Fluoro-pyridin-3-ylethynyl)- | 408 | 2.52 |
| | imidazo[4,5-c]quinolin-1-yl]- | | Grad 2 |
| | phenyl}-ethylamine | | |
| 42 | 2-{4-[8-(6-Morpholin-4-yl-pyridin-3- | 475 | 2.36 |
| | ylethynyl)-imidazo[4,5-c]quinolin-1-yl]- | | Grad 2 |
| | phenyl}-ethylamine | | |
| 43 | (5-{1-[4-(2-Amino-ethyl)-phenyl]-1H- | 433 | 2.17 |
| | imidazo[4,5-c]quinolin-8-ylethynyl}- | | Grad 2 |
| | pyridin-2-yl)-dimethyl-amine | | |

### Example 41a

### 5-Ethynyl-2-fluoro-pyridine

The title compound is obtained as described in Example 5a using 5-bromo-2-fluoropyridine (Aldrich, Buchs, Switzerland) instead of 5-bromo-2-methoxypyridine. Analytical HPLC: tᵣₑₜ=3.03 minutes (Grad 2).

### Example 42a

### 4-(5-Ethynyl-pyridin-2-yl)-morpholine

The title compound is obtained as described in Example 5a using 4-(5-bromo-pyridin-2-yl)-morpholine (Example 42b) instead of 5-bromo-2-methoxypyridine. ES-MS: 189 (M+H)⁺; analytical HPLC: tᵣₑₜ=2.09 minutes (Grad 2).

### Example 42b

### 4-(5-Bromo-pyridin-2-yl)-morpholine

3.0 g (12.7 mmol) of 2,5-dibromopyridine (Aldrich, Buchs, Switzerland) are suspended in 15.0 ml (172 mmol) of morpholine. The mixture is heated in a microwave for 100 min at 120° C. After this time, 150 ml of ethylacetate are added and the solution is washed with 0.1 N hydrochloric acid, water, 0.1 N NaOH, and water. The organic phase is evaporated to dryness to provide the title compound; ES-MS: 243 (M+H)⁺.

### Example 43a

### (5-Ethynyl-pyridin-2-yl)-dimethyl-amine

The title compound is obtained as described in Example 5a using (5-bromo-pyridin-2-yl)-dimethyl-amine (Example 43b) instead of 5-bromo-2-methoxypyridine. ES-MS: 147 (M+H)⁺; analytical HPLC: tᵣₑₜ=1.90 minutes (Grad 2).

### Example 43b

### 4-(5-Bromo-pyridin-2-yl)-dimethyl-amine

The title compound is obtained as described in Example 42b using diethanolamine (Fluka, Buchs, CH) instead of morpholine. ES-MS: 201, 203 (M+H)⁺, Br pattern; analytical HPLC: tᵣₑₜ=1.94 minutes (Grad 2).

### Example 44

### 3-[4-(2-Methyl-8-pyridin-3-ylethynyl-imidazo[4,5-c]quinolin-1-yl)-phenyl]-propylamine

The title compound is obtained as described in Example 1 using [3-(4-amino-phenyl)-propyl]-carbamic acid tert-butyl ester (Example 8a) as in Example 1e and triethyl orthoacetate as described in Example 1g. ES-MS: 418 (M+H)⁺; analytical HPLC: tᵣₑₜ=2.28 minutes (Grad 2).

The following compounds (see Table 9) are synthesized as described in Example 1 with an alternative cyclisation of {2-[4-(3-amino-6-bromo-quinolin-4-ylamino)phenyl]-ethyl}-carbamic acid tert-butyl ester (Example 1f) using tetramethylothocarbonate (Aldrich, Buchs, Switzerland) (Example 45a), cyclopropanecarboxaldehyde (Aldrich, Buchs, Switzerland) (Example 46a) or isobuyraldehyde (Aldrich, Buchs, Switzerland) (Example 47a).

TABLE 9

| | | ES-MS | tᵣₑₜ |
|---|---|---|---|
| Example | Compound name | (M + H)⁺ | [min] |
| 45 | 2-[4-(2-Methoxy-8-pyridin-3-ylethynyl- | 420 | 2.22 |
| | imidazo[4,5-c]quinolin-1-yl)-phenyl]- | | Grad 2 |
| | ethylamine | | |
| 46 | 2-[4-(2-Cyclopropyl-8-pyridin-3- | 430 | 2.31 |
| | ylethynyl-imidazo[4,5-c]quinolin-1-yl)- | | Grad 2 |
| | phenyl]-ethylamine | | |
| 47 | 2-[4-(2-Isopropyl-8-pyridin-3-ylethynyl- | 432 | 5.25 |
| | imidazo[4,5-c]quinolin-1-yl)-phenyl]- | | Grad 3 |
| | ethylamine | | |

### Example 45a

### {2-[4-(8-Bromo-2-methoxy-imidazo[4,5-c]quinolin-1-yl)-phenyl]-ethyl}-carbamic acid tert-butyl ester

229 mg (0.5 mmol) of {2-[4-(3-amino-6-bromo-quinolin-4-ylamino)phenyl]-ethyl}-carbamic acid tert-butyl ester (Example 1f), 205 mg (1.5 mmol) of tetramethylorthocarbonate and 30 mg (0.5 mmol) of acetic acid are heated for 1 h at 75° C., and then quenched with aqueous sat NaHCO₃ and extracted with EtOAc. The organic layer is washed with brine, dried over MgSO₄, filtered and evaporated in vacuo. The residue is purified by flash chromatography on silica gel (CH₂Cl₂-MeOH 98:2 to 96:4) to provide the title compound as an off-white foam. ES-MS: 497, 499 (M+H)⁺, Br pattern; analytical HPLC: tᵣₑₜ=3.44 minutes (Grad 2).

### Example 46a

### {2-[4-(8-Bromo-2-cyclopropyl-imidazo[4,5-c]quinolin-1-yl)-phenyl]-ethyl}-carbamic acid tert-butyl ester

229 mg (0.5 mmol) of {2-[4-(3-amino-6-bromo-quinolin-4-ylamino)-phenyl]-ethyl}-carbamic acid tert-butyl ester (Example 1f), 88 mg (1.25 mmol) of cyclopropanecarboxaldehyde and 15 mg (0.25 mmol) of acetic acid in 5 ml CH₂Cl₂ are stirred for 44 h at RT, and then quenched with aqueous sat. NaHCO₃ and extracted with CH₂Cl₂. The organic layer is washed with brine, dried over MgSO₄, filtered and evaporated in vacuo. The residue is purified by flash chromatography on silica gel (CH₂Cl₂-MeOH 99:1 to 96:4) to provide the title compound as a yellow foam. ES-MS: 507, 509 (M+H)⁺, Br pattern; analytical HPLC: tᵣₑₜ=3.56 minutes (Grad 2).

### Example 47a

### {2-[4-(8-Bromo-2-isopropyl-imidazo[4,5-c]quinolin-1-yl)-phenyl]-ethyl}-carbamic acid tert-butyl ester

The title compound is obtained as described in Example 46a using isobutyraldehyde (Fluka, Buchs, Switzerland) instead of cyclopropanecarboxaldehyde. ES-MS: 510.9, 512.9 (M+H)⁺, Br pattern; analytical HPLC: tᵣₑₜ=7.51 minutes (Grad 3).

The following compounds (see Table 10) are prepared as described in Example 1 by reacting 6-bromo-4-chloro-3-nitro-quinoline (Example 1c) with the appropriate aniline as in Example 1e.

Example 48 [2-(4-Amino-phenyl)-ethyl]-cyclopropyl-carbamic acid tert-butyl ester (Example 48a);

Example 49 [2-(4-Amino-phenyl)-ethyl]-methyl-carbamic acid tert-butyl ester (Example 49a);

Example 50 [1-(4-Amino-phenyl)-piperidin-4-yl]-carbamic acid tert-butyl ester (Example 50a); and

Example 51 [1-(4-Amino-phenyl)-piperidin-4-ylmethyl]-carbamic acid tert-butyl ester (Example 51a).

TABLE 10

| Exam- | | ES-MS | tᵣₑₜ |
|---|---|---|---|
| ple | Compound name | (M + H)⁺ | [min] |
| 48 | Cyclopropyl-{2-[4-(8-pyridin-3-ylethynyl- | 430 | 2.30 |
| | imidazo[4,5-c]quinolin-1-yl)-phenyl]- | | Grad 2 |
| | ethyl}-amine | | |
| 49 | Methyl-{2-[4-(8-pyridin-3-ylethynyl- | 404 | 2.20 |
| | imidazo[4,5-c]quinolin-1-yl)-phenyl]- | | Grad 2 |
| | ethyl}-amine | | |
| 50 | 1-[4-(8-Pyridin-3-ylethynyl-imidazo[4,5- | 445 | 2.32 |
| | c]quinolin-1-yl)-phenyl]-piperidin- | | Grad 2 |
| | 4-ylamine | | |
| 51 | C-{1-[4-(8-Pyridin-3-ylethynyl- | 459 | 2.35 |
| | imidazo[4,5-c]quinolin-1-yl)-phenyl]- | | Grad 2 |
| | piperidin-4-yl}-methylamine | | |

### Example 48a

### [2-(4-Amino-phenyl)-ethyl]-cyclopropyl-carbamic acid tert-butyl ester

2.13 g (6.91 mmol) of cyclopropyl-[2-(4-nitro-phenyl)-ethyl]-carbamic acid tert-butyl ester (Example 48b) and 220 mg of Pd/C 10% are shacked in 60 ml of MeOH under 1.1 bar of H₂ for 1 h at RT. After completion of the reaction, the catalyst is filtered-off and the filtrate is evaporated in vacuo to give the title compound as an oil. ES-MS: 277 (M+H)⁺; analytical HPLC: tᵣₑₜ=3.25 minutes (Grad 1).

### Example 48b

### Cyclopropyl-[2-(4-nitro-phenyl)-ethyl]-carbamic acid tert-butyl ester

To 1.8 g (8.73 mmol) of cyclopropyl-[2-(4-nitro-phenyl)-ethyl]-amine (Example 48c) and 2.86 g (13.1 mmol) of (Boc)₂O (Fluka, Buchs, Switzerland) in 17 ml of THF are added sat. aqueous NaHCO₃ (15 ml). The reaction mixture is stirred for 2 h at RT, then is extracted with EtOAc(2×). The organic layers are washed with brine, dried over MgSO₄, filtered and evaporated in vacuo. The residue is purified by flash chromatography on silica gel (hexane-EtOAc 8:1 to 7:1) to give the title compound as an oil. ES-MS: 307 (M+H)⁺; analytical HPLC: tᵣₑₜ=5.44 minutes (Grad 1).

### Example 48c

### Cyclopropyl-[2-(4-nitro-phenyl)-ethyl]-amine

2.1 g (9.13 mmol) of 1-(2-bromo-ethyl)-4-nitro-benzene (Fluka, Buchs, CH) and 2.88 g (92.7 mmol) of cyclopropylamine (Fluka, Buchs, Switzerland) in 2 ml of acetonitrile are heated for 2 h at 45° C. and then stirred 17 h at RT. The reaction mixture is quenched with 1 M aqueous K₂CO₃ and extracted with diethylether. The organic layer is dried over MgSO₄, filtered and evaporated in vacuo to give the title compound as an oil. ES-MS: 207 (M+H)⁺; analytical HPLC: tᵣₑₜ=2.40 minutes (Grad 1).

### Example 49a

### [2-(4-Amino-phenyl)-ethyl]-methyl-carbamic acid tert-butyl ester

The title compound is obtained as described in Example 48a starting with methyl-[2-(4-nitro-phenyl)-ethyl]-carbamic acid tert-butyl ester (Example 49b); ES-MS: 251 (M+H)⁺; analytical HPLC: tᵣₑₜ=2.87 minutes (Grad 1).

### Example 49b

### Methyl-[2-(4-nitro-phenyl)-ethyl]-carbamic acid tert-butyl ester

The title compound is obtained as described in Example 48b starting with methyl-[2-(4-nitro-phenyl)-ethyl]-amine (Example 49c); ES-MS: 281 (M+H)⁺; analytical HPLC: tᵣₑₜ=5.06 minutes (Grad 1).

### Example 49c

### Methyl-[2-(4-(4-nitro-phenyl)-ethyl]-amine

The title compound is obtained as described in Example 48c starting with 8 M methylamine in EtOH (Fluka, Buchs, CH); ES-MS: 181 (M+H)⁺; analytical HPLC: tᵣₑₜ=1.89 minutes (Grad 1).

### Example 50a

### [1-(4-Amino-phenyl)-piperidin-4-yl]-carbamic acid tert-butyl ester

The title compound is obtained as described in Example 48a starting with [1-(4-nitro-phenyl)-piperidin-4-yl]-carbamic acid tert-butyl ester (Example 50b); ES-MS: 292 (M+H)⁺; analytical HPLC: tᵣₑₜ=2.41 minutes (Grad 2).

### Example 50b

### [1-(4-nitro-phenyl)-piperidin-4-yl]-carbamic acid tert-butyl ester

212 mg (1.5 mmol) of 4-fluoro-nitrobenzene (Aldrich, Buchs, Switzerland), 331 mg (1.65 mmol) of piperidin-4-yl-carbamic acid tert-butyl ester (Aldrich, Buchs, Switzerland) and 415 mg (3 mmol) of K₂CO₃ in 1.5 ml of DMSO are stirred 1.5 h at RT. After this time, the reaction mixture is treated with aqueous sat. NaHCO₃ and extracted with EtOAc. The organic layer is washed with aqueous sat NaHCO₃ and with brine, dried over MgSO₄, filtered and evaporated to dryness. The residue is purified by flash chromatography on silica gel (hexane-EtOAc 4:1 to 0:1) to provide the title compound as a yellow solid. ES-MS: 322 (M+H)⁺.

### Example 51a

### [1-(4-Amino-phenyl)-piperidin-4-ylmethyl]-carbamic acid tert-butyl ester

The title compound is obtained as described in Example 48a starting with [1-(4-nitro-phenyl)-piperidin-4-ylmethyl]-carbamic acid tert-butyl ester (Example 51b); ES-MS: 306 (M+H)⁺; analytical HPLC: tᵣₑₜ=2.41 minutes (Grad 2).

### Example 51b

### [1-(4-Nitro-phenyl)-piperidin-4-ylmethyl]-carbamic acid tert-butyl ester

The title compound is obtained as described in Example 50b starting with piperidin-4-ylmethyl-carbamic acid tert-butyl ester (Acros, Morris Plains, USA); ES-MS: 336 (M+H)⁺.

The following compounds (see Table 11) are prepared as described in Example 1 by reacting 6-bromo-4-chloro-3-nitro-quinoline (Example 1c) with the appropriate aniline as in Example 1e.

Example 52 N-(4-amino-phenyl)-N-methyl-acetamide (Aldrich, Buchs, CH)

Example 53 (4-amino-phenyl)-methanesulfonamide (Lancaster, Newgate, UK)

Example 54 4-(2-azetidin-1-yl-ethyl)-phenylamine (Example 54a)

Example 55 4-(2-pyrrolidin-1-yl-ethyl)-phenylamine (Example 55a)

Example 56 (4-amino-3-chloro-phenyl)-acetonitrile (Example 56a)

Example 57 (4-amino-2-chloro-phenyl)-acetonitrile (Example 57a)

Example 58 (4-amino-3-methyl-phenyl)-acetonitrile (Example 58a)

Example 59 (4-amino-2-methyl-phenyl)-acetonitrile (Example 59a)

Example 60 (3-amino-phenyl)-acetonitrile (Example 60a)

TABLE 11

| | | ES-MS | tᵣₑₜ |
|---|---|---|---|
| Example | Compound name | (M + H)⁺ | [min] |
| 52 | 2-[4-(2-Methoxy-8-pyridin-3-ylethynyl-imidazo[4,5- | 418 | 2.45 |
| | c]quinolin-1-yl)-phenyl]-ethylamine | | Grad 2 |
| 53 | N-Methyl-C-[4-(8-pyridin-3-ylethynyl-imidazo[4,5- | 454 | 2.42 |
| | c]quinolin-1-yl)-phenyl]-methanesulfonamide | | Grad 2 |
| 54 | 1-[4-(2-Azetidin-1-yl-ethyl)-phenyl]-8-pyridin-3-ylethynyl- | 430 | 2.27 |
| | 1H-imidazo[4,5-c]quinoline | | Grad 2 |
| 55 | 8-Pyridin-3-ylethynyl-1-[4-(2-pyrrolidin-1-yl-ethyl)-phenyl]- | 444 | 2.29 |
| | 1H-imidazo[4,5-c]quinoline | | Grad 2 |
| 56 | [3-Chloro-4-(8-pyridin-3-ylethynyl-imidazo[4,5-c]quinolin-1- | 420 | 2.71 |
| | yl)-phenyl]-acetonitrile | | Grad 2 |
| 57 | [2-Chloro-4-(8-pyridin-3-ylethynyl-imidazo[4,5-c]quinolin-1- | 420 | 2.69 |
| | yl)-phenyl]-acetonitrile | | Grad 2 |
| 58 | [3-Methyl-4-(8-pyridin-3-ylethynyl-imidazo[4,5-c]quinolin-1- | 400 | 2.61 |
| | yl)-phenyl]-acetonitrile | | Grad 2 |
| 59 | [2-Methyl-4-(8-pyridin-3-ylethynyl-imidazo[4,5-c]quinolin-1- | 400 | 2.64 |
| | yl)-phenyl]-acetonitrile | | Grad 2 |
| 60 | [3-(8-Pyridin-3-ylethynyl-imidazo[4,5-c]quinolin-1-yl)- | 386 | 5.63 |
| | phenyl]-acetonitrile | | Grad 3 |

### Example 54a

### 4-(2-Azetidin-1-yl-ethyl)-phenylamine

The title compound is obtained as described in Example 48b starting with 1-[2-(4-nitro-phenyl)-ethyl]-azetidine (Example 54b); ES-MS: 177 (M+H)⁺.

### Example 54b

### 1-[2-(4-Nitro-phenyl)-ethyl]-azetidine

The title compound is obtained as described in Example 48c starting with azetidine (Fluka, Buchs, CH); ES-MS: 207 (M+H)⁺; analytical HPLC: tᵣₑₜ=2.28 minutes (Grad 2).

### Example 55a

### 4-(2-Pyrrolidin-1-yl-ethyl)-phenylamine

The title compound is obtained as described in Example 48b starting with 1-[2-(4-nitro-phenyl)-ethyl]-pyrrolidine (Example 55b); ES-MS: 191 (M+H)⁺.

### Example 55b

### 1-[2-(4-Nitro-phenyl)-ethyl]-pyrrolidine

The title compound is obtained as described in Example 48c starting with pyrrolidine (Fluka, Buchs, Switz.); ES-MS: 221 (M+H)⁺; analytical HPLC: tᵣₑₜ=2.34 minutes (Grad 1).

### Example 56a

### (4-Amino-3-chloro-phenyl)-acetonitrile

2.86 g (21 mmol) of N-chlorosuccinimide are added to a stirred solution of 2.67 g (20 mmol) of (4-amino-phenyl)-acetonitrile (Aldrich, Buchs, CH) in 30 mL of isopropanol. The solution is refluxed for 1 h and then the solvent is removed in vacuo. The crude product is dissolved in EtOAc and water. The layers are separated and the organic layer is washed with brine, dried over MgSO₄ and concentrated in vacuo. The crude residue is purified by chromatography on silica eluting with dichloromethane to afford the title compound; ES-MS: 167 (M+H)⁺.

### Example 57a

### (4-Amino-2-chloro-phenyl)-acetonitrile

2.55 g (13 mmol) of (2-chloro-4-nitro-phenyl)-acetonitrile (Example 57b) and 1 g of Raney-Ni are shacked in 75 mL of MeOH under 1.1 bar of H₂ for 7 h at RT. After completion of the reaction, the catalyst is filtered-off and the filtrate is evaporated to dryness. The residue is purified by flash chromatography on silica gel (hexane-EtOAc 10:1 to 2:1) to give the title compound as a yellowish solid: ES-MS: 167 (M+H)⁺; analytical HPLC: tᵣₑₜ=2.11 minutes (Grad 1).

### Example 57b

### (2-Chloro-4-nitro-phenyl)-acetonitrile

2.94 g (26 mmol) of ethyl cyanoacetate (Fluka, Buchs, CH) and 1.66 g (26 mmol) of KOH in 8 ml of DMSO are stirred for 1 h, then 3.51 g (20 mmol) of 2-chloro-1-fluoronitro-benzene (Aldrich, Buchs, CH) are added and the reaction mixture is stirred for 7.5 h at RT. A solution of 37% aqueous HCl and 5.6 ml of acetic acid is added and the reaction mixture is heated for 3 h at reflux, then quenched with H₂O and extracted with diethylether (2×). The combined organic layers are washed with brine, dried over MgSO₄, filtered and evaporated to dryness. The residue is purified by flash chromatography on silica gel (hexane-EtOAc 10:1 to 6:1) to give the title compound as a gel solid: ES-MS: 195 (M−H)⁻; analytical HPLC: tᵣₑₜ=4.01 minutes (Grad 1).

### Example 58a

### (4-Amino-3-methyl-phenyl)-acetonitrile

1.13 g (6.4 mmol) of (3-methyl-4-nitro-phenyl)-acetonitrile (Example 58b) and 110 mg of Pd 5% on charcoal are shacked in 30 mL of MeOH under 1.1 bar of H₂ for 30 min. After completion of the reaction, the catalyst is filtered-off and the filtrate is evaporated in vacuo to dryness to provide the title compound as an orange solid. ES-MS: 147 (M+H)⁺; analytical HPLC: t_{R}=1.73 minutes (Grad 2).

### Example 58b

### (3-Methyl-4-nitro-phenyl)-acetonitrile

The title compound is obtained as described in Example 57b starting with 4-fluoro-2-methyl-1-nitro-benzene (Aldrich, Buchs, Switzerland); ES-MS: 175 (M−H)⁻; analytical HPLC: tᵣₑₜ=3.90 minutes (Grad 1).

### Example 59a

### (4-Amino-2-methyl-phenyl)-acetonitrile

The title compound is obtained as described in Example 58a starting with (2-methyl-4-nitro-phenyl)-acetonitrile (Example 59b); ES-MS: 147 (M+H)⁺; analytical HPLC: tᵣₑₜ=1.75 minutes (Grad 2).

### Example 59b

### (2-Methyl-4-nitro-phenyl)-acetonitrile

The title compound is obtained as described in Example 57b starting with 4-fluoro-3-methyl-1-nitro-benzene (Aldrich, Buchs, Switzerland); ES-MS: 175 (M−H)⁻; analytical HPLC: tᵣₑₜ=3.91 minutes (Grad 1).

### Example 60a

### (3-Amino-phenyl)-acetonitrile

The title compound is obtained by hydrogenation of 3-nitrophenylacetonitrile (Aldrich, Buchs, Switzerland) as described in Example 48a; ES-MS: 133 (M+H)⁺.

The following compounds (see Table 12) are prepared as described in Example 1 by reacting 6-bromo-4-chloro-3-nitro-quinoline (Example 1c) with 4-(2-dimethylamino-ethyl)-phenylamine (Example 61a) as in Example 1e, and using the following reagents as in Example 1g.

Example 61 triethylorthoformate (Fluka, Buchs, Switzerland);

Example 62 triethylorthoacetate (Fluka, Buchs, Switzerland) as in Example 32;

Example 63 tetramethylorthocarbonate (Aldrich, Buchs, Switzerland) as in Example 45a; and

Example 64 dichloromethylene dimethylimmonium chloride (Fluka, Buchs, Switzerland) (Example 64a).

TABLE 12

| | | ES-MS | tᵣₑₜ |
|---|---|---|---|
| Example | Compound name | (M + H)⁺ | [min] |
| 61 | Dimethyl-{2-[4-(8-pyridin-3-ylethynyl- | 418 | 2.22 |
| | imidazo[4,5-c]quinolin-1-yl)-phenyl]- | | Grad 2 |
| | ethyl}-amine | | |
| 62 | Dimethyl-{2-[4-(2-methyl-8-pyridin-3- | 432 | 2.26 |
| | ylethynyl-imidazo[4,5-c]quinolin-1-yl)- | | Grad 2 |
| | phenyl]-ethyl}-amine | | |
| 63 | {2-[4-(2-Methoxy-8-pyridin-3- | 448 | 2.29 |
| | ylethynyl-imidazo[4,5-c]quinolin-1-yl)- | | Grad 2 |
| | phenyl]-ethyl}-dimethyl-amine | | |
| 64 | {1-[4-(2-Dimethylamino-ethyl)-phenyl]- | 461 | 5.22 |
| | 8-pyridin-3-ylethynyl-1H-imidazo[4,5- | | Grad 3 |
| | c]quinolin-2-yl}-dimethyl-amine | | |

### Example 61a

### 4-(2-Dimethylamino-ethyl)-phenylamine

The title compound is obtained as described in Example 48b starting with dimethyl-[2-(4-nitro-phenyl)-ethyl]-amine (Example 61b); ES-MS: 179 (M+H)⁺.

### Example 61b

### Dimethyl-[2-(4-nitro-phenyl)-ethyl]-amine

The title compound is obtained as described in Example 48c starting with 5.6 M dimethylamine in EtOH (Fluka, Buchs, Switzerland); ES-MS: 165 (M+H)⁺; analytical HPLC: tᵣₑₜ=1.86 minutes (Grad 1).

### Example 64a

### {8-Bromo-1-[4-(2-dimethylamino-ethyl)-phenyl]-1H-imidazo[4,5-c]quinolin-2-yl}-dimethyl-amine

193 mg (0.5 mmol) of 6-bromo-N-4-[4-(2-dimethylamino-ethyl)-phenyl]-quinoline-3,4-diamine, which was obtained as described in Example 1 reacting 6-bromo-4-chloro-3-nitro-quinoline (Example 1c) with 4-(2-dimethylamino-ethyl)phenylamine (Example 61a) as in Example 1e, are dissolved in 5 ml of NMP and 251 mg (1.5 mmol) of dichloromethylene dimethylimmonium chloride (Fluka, Buchs, Switzerland) are added to the stirred solution. The mixture is stirred for 15 min at RT, and then 50 ml of EtOAc are added. The organic phase is washed with 0.1 N aqueous NaOH and water, dried over MgSO₄, filtered and evaportated to dryness. The residue is purified by medium-pressure liquid chromatography to provide the title compound. ES-MS: 437.5, 439.4 (M+H)⁺, Br pattern; analytical HPLC: tᵣₑₜ=5.25 minutes (Grad 3).

The following compounds (see Table 46) are prepared as described in Example 1 by reacting 6-bromochloro-3-nitro-quinoline (Example 1c) with 4-(4-ethyl-piperazin-1-yl)-phenylamine (Acros, Morris Plains, USA) as in Example 1e, and with a cyclisation reaction as in Example 1g or Example 32.

TABLE 13

| | | ES-MS | tᵣₑₜ |
|---|---|---|---|
| Example | Compound name | (M + H)⁺ | [min] |
| 65 | 1-[4-(4-Methyl-piperazin-1-yl)-phenyl]- | 445 | 2.25 |
| | 8-pyridin-3-ylethynyl-1H-imidazo[4,5- | | Grad 2 |
| | c]quinoline | | |
| 66 | 2-Methyl-1-[4-(4-methyl-piperazin-1- | 459 | 2.28 |
| | yl)-phenyl]-8-pyridin-3-ylethynyl-1H- | | Grad 2 |
| | imidazo[4,5-c]quinoline | | |

### Example 67

### 2-Methyl-1-[4-(4-methyl-piperazin-1-yl)-phenyl]-8-(1-oxy-pyridin-3-ylethynyl)-1H-imidazo[4,5-c]quinoline

The title compound is obtained as in Example 66 using 3-ethynyl-pyridine 1-oxide (Example 67a) instead of 3-ethynyl-pyridine; ES-MS: 475 (M+H)⁺; analytical HPLC: tᵣₑₜ=2.24 minutes (Grad 2).

### Example 67a

### 3-Ethynyl-pyridine 1-oxide

To 400 mg (3.88 mmol) of 3-ethynyl-pyridine (Fluka, Buchs, Switzerland) in 40 ml of CH₂Cl₂ cooled with an ice-bath are added 1.41 g (4.65 mmol) of 57% meta-chloroperbenzoic acid. The reaction is then stirred 1 h at 0° C. and 3 h at RT. The reaction mixture is treated with aqueous sat. Na₂CO₃ and extracted with CH₂Cl₂. The organic layer is washed with aqueous sat. Na₂CO₃ and brine, dried over MgSO₄, filtered and evaporated to dryness. The residue is purified by flash chromatography on silica gel (CH₂Cl₂-MeOH 99:1 to 94:6) to provide the title compound as an off-white solid; analytical HPLC: tᵣₑₜ=1.67 minutes (Grad 2).

The following compounds (see Table 14) are prepared as described in Example 1 by reacting 6-bromo-4-chloro-3-nitro-quinoline (Example 1c) with 4-(4-methyl-piperazin-1-ylmethyl)-phenylamine (Example 68a) as in Example 1e, and with a cyclisation reaction as in Example 1g or Example 64.

TABLE 14

| | | ES-MS | tᵣₑₜ |
|---|---|---|---|
| Example | Compound name | (M + H)⁺ | [min] |
| 68 | 1-[4-(4-Methyl-piperazin-1-ylmethyl)- | 459 | 4.97 |
| | phenyl]-8-pyridin-3-ylethynyl-1H- | | Grad 3 |
| | imidazo[4,5-c]quinoline | | |
| 69 | Dimethyl-{1-[4-(4-methyl-piperazin-1- | 502 | 5.11 |
| | ylmethyl)-phenyl]-8-pyridin-3- | | Grad 3 |
| | ylethynyl-1H-imidazo[4,5- | | |
| | c]quinolin-2-yl}-amine | | |

### Example 68a

### 4-(4-Methyl-piperazin-1-ylmethyl)-phenylamine

The title compound is obtained by hydrogenation of 1-methyl-4-(4-nitro-benzyl)-piperazine (Example 68b) as described in Example 67a; ES-MS: 206 (M+H)⁺.

### Example 68b

### 1-Methyl-4-(4-nitro-benzyl)-piperazine

To a solution of 3 g (13.9 mmol) of 4-nitrobenzyl bromide (Fluka, Buchs, Switzerland) in 10 ml of DMF are added 3.08 ml (27.8 mmol) of N-methylpiperazine and 4.8 g (34.7 mmol) of K₂CO₃, and the mixture is stirred for 4.5 h at 80° C. After this time, 150 ml of EtOAc are added and the solution is washed with water, dried over MgSO₄, filtered and evaporated to dryness to provide the title compound. ES-MS: 236 (M+H)⁺.

The following compounds (see Table 15) are prepared as described in Example 1 by reacting 6-bromochloro-3-nitro-quinoline (Example 1c) with 3-fluoro-4-(4-methyl-piperazin-1-yl)-phenylamine (Example 70a) as in Example 1e and with a cyclisation reaction as in Example 1g, Example 32 or Example 45.

TABLE 15

| Exam- | | ES-MS | tᵣₑₜ |
|---|---|---|---|
| ple | Compound name | (M + H)⁺ | [min] |
| 70 | 1-[3-Fluoro-4-(4-methyl-piperazin-1-yl)- | 463 | 2.35 |
| | phenyl]-8-pyridin-3-ylethynyl- | | Grad 2 |
| | 1H-imidazo[4,5-c]quinoline | | |
| 71 | 1-[3-Fluoro-4-(4-methyl-piperazin- | 477 | 2.36 |
| | 1-yl)-phenyl]-2-methyl-8-pyridin- | | Grad 2 |
| | 3-ylethynyl-1H-imidazo[4,5-c]quinoline | | |
| 72 | 1-[3-Fluoro-4-(4-methyl-piperazin-1-yl)- | 493 | 2.40 |
| | phenyl]-2-methoxy-8-pyridin-3-ylethynyl- | | Grad 2 |
| | 1H-imidazo[4,5-c]quinoline | | |

### Example 70a

### 3-Fluoro-4-(4-methyl-piperazin-1-yl)-phenylamine

The title compound is obtained as described in Example 50a starting with 1-(2-fluoro-4-nitro-phenyl)-4-methyl-piperazine (Example 70b); ES-MS: 210 (M+H)⁺.

### Example 70b

### 1-(2-Fluoro-4-nitro-phenyl)-4-methyl-piperazine

The title compound is obtained as described in Example 50b starting with 3,4-difluoro-nitrobenzene (Fluka, Buchs, Switzerland) and N-methylpiperazine (Fluka, Buchs, Switzerland); ES-MS: 240 (M+H)⁺; analytical HPLC: tᵣₑₜ=2.47 minutes (Grad 2).

The following compounds (see Table 16) are prepared as described in Example 1 by reacting 6-bromo-4-chloro-3-nitro-quinoline (Example 1c) with the required aniline.

Example 73 4-(4-amino-phenyl)-piperazine-1-carboxylic acid tert-butyl ester (Example 73a); and

Example 74 4-(4-amino-2-fluoro-phenyl)-piperazine-1-carboxylic acid tert-butyl ester (Example 74a).

TABLE 16

| | | ES-MS | tᵣₑₜ |
|---|---|---|---|
| Example | Compound name | (M + H)⁺ | [min] |
| 73 | 2-Methyl-1-(4-piperazin-1-yl-phenyl)-8- | 445 | 2.30 |
| | pyridin-3-ylethynyl-1H-imidazo[4,5- | | Grad 2 |
| | c]quinoline | | |
| 74 | 1-(3-Fluoro-4-piperazin-1-yl-phenyl)-2- | 463 | 2.34 |
| | methyl-8-pyridin-3-ylethynyl-1H- | | Grad 2 |
| | imidazo[4,5-c]quinoline | | |

### Example 73a

### 4-(4-Amino-phenyl)-piperazine-1-carboxylic acid tert-butyl ester

The title compound is obtained as described in Example 50a starting with 4-(4-nitro-phenyl)-piperazine-1-carboxylic acid tert-butyl ester (Example 73b); ES-MS: 278 (M+H)⁺; analytical HPLC: tᵣₑₜ=2.71 minutes (Grad 2).

### Example 73b

### 4-(4-Nitro-phenyl)-piperazine-1-carboxylic acid tert-butyl ester

The title compound is obtained as described in Example 50b starting with piperazine-1-carboxylic acid tert-butyl ester (Fluka, Buchs, CH); ES-MS: 308 (M+H)⁺.

### Example 74a

### 4-(4-Amino-2-fluoro-phenyl)-piperazine-1-carboxylic acid tert-butyl ester

The title compound is obtained as described in Example 50a starting with 4-(2-fluoro-4-nitro-phenyl)-piperazine-1-carboxylic acid tert-butyl ester (Example 74b); ES-MS: 296 (M+H)⁺; analytical HPLC: tᵣₑₜ=2.87 minutes (Grad 2).

### Example 74b

### 4-(2-Fluoro-4-nitro-phenyl)-piperazine-1-carboxylic acid tert-butyl ester

The title compound is obtained as described in Example 50b starting with 3,4-difluoro-nitrobenzene (Fluka, Buchs, Switzerland); ES-MS: 326 (M+H)⁺.

### Example 75

### 1-[4-(4-Ethyl-piperazin-1-yl)-3-fluoro-phenyl]-2-methyl-8-pyridin-3-ylethynyl-1H-imidazo[4,5-c]quinoline

80 mg (0.173 mmol) of 1-(3-fluoro-4-piperazin-1-yl-phenyl)-2-methyl-8-pyridin-3-ylethynyl-1H-imidazo[4,5-c]quinoline (Example 74), 27 mg (0.173 mmol) of iodoethane and 34 mg (0.259 mmol) of ethyl-diisopropyl-amine in 2 ml CH₂Cl₂-MeOH (5:1) are stirred 5 days at RT and then 8 mg (0.052 mmol) of iodoethane are added and the reaction mixture stirred for 2 days at RT. The reaction mixture is quenched with aqueous sat. NaHCO₃ and extracted with EtOAc. The organic layer is washed with aqueous sat. NaHCO₃, dried over MgSO₄, filtered and evaporated to dryness. The residue is purified by prep. HPLC. The pure fractions are concentrated, basified with NaHCO₃ and extracted with EtOAc (3×). The organic layers are dried over MgSO₄, filtered and evaporated to dryness to give the title compound. ES-MS: 491 (M+H)⁺; analytical HPLC: tᵣₑₜ=2.42 minutes (Grad 2).

The following compounds (see Table 50) are prepared as described in Example 1 by reacting 6-bromo-4-chloro-3-nitro-quinoline (Example 1c) with 5-amino-2-(4-methyl-piperazin-1-yl)-benzonitrile (Example 76a) as in Example 1e, and with a cyclisation reaction as in Example 1g, Example 32, Example 45 or Example 64.

TABLE 17

| Exam- | | ES-MS | tᵣₑₜ |
|---|---|---|---|
| ple | Compound name | (M + H)⁺ | [min] |
| 76 | 2-(4-Methyl-piperazin-1-yl)-5-(8- | 470 | 2.33 |
| | pyridin-3-ylethynyl-imidazo[4,5- | | Grad 2 |
| | c]quinolin-1-yl)-benzonitrile | | |
| 77 | 2-(4-Methyl-piperazin-1-yl)-5-(2-methyl- | 484 | 2.32 |
| | 8-pyridin-3-ylethynyl-imidazo[4,5- | | Grad 2 |
| | c]quinolin-1-yl)-benzonitrile | | |
| 78 | 5-(2-Methoxy-8-pyridin-3-ylethynyl- | 500 | 2.35 |
| | imidazo[4,5-c]quinolin-1-yl)-2-(4- | | Grad 2 |
| | methyl-piperazin-1-yl)-benzonitrile | | |
| 79 | 5-(2-Dimethylamino-8-pyridin-3- | 513 | 2.39 |
| | ylethynyl-imidazo[4,5-c]quinolin-1- | | Grad 2 |
| | yl)-2-(4-methyl-piperazin-1-yl)- | | |
| | benzonitrile | | |

### Example 76a

### 5-Amino-2-(4-methyl-piperazin-1-yl)-benzonitrile

The title compound is obtained as described in Example 50a starting with 2-(4-methyl-piperazin-1-yl)-5-nitro-benzonitrile (Example 76b); ES-MS: 217 (M+H)⁺.

### Example 76b

### 2-(4-Methyl-piperazin-1-yl)-5-nitro-benzonitrile

1 g (6.02 mmol) of 2-fluoro-5-nitro-benzonitrile (Aldrich, Buchs, Switzerland), 663 mg (6.62 mmol) of N-methylpyperazine (Fluka, Buchs, CH) and 2.5 g (18.1 mmol) of K₂CO₃ in 12 ml DMF are stirred for 30 min at rt, then the reaction mixture is evaporated to dryness. The residue is treated with water and extracted with EtOAc (2×). The combined organic layers are washed with aqueous sat. NaHCO₃ (3×), dried over MgSO₄, filtered and evaporated to dryness. The residue is purified by flash chromatography on silica gel (CH₂Cl₂-MeOH—NEt₃ 196:4:1 to 193:7:1) to provide the title compound as a yellow solid: ES-MS: 247 (M+H)⁺; analytical HPLC: tᵣₑₜ=2.38 minutes (Grad 2).

The following compounds (see Table 18) are prepared as described in Example 1 by reacting 6-bromo-4-chloro-3-nitro-quinoline (Example 1c) with 4-(4-amino-2-cyano-phenyl)-piperazine-1-carboxylic acid tert-butyl ester (Example 80a) as in Example 1e, and with a cyclisation reaction as in Example 1g, Example 32, Example 45 or Example 64.

TABLE 18

| | | ES-MS | tᵣₑₜ |
|---|---|---|---|
| Example | Compound name | (M + H)⁺ | [min] |
| 80 | 2-Piperazin-1-yl-5-(8-pyridin-3- | 456 | 2.30 |
| | ylethynyl-imidazo[4,5-c]quinolin- | | Grad 2 |
| | 1-yl)-benzonitrile | | |
| 81 | 5-(2-Methyl-8-pyridin-3-ylethynyl- | 470 | 2.29 |
| | imidazo[4,5-c]quinolin-1-yl)- | | Grad 2 |
| | 2-piperazin-1-yl-benzonitrile | | |
| 82 | 5-(2-Methoxy-8-pyridin-3- | 486 | 2.32 |
| | ylethynyl-imidazo[4,5-c]quinolin- | | Grad 2 |
| | 1-yl)-2-piperazin-1-yl-benzonitrile | | |
| 83 | 5-(2-Dimethylamino-8-pyridin-3- | 499 | 2.36 |
| | ylethynyl-imidazo[4,5-c]quinolin- | | Grad 2 |
| | 1-yl)-2-piperazin-1-yl-benzonitrile | | |

### Example 80a

### 4-(4-Amino-2-cyano-phenyl)-piperazine-1-carboxylic acid tert-butyl ester

The title compound is obtained as described in Example 50a starting with 4-(2-cyano-4-nitro-phenyl)-piperazine-1-carboxylic acid tert-butyl ester (Example 80b); ES-MS: 303 (M+H)⁺; analytical HPLC: tᵣₑₜ=2.99 minutes (Grad 2).

### Example 80b

### 4-(2-Cyano-4-nitro-phenyl)-piperazine-1-carboxylic acid tert-butyl ester SH-242

1 g (6.02 mmol) of 2-fluoro-5-nitro-benzonitrile (Aldrich, Buchs, Switzerland), 1.23 g (6.62 mmol) of piperazine-1-carboxylic acid tert-butyl este (Fluka, Buchs, Switzerland) and 1.17 g (9.3 mmol) of ethyldiisopropylamine in 5 ml DMSO are stirred for 30 min at RT, then the reaction mixture is treated with water and extracted with EtOAc (2×). The combined organic layers are washed with brine, dried over MgSO₄, filtered and evaporated to dryness to give the title compound as a yellow solid: analytical HPLC: tᵣₑₜ=4.06 minutes (Grad 2).

The following compounds (see Table 19) are prepared as described in Example 1 by reacting 6-bromo-chloro-3-nitro-quinoline (Example 1c) with 3-amino-benzonitrile (Fluka, Buchs, Switzerland) s in Example 1e, and with a cyclisation reaction as in Example 1g, Example 32, Example 45 or Example 64.

TABLE 19

| | | ES-MS | tᵣₑₜ |
|---|---|---|---|
| Example | Compound name | (M + H)⁺ | [min] |
| 84 | 3-(8-Pyridin-3-ylethynyl-imidazo[4,5- | 372 | 5.68 |
| | c]quinolin-1-yl)-benzonitrile | | Grad 3 |
| 85 | 3-(2-Methyl-8-pyridin-3-ylethynyl- | 386 | 5.68 |
| | imidazo[4,5-c]quinolin-1-yl)- | | Grad 3 |
| | benzonitrile | | |
| 86 | 3-(2-Methoxy-8-pyridin-3-ylethynyl- | 402 | 5.76 |
| | imidazo[4,5-c]quinolin-1-yl)- | | Grad 3 |
| | benzonitrile | | |
| 87 | 3-(2-Dimethylamino-8-pyridin-3- | 415 | 5.78 |
| | ylethynyl-imidazo[4,5-c]quinolin-1- | | Grad 3 |
| | yl)-benzonitrile | | |

### Example 88

### 3-(8-Pyridin-3-ylethynyl-imidazo[4,5-c]quinolin-1-yl)-benzylamine

The title compound is obtained as described in Example 52 starting with 3-(8-bromo-imidazo[4,5-c]quinolin-1-yl)-benzylamine (Example 88a); ES-MS: 376 (M+H)⁺; analytical HPLC: tᵣₑₜ=2.17 minutes (Grad 2).

### Example 88a

### 3-(8-Bromo-imidazo[4,5-c]quinolin-1-yl)-benzylamine

240 mg (0.687 mmol) of 3-(8-bromo-imidazo[4,5-c]quinolin-1-yl)-benzonitrile (intermediate in Example 88; ES-MS: 350 (M+H)⁺) and 0.1 g of Raney-Ni are shacked in 6 mL of THF-[MeOH/NH₃ (5%)] (1:1) under 1.1 bar of H₂ for 10 h at 42° C. After completion of the reaction, the catalyst is filtered-off and the filtrate is evaporated in vacuo to give the title compound as an off-white solid: ES-MS: 353, 355 (M+H)⁺, Br pattern; analytical HPLC: tᵣₑₜ=2.19 minutes (Grad 2).

The following compounds (see Table 20) are prepared as described in Example 1 by reacting 6-bromo-4-chloro-3-nitro-quinoline (Example 1c) with 4-amino-benzonitrile (Fluka, Buchs, Switzerland) as In Example 1e, and with a cyclisation reaction as in Example 1g, Example 32, Example 45 or Example 64.

TABLE 20

| | | ES-MS | tᵣₑₜ |
|---|---|---|---|
| Example | Compound name | (M + H)⁺ | [min] |
| 89 | 4-(8-Pyridin-3-ylethynyl-imidazo[4,5- | 372 | 5.72 |
| | c]quinolin-1-yl)-benzonitrile | | Grad 3 |
| 90 | 4-(2-Methyl-8-pyridin-3-ylethynyl- | 386 | 5.71 |
| | imidazo[4,5-c]quinolin-1-yl)- | | Grad 3 |
| | benzonitrile | | |
| 91 | 4-(2-Methoxy-8-pyridin-3-ylethynyl- | 402 | 5.83 |
| | imidazo[4,5-c]quinolin-1-yl)- | | Grad 3 |
| | benzonitrile | | |
| 92 | 4-(2-Dimethylamino-8-pyridin- | 415 | 5.83 |
| | 3-ylethynyl-imidazo[4,5-c]quinolin-1- | | Grad 3 |
| | yl)-benzonitrile | | |

The following compounds (see Table X14) are prepared as described in Example 1 by reacting 6-bromo-4-chloro-3-nitro-quinoline (Example 1c) with (4-amino-phenyl)-acetonitrile (Aldrich, Buchs, Switzerland) as in Example 1e, and with a cyclisation reaction as in Example 1g, Example 32, Example 36, Example 45, Example 64 or Example 98a.

TABLE 21

| | | ES-MS | tᵣₑₜ |
|---|---|---|---|
| Example | Compound name | (M + H)⁺ | [min] |
| 93 | [4-(8-Pyridin-3-ylethynyl-imidazo[4,5- | 386 | 2.52 |
| | c]quinolin-1-yl)-phenyl]-acetonitrile | | Grad 2 |
| 94 | [4-(2-Methyl-8-pyridin-3-ylethynyl- | 400 | 2.54 |
| | imidazo[4,5-c]quinolin-1-yl)- | | Grad 2 |
| | phenyl]-acetonitrile | | |
| 95 | [4-(2-Ethyl-8-pyridin-3-ylethynyl- | 414 | 2.71 |
| | imidazo[4,5-c]quinolin-1- | | Grad 2 |
| | yl)-phenyl]-acetonitrile | | |
| 96 | [4-(2-Methoxy-8-pyridin-3-ylethynyl- | 416 | 2.63 |
| | imidazo[4,5-c]quinolin-1-yl)- | | Grad 2 |
| | phenyl]-acetonitrile | | |
| 97 | [4-(2-Dimethylamino-8-pyridin-3- | 429 | 5.78 |
| | ylethynyl-imidazo[4,5-c]quinolin-1- | | Grad 3 |
| | yl)-phenyl]-acetonitrile | | |
| 98 | {4-[2-(3-Dimethylamino-propyl)- | 471 | 5.28 |
| | 8-pyridin-3-ylethynyl-imidazo[4,5- | | Grad 3 |
| | c]quinolin-1-yl]-phenyl}-acetonitrile | | |

### Example 98a

### {4-[8-Bromo-2-(3-dimethylamino-propyl)-imidazo[4,5-c]quinolin-1-yl]-phenyl}-acetonitrile

33 mg (0.078 mmol) of {4-[8-bromo-2-(3-hydroxy-propyl)-imidazo[4,5-c]quinolin-1-yl]-phenyl}-acetonitrile (Example 98b) are dissolved in 3 ml of anydrous pyridine and the solution is cooled to −18° C. To this solution, 69 mg (0.35 mmol) of p-toluenesulfonyl chloride are added and the mixture is stirred for 3 days at −18° C. After this time, 50 ml of EtOAc are added and the solution is extracted with water. The organic phase is evaporated to dryness and the residue is dissolved in 2 ml of ethanol. To this solution, 0.28 ml (0.16 mmol) of dimethylamine are added and the mixture is refluxed for 1 h. After this time, the mixture is evaporated to dryness and the residue is purified by medium-pressure liquid chromatography to provide the title compound; ES-MS: 448, 450 (M+H)⁺, Br pattern; analytical HPLC: tᵣₑₜ=5.62 minutes (Grad 3).

### Example 98b

### {4-[8-Bromo-2-(3-hydroxy-propyl)-imidazo[4,5-c]quinolin-1-yl]-phenyl}-acetonitrile

0.23 ml (0.23 mmol) of borane tetrahydrofuran complex solution are added to a solution of 90 mg (0.21 mmol) of 3-[8-bromo-1-(4-cyanomethyl-phenyl)-1H-imidazo[4,5-c]quinolin-2-yl]-propionic acid (Example 98c) in 5 ml of THF. The mixture is stirred for 4 h at room temperature. After this time, the reaction is quenched with 95% TFA and the pH is then adjusted to 9-10 by addition of 2 N NaOH. The mixture is extracted with EtOAc and the organic phase is washed with water, dried over MgSO₄, filtered and evaporated to dryness to provide the title compound. ES-MS: 421, 423 (M+H)⁺, Br pattern; analytical HPLC: tᵣₑₜ=5.95 minutes (Grad 3).

### Example 98c

### 3-[8-Bromo-1-(4-cyanomethyl-phenyl)-1H-imidazo[4,5-c]quinolin-2-yl]-propionic acid

The title compound is obtained as described in Example 46a using [4-(3-amino-6-bromo-quinolin-4-ylamino)-phenyl]-acetonitrile (intermediate in Example 93; ES-MS: 353, 355 (M+H)⁺, Br pattern) and succinaldehydic acid (Fluka, Buchs, Switzerland); ES-MS: 436.8 (M+H)⁺; analytical HPLC: tᵣₑₜ=5.98 minutes (Grad 3).

The following compounds (see Table 22) are prepared as described in Example 92 by using the required alkyne as in Example 42 or Example 67.

TABLE 22

| | | ES-MS | tᵣₑₜ |
|---|---|---|---|
| Example | Compound name | (M + H)⁺ | [min] |
| 99 | {4-[8-(6-Morpholin-4-yl-pyridin-3- | 471 | 2.64 |
| | ylethynyl)-imidazo[4,5-c]quinolin-1- | | Grad 2 |
| | yl]-phenyl}-acetonitrile | | |
| 100 | {4-[8-(1-Oxy-pyridin-3-ylethynyl)- | 402 | 2.49 |
| | imidazo[4,5-c]quinolin-1-yl]- | | Grad 2 |
| | phenyl}-acetonitrile | | |

The following compounds (see Table X16) are prepared as in Example 93 using [4-(4-amino-bromo-imidazo[4,5-c]quinolin-1-yl)-phenyl]-acetonitrile (Example 101a) or [4-(8-bromo-4-methylamino-imidazo[4,5-c]quinolin-1-yl)-phenyl]-acetonitrile (Example 102a).

TABLE 23

| | | ES-MS | tᵣₑₜ |
|---|---|---|---|
| Example | Compound name | (M + H)⁺ | [min] |
| 101 | [4-(4-Amino-8-pyridin-3-ylethynyl- | 401 | 2.60 |
| | imidazo[4,5-c]quinolin-1-yl)- | | Grad 2 |
| | phenyl]-acetonitrile | | |
| 102 | [4-(4-Methylamino-8-pyridin-3- | 415 | 2.66 |
| | ylethynyl-imidazo[4,5-c]quinolin- | | Grad 2 |
| | 1-yl)-phenyl]-acetonitrile | | |

### Example 101a

### [4-(4-Amino-8-bromo-imidazo[4,5-c]quinolin-1-yl)-phenyl]-acetonitrile

172 mg (0.433 mmol) of [4-(8-bromo-4-chloro-imidazo[4,5-c]quinolin-1-yl)-phenyl]-acetonitrile (Example 101b) and 3 ml (6 mmol) of 2 M NH₃ in MeOH are heated in a microwave oven for 10 h at 130° C., then the reaction mixture is evaporated to dryness. The residue is purified by flash chromatography on silica gel (CH₂Cl₂-MeOH 1:0 to 96:4) to provide the title compound as a brownish solid: ES-MS: 378, 380 (M+H)⁺, Br pattern; analytical HPLC: tᵣₑₜ=2.96 minutes (Grad 2).

### Example 101b

### [4-(8-Bromo-4-chloro-imidazo[4,5-c]quinolin-1-yl)-phenyl]-acetonitrile

300 mg (0.791 mmol) of [4-(8-bromo-5-oxy-imidazo[4,5-c]quinolin-1-yl)-phenyl]-acetonitrile (Example 101c) and 364 mg (2.37 mmol) of POCl₃ in 8 ml toluene-DMF (39:1) are heated for 4 h at 70° C. The reaction mixture is quenched with aqueous sat. NaHCO₃ and extracted with EtOAc (2×). The organic layers are washed with aqueous sat. NaHCO₃ and brine, dried over MgSO₄, filtered and evaporated in vacuo to provide the title compound as a brownish solid: ES-MS: 397, 399 (M+H)⁺, Br pattern; analytical HPLC: tᵣₑₜ=3.81 minutes (Grad 2).

### Example 101c

### [4-(8-Bromo-5-oxy-imidazo[4,5-c]quinolin-1-yl)-phenyl]-acetonitrile

340 mg (0.936 mmol) of [4-(8-bromo-imidazo[4,5c]quinolin-1-yl)-phenyl]-acetonitrile (intermediate in Example 93; ES-MS: 363, 365 M(+H)⁺), 119 mg (1.12 mmol) of Na₂CO3 and 312 mg (1.03 mmol) of 57% meta-chloroperbenzoic acid in 10 ml of chloroform are stirred for 20 h at RT. The reaction mixture is quenched with aqueous sat. Na₂CO₃ and extracted with CH₂Cl₂ (2×). The organic layers are washed with brine, dried over MgSO₄, filtered and evaporated in vacuo to give the title compound as an orange solid: ES-MS: 379, 381 (M+H)⁺, Br pattern; analytical HPLC: tᵣₑₜ=3.06 minutes (Grad 2).

### Example 102a

### [4-(8-Bromo-4-methylamino-imidazo[4,5-c]quinolin-1-yl)-phenyl]-acetonitrile

The title compound is obtained as described in Example 101a using 8 M methylamine in EtOH for 2 h at 120° C.; ES-MS: 392, 394 (M+H)⁺, Br pattern; analytical HPLC: tᵣₑₜ=3.00 minutes (Grad 2).

The following compounds (see Table 24) are prepared as described in Example 1 by reacting 6-bromo-4-chloro-3-nitro-quinoline (Example 1c) with (4-amino-2-fluoro-phenyl)-acetonitrile (Example 103a) as in Example 1e, and with a cyclisation reaction as in Example 19, Example 32, Example 45 or Example 64.

TABLE 24

| | | ES-MS | tᵣₑₜ |
|---|---|---|---|
| Example | Compound name | (M + H)⁺ | [min] |
| 103 | [2-Fluoro-4-(8-pyridin-3-ylethynyl- | 404 | 2.61 |
| | imidazo[4,5-c]quinolin-1-yl)- | | Grad 2 |
| | phenyl]-acetonitrile | | |
| 104 | [2-Fluoro-4-(2-methyl-8-pyridin-3- | 418 | 2.61 |
| | ylethynyl-imidazo[4,5-c]quinolin- | | Grad 2 |
| | 1-yl)-phenyl]-acetonitrile | | |
| 105 | [2-Fluoro-4-(2-methoxy-8-pyridin-3- | 434 | 2.69 |
| | ylethynyl-imidazo[4,5-c]quinolin- | | Grad 2 |
| | 1-yl)-phenyl]-acetonitrile | | |
| 106 | [4-(2-Dimethylamino-8-pyridin-3- | 447 | 2.69 |
| | ylethynyl-imidazo[4,5-c]quinolin-1- | | Grad 2 |
| | yl)-2-fluoro-phenyl]-acetonitrile | | |

### Example 103a

### (4-Amino-2-fluoro-phenyl)-acetonitrile

1.55 g (8.6 mmol) of (2-fluoro-4-nitro-phenyl)-acetonitrile (Example 103b) and 160 mg of Pd 5% on charcoal are shacked in 45 mL of MeOH under 1.1 bar of H₂ for 4 h. After completion of the reaction, the catalyst is filtered-off and the filtrate is evaporated in vacuo to dryness to provide the title compound as a brown solid: analytical HPLC: t_{R}=1.76 minutes (Grad 2).

### Example 103b

### (2-Fluoro-4-nitro-phenyl)-acetonitrile

1.59 g (10 mmol) of 3,4-difluoro-1-nitrobenzene, 1.9 g (13.8 mmol) of finely-powdered K₂CO₃, 16.6 mg (0.1 mmol) of KI and 1.24 g (11 mmol) of ethyl cyanoacetate in 10 mL DMF are stirred for 4 h at RT, and then 1 h at 50° C. and 1 h at 100° C. The reaction mixture is quenched with aqueous 1 M citric acid and extracted with EtOAc. The combined organic layers are washed with brine, dried over MgSO₄, filtered and evaporated in vacuo. The residue is treated with 1 mL of HCl (37%) in 10 mL of H₂O-acetic acid (3:1) for 8 h at 100° C. After this time, the reaction mixture is quenched with saturated aqueous NaHCO₃ and extracted with ether. The combined organic layers are washed with aqueous NaHCO₃, brine and dried over MgSO₄. The organic phase is evaporated in vacuo to dryness to give the title compound as a pale yellow solid: ES-MS: 179 (M−H)⁻, analytical HPLC: t_{R}=3.69 minutes (Grad 2);

The following compounds (see Table 25) are prepared as described in Example 1 by reacting 6-bromo-4-chloro-3-nitro-quinoline (Example 1c) with 2-(4-amino-phenyl)-2-methyl-propionitrile (Example 107a) as in Example 1e, and with a cyclisation reaction as in Example 1g, Example 32 or Example 45.

TABLE 25

| | | ES-MS | tᵣₑₜ |
|---|---|---|---|
| Example | Compound name | (M + H)⁺ | [min] |
| 107 | 2-Methyl-2-[4-(8-pyridin-3-ylethynyl- | 414 | 2.86 |
| | imidazo[4,5-c]quinolin-1-yl)- | | Grad 2 |
| | phenyl]-propionitrile | | |
| 108 | 2-Methyl-2-[4-(2-methyl-8-pyridin-3- | 428 | 2.85 |
| | ylethynyl-imidazo[4,5-c]quinolin- | | Grad 2 |
| | 1-yl)-phenyl]-propionitrile | | |
| 109 | 2-[4-(2-Methoxy-8-pyridin-3- | 444 | 2.92 |
| | ylethynyl-imidazo[4,5-c]quinolin- | | Grad 2 |
| | 1-yl)-phenyl]-2-methyl-propionitrile | | |

### Example 107a

### (2-(4-Amino-phenyl)-2-methyl-proplonitrile

3.8 g (20 mmol) of 2-methyl-2-(4-nitro-phenyl)-propionitrile (Example 10b) and 1 g of Raney-Ni are shacked in 50 mL of THF-MeOH (1:1) under 1.1 bar of H₂ for 4 h at RT. After completion of the reaction, the catalyst is filtered-off and the filtrate is evaporated to dryness. The residue is purified by flash chromatography on silica gel (hexane-EtOAc 3:1 to 1:2) to give the title compound as an oil: ES-MS: 161 (M+H)⁺; analytical HPLC: tᵣₑₜ=2.13 minutes (Grad 2).

### Example 107b

### 2-Methyl-2-(4-nitro-phenyl)-propionitrile

To 4.5 g (27.8 mmol) of (4-nitro-phenyl)-acetonitrile (Fluka, Buchs, Switzerland), 500 mg (1.55 mmol) of tetrabutylammonium bromide (Fluka, Buchs, Switzerland) and 13 g (91.6 mmol) of iodomethane in 37.5 mL of CH₂Cl₂ are added 3 g (75 mmol) of NaOH in 37.5 ml of water. The reaction mixture is stirred for 12 h at RT, then the organic layer is separated and dried over MgSO₄, and evaporated to dryness. The residue is dissolved in diethylether and treated with black charcoal for 30 min, filtered over Celite and evaporated in vacuo to give the title compound as a pale yellow solid: analytical HPLC: tᵣₑₜ=3.60 minutes (Grad 2).

The following compounds (see Table 26) are prepared as described in Example 1 by reacting 6-bromo-4-chloro-3-nitro-quinoline (Example 1c) with 2-(4-amino-2-fluoro-phenyl)-2-methyl-propionitrile (Example 109a) as in Example 1e, and with a cyclisation reaction as in Example 1g, Example 32 or Example 64.

TABLE 26

| | | ES-MS | tᵣₑₜ |
|---|---|---|---|
| Example | Compound name | (M + H)⁺ | [min] |
| 110 | 2-[2-Fluoro-4-(8-pyridin-3-ylethynyl- | 432 | 2.86 |
| | imidazo[4,5-c]quinolin-1-yl)- | | Grad 2 |
| | phenyl]-2-methyl-propionitrile | | |
| 111 | 2-[2-Fluoro-4-(2-methyl-8-pyridin-3- | 446 | 2.88 |
| | ylethynyl-imidazo[4,5-c]quinolin-1-yl)- | | Grad 2 |
| | phenyl]-2-methyl-propionitrile | | |
| 112 | 2-[4-(2-Dimethylamino-8-pyridin- | 475 | 2.95 |
| | 3-ylethynyl-imidazo[4,5-c]quinolin-1- | | Grad 2 |
| | yl)-2-fluoro-phenyl]-2-methyl- | | |
| | propionitrile | | |

### Example 10a

### 2-(4-Amino-2-fluoro-phenyl)-2-methyl-proplonitrile

The title compound is obtained as described in Example 48a starting with 2-(2-fluoro-4-nitro-phenyl)-2-methyl-propionitrile (Example 110b); ES-MS: 251 (M+H)⁺; analytical HPLC: tᵣₑₜ=2.87 minutes (Grad 2).

### Example 110b

### 2-(2-Fluoro-4-nitro-phenyl)-2-methyl-propionitrile

The title compound is obtained as described in Example 107b starting with (2-fluoro-4-nitro-phenyl)-acetonitrile (Example 103a); analytical HPLC: tᵣₑₜ=3.64 minutes (Grad 2).

The following compounds (see Table 27) are prepared as described in Example 1 by reacting 6-bromo-4-chloro-3-nitro-quinoline (Example 1c) with 3-(4-amino-phenyl)-propionitrile (Example 113a) as in Example 1e, and with a cyclisation reaction as in Example 1g, Example 32, Example 45 or Example 64.

TABLE 27

| | | ES-MS | tᵣₑₜ |
|---|---|---|---|
| Example | Compound name | (M + H)⁺ | [min] |
| 113 | 3-[4-(8-Pyridin-3-ylethynyl- | 400 | 2.57 |
| | imidazo[4,5-c]quinolin-1-yl)- | | Grad 2 |
| | phenyl]-propionitrile | | |
| 114 | 3-[4-(2-Methyl-8-pyridin-3-ylethynyl- | 414 | 2.63 |
| | imidazo[4,5-c]quinolin-1-yl)-phenyl]- | | Grad 2 |
| | propionitrileBAE852/SH-137 | | |
| 115 | 3-[4-(2-Methoxy-8-pyridin-3- | 430 | 2.71 |
| | ylethynyl-imidazo[4,5-c]quinolin-1-yl)- | | Grad 2 |
| | phenyl]-propionitrile | | |
| 116 | 3-[4-(2-Dimethylamino-8-pyridin-3- | 443 | 5.88 |
| | ylethynyl-imidazo[4,5-c]quinolin-1-yl)- | | Grad 3 |
| | phenyl]-propionitrile | | |

### Example 113a

### 3-(4-Amino-phenyl)-propionitrile

0.78 g (4.4 mmol) of 3-(4-nitro-phenyl)-propionitrile (Example 113b) are dissolved in 40 mL of MeOH:THF (1:1) and hydrogenated at RT in the presence of 50 mg of Pd—C 10%. After completion of the reaction, the catalyst is filtered-off and washed with methanol. The organic solvent is evaporated to dryness to provide the title compound; ES-MS: 147.3 (M+H)⁺.

### Example 113b

### 3-(4-Nitro-phenyl)-propionitrile

3.45 of (15 mmol) of 4-nitrophenethyl bromide are dissolved in 50 mL of ethanol and 0.81 g (16.5 mmol) of sodium cyamide are added. The solution is stirred for 4 h at RT and then evaporated to dryness. The crude compound is dissolved in 100 mL of EtOAc, and the organic solution is extracted with water, brine, dried over MgSO₄ and evaporated to dryness. The crude compound is purified by medium-pressure liquid chromatography to provide the title compound; ES-MS: 175.3 (M−H)⁻.

The following compounds (see Table 28) are prepared as described in Example 1 by reacting 6-bromo-4-chloro-3-nitro-quinoline (Example 1c) with 1-(4-amino-2-fluoro-phenyl)-pyrrolidin-2-one (Example 117a) as in Example 1e, and with a cyclisation reaction as in Example 1g, Example 32, Example 45 or Example 64.

TABLE 28

| | | ES-MS | tᵣₑₜ |
|---|---|---|---|
| Example | Compound name | (M + H)⁺ | [min] |
| 117 | 1-[2-Fluoro-4-(8-pyridin-3-ylethynyl- | 448 | 2.56 |
| | imidazo[4,5-c]quinolin-1-yl)- | | Grad 2 |
| | phenyl]-pyrrolidin-2-one | | |
| 118 | 1-[2-Fluoro-4-(2-methyl-8-pyridin-3- | 462 | 2.58 |
| | ylethynyl-imidazo[4,5-c]quinolin-1-yl)- | | Grad 2 |
| | phenyl]-pyrrolidin-2-one | | |
| 119 | 1-[2-Fluoro-4-(2-methoxy-8-pyridin-3- | 478 | 2.66 |
| | ylethynyl-imidazo[4,5-c]quinolin-1-yl)- | | Grad 2 |
| | phenyl]-pyrrolidin-2-one | | |
| 120 | 1-[4-(2-Dimethylamino-8-pyridin-3- | 491 | 2.67 |
| | ylethynyl-imidazo[4,5-c]quinolin-1-yl)- | | Grad 2 |
| | 2-fluoro-phenyl]-pyrrolidin-2-one | | |

### Example 117a

### 1-(4-Amino-2-fluoro-phenyl)-pyrrolidin-2-one

The title compound is obtained as described in Example 48a starting with 1-(2-fluoro-4-nitro-phenyl)-pyrrolidin-2-one (Example 177b); ES-MS: 195 (M+H)⁺; analytical HPLC: tᵣₑₜ=1.91 minutes (Grad 2).

### Example 177b

### 1-(2-Fluoro-4-nitro-phenyl)-pyrrolidin-2-one

To 468 mg (5.5 mmol) of 2-pyrrolidone (Fluka, Buchs, Switzerland) in 10 ml of DMF cooled with an ice-bath are added 240 mg (5.5 mmol) of 55% NaH in oil. The reaction mixture is stirred for 30 min at 0° C. and for 30 min at RT, then are added 795 mg (5 mmol) of 3,4-difluoronitrobenzene (Aldrich, Buchs, Switzerland) and the reaction mixture is stirred for 1 h at RT. The reaction mixture is quenched with 1 M aqueous HCl and extracted with EtOAc (2×). The organic layers are washed with aqueous sat. NaHCO₃ and with brine (3×), dried over MgSO₄, filtered and evaporated. The residue is purified by flash chromatography on silica gel (hexane-EtOAc 5:1 to 1:3) to give the title compound; ES-MS: 225 (M+H)⁺; analytical HPLC: tᵣₑₜ=2.99 minutes (Grad 2).

The following compounds (see Table 29) are prepared as described in Example 1 by reacting 6-bromo-4-chloro-3-nitro-quinoline (Example 1c) with 1-(4-amino-phenyl)-pyrrolidin-2-one (Example 121a) as in Example 1e, and with a cyclisation reaction as in Example 1g, Example 32, Example 45 or Example 64.

TABLE 29

| | | ES-MS | tᵣₑₜ |
|---|---|---|---|
| Example | Compound name | (M + H)⁺ | [min] |
| 121 | 1-[4-(8-Pyridin-3-ylethynyl-imidazo[4,5-c]quinolin-1-yl)- | 430 | 2.56 |
| | phenyl]-pyrrolidin-2-one | | Grad 2 |
| 122 | 1-[4-(2-Methyl-8-pyridin-3-ylethynyl-imidazo[4,5-c]quinolin- | 444 | 2.60 |
| | 1-yl)-phenyl]-pyrrolidin-2-one | | Grad 2 |
| 123 | 1-[4-(2-Methoxy-8-pyridin-3-ylethynyl-imidazo[4,5- | 460 | 2.66 |
| | c]quinolin-1-yl)-phenyl]-pyrrolidin-2-one | | Grad 2 |
| 124 | 1-[4-(2-Dimethylamino-8-pyridin-3-ylethynyl-imidazo[4,5- | 473 | 2.70 |
| | c]quinolin-1-yl)-phenyl]-pyrrolidin-2-one | | Grad 2 |

### Example 121a

### 1-(4-Amino-phenyl)-pyrrolidin-2-one

The title compound is obtained as described in Example 48a starting with 1-(4-nitro-phenyl)-1-pyrrolidin-2-one (Acros, Basel, CH); ES-MS: 177 (M+H)⁺; analytical HPLC: tᵣₑₜ=2.71 minutes (Grad 2).

The following compounds (see Table 30) are prepared as described in Example 1 by reacting 6-bromo-4-chloro-3-nitro-quinoline (Example 1c) with 5-amino-2-(2-oxo-pyrrolidin-1-yl)-benzonitrile (Example 125a) as in Example 1e, and with a cyclisation reaction as in Example 1g, Example 32, Example 45 or Example 64.

TABLE 30

| | | ES-MS | tᵣₑₜ |
|---|---|---|---|
| Example | Compound name | (M + H)⁺ | [min] |
| 125 | 2-(2-Oxo-pyrrolidin-1-yl)-5-(8-pyridin-3-ylethynyl- | 455 | 2.47 |
| | imidazo[4,5-c]quinolin-1-yl)-benzonitrile | | Grad 2 |
| 126 | 5-(2-Methyl-8-pyridin-3-ylethynyl-imidazo[4,5-c]quinolin-1- | 469 | 2.48 |
| | yl)-2-(2-oxo-pyrrolidin-1-yl)-benzonitrile | | Grad 2 |
| 127 | 5-(2-Methoxy-8-pyridin-3-ylethynyl-imidazo[4,5-c]quinolin- | 485 | 2.55 |
| | 1-yl)-2-(2-oxo-pyrrolidin-1-yl)-benzonitrile | | Grad 2 |
| 128 | 5-(2-Dimethylamino-8-pyridin-3-ylethynyl-imidazo[4,5- | 498 | 2.56 |
| | c]quinolin-1-yl)-2-(2-oxo-pyrrolidin-1-yl)-benzonitrile | | Grad 2 |

### Example 125a

### 5-Amino-2-(2-oxo-pyrrolidin-1-yl)-benzonitrile

The title compound is obtained as described in Example 48a starting with 5-nitro-2-(2-oxo-1-pyrrolidin-1-yl)-benzonitrile (Example 125b); ES-MS: 202 (M+H)⁺; analytical HPLC: tᵣₑₜ=2.09 minutes (Grad 2).

### Example 125b

### 5-Nitro-2-(2-oxo-pyrrolidin-1-yl)-benzonitrile

The title compound is obtained as described in Example 117b starting with 2-fluoro-5-nitro benzonitrile (Aldrich, Buchs, Switzerland); ES-MS: 232 (M+H)⁺; analytical HPLC: tᵣₑₜ=2.80 minutes (Grad 2).

The following compounds (see Table 31) are prepared as described in Example 1 by reacting 6-bromo-4-chloro-3-nitro-quinoline (Example 1c) with 3-(4-amino-2-fluoro-phenyl)-oxazolidin-2-one (Example 129a) as in Example 1e, and with a cyclisation reaction as in Example 1g, Example 32, Example 45 or Example 64.

TABLE 31

| | | ES-MS | tᵣₑₜ |
|---|---|---|---|
| Example | Compound name | (M + H)⁺ | [min] |
| 129 | 3-[2-Fluoro-4-(8-pyridin-3-ylethynyl-imidazo[4,5-c]quinolin- | 450 | 2.51 |
| | 1-yl)-phenyl]-oxazolidin-2-one | | Grad 2 |
| 130 | 3-[2-Fluoro-4-(2-methyl-8-pyridin-3-ylethynyl-imidazo[4,5- | 464 | 2.52 |
| | c]quinolin-1-yl)-phenyl]-oxazolidin-2-one | | Grad 2 |
| 131 | 3-[2-Fluoro-4-(2-methoxy-8-pyridin-3-ylethynyl- | 480 | 2.60 |
| | imidazo[4,5-c]quinolin-1-yl)-phenyl]-oxazolidin-2-one | | Grad 2 |
| 132 | 3-[4-(2-Dimethylamino-8-pyridin-3-ylethynyl-imidazo[4,5- | 493 | 2.62 |
| | c]quinolin-1-yl)-2-fluoro-phenyl]-oxazolidin-2-one | | Grad 2 |

### Example 129a

### 3-(4-Amino-2-fluoro-phenyl)-oxazolidin-2-one

The title compound is obtained as described in Example 48a starting with 3-(2-fluoro-4-nitro phenyl)-oxazolidin-2-one (Example 129b); ES-MS: 197 (M+H)⁺; analytical HPLC: tᵣₑₜ=1.66 minutes (Grad 2).

### Example 129b

### 3-(2-Fluoro-4-nitro-phenyl)-oxazolidin-2-one

The title compound is obtained as described in Example 117b starting with 2-oxazolidinone (Fluka, Buchs, Switzerland); ES-MS: 225 (M−H)⁻; analytical HPLC: tᵣₑₜ=2.90 minutes (Grad 2).

The following compounds (see Table 32) are prepared as described in Example 1 by reacting 6-bromo-4-chloro-3-nitro-quinoline (Example 1c) with 3-(4-amino-phenyl)-oxazolidin-2-one (Example 132a) as in Example 1e, and with a cyclisation reaction as in Example 1g, Example 32 or Example 45.

TABLE 32

| | | ES-MS | tᵣₑₜ |
|---|---|---|---|
| Example | Compound name | (M + H)⁺ | [min] |
| 133 | 3-[4-(8-Pyridin-3-ylethynyl-imidazo[4,5-c]quinolin-1-yl)- | 432 | 2.50 |
| | phenyl]-oxazolidin-2-one | | Grad 2 |
| 134 | 3-[4-(2-Methyl-8-pyridin-3-ylethynyl-imidazo[4,5-c]quinolin- | 446 | 2.52 |
| | 1-yl)-phenyl]-oxazolidin-2-one | | Grad 2 |
| 135 | 3-[4-(2-Methoxy-8-pyridin-3-ylethynyl-imidazo[4,5- | 462 | 2.60 |
| | c]quinolin-1-yl)-phenyl]-oxazolidin-2-one | | Grad 2 |

### Example 133a

### 3-(4-Amino-phenyl)-oxazolidin-2-one

The title compound is obtained as described in Example 48a starting with 3-(4-nitro-phenyl)-oxazolidin-2-one (Example 133b); ES-MS: 179 (M+H)⁺; analytical HPLC: tᵣₑₜ=1.46 minutes (Grad 2).

### Example 133b

### 3-(4-Nitro-phenyl)-oxazolidin-2-one

The title compound is obtained as described in Example 117b starting with 2-oxazolidinone (Fluka, Buchs, Switzerland) and 4-fluoro-nitrobenzene (Aldrich, Buchs, Switzerland); analytical HPLC: tᵣₑₜ=2.98 minutes (Grad 2).

The following compounds (see Table 33) are prepared as described in Example 1 by reacting 6-bromo-4-chloro-3-nitroquinoline (Example 1c) with 1-(4-amino-2-fluoro-phenyl)-pyrrolidine-2,5-dione (Example 136a) as in Example 1e, and with a cyclisation reaction as in Example 1g, Example 32, Example 45 or Example 64.

TABLE 33

| | | ES-MS | tᵣₑₜ |
|---|---|---|---|
| Example | Compound name | (M + H)⁺ | [min] |
| 136 | 1-[2-Fluoro-4-(8-pyridin-3-ylethynyl-imidazo[4,5-c]quinolin- | 462 | 5.65 |
| | 1-yl)-phenyl]-pyrrolidine-2,5-dione | | Grad 3 |
| 137 | 1-[2-Fluoro-4-(2-methyl-8-pyridin-3-ylethynyl-imidazo[4,5- | 476 | 5.71 |
| | c]quinolin-1-yl)-phenyl]-pyrrolidine-2,5-dione | | Grad 3 |
| 138 | 1-[2-Fluoro-4-(2-methoxy-8-pyridin-3-ylethynyl- | 492 | 5.50 |
| | imidazo[4,5-c]quinolin-1-yl)-phenyl]-pyrrolidine-2,5-dione | | Grad 3 |
| 139 | 1-[4-(2-Dimethylamino-8-pyridin-3-ylethynyl-imidazo[4,5- | 505 | 5.57 |
| | c]quinolin-1-yl)-2-fluoro-phenyl]-pyrrolidine-2,5-dione | | Grad 3 |

### Example 136a

### 1-(4-Amino-2-fluorophenyl)-pyrrolidine-2,5-dione

The title compound is obtained by reduction of 1-(2-fluoro-4-nitro-phenyl)-pyrrolidine-2,5-dione (Example 136b) as described in Example 58a. ES-MS: 209.2 (M+H)⁺; analytical HPLC: tᵣₑₜ=4.69 minutes (Grad 3).

### Example 136b

### 1-(2-fluoro-4-nitro-phenyl)-pyrrolidine-2,5-dione

The title compound is obtained as described in Example 50b using 1,2-difluoro-4-nitro-benzene (Aldrich, Buchs, Switzerland) and pyrrolidine-2,5-dione (Aldrich, Buchs, Switzerland) in DMSO at 100° C. ES-MS: 238.1 (M−H)⁺; analytical HPLC: tᵣₑₜ=6.52 minutes (Grad 3).

The following compounds (see Table 34) are prepared as described in Example 1 by reacting 6-bromo-4-chloro-3-nitro-quinoline (Example 1c) with 4-(4-amino-2-fluoro-phenyl)-piperazine-1-carboxylic acid tert-butyl ester (Example 74a), and with a cyclisation reaction as in Example 1g, Example 45 or Example 64.

TABLE 34

| | | ES-MS | tᵣₑₜ |
|---|---|---|---|
| Example | Compound name | (M + H)⁺ | [min] |
| 140 | 1-[2-Fluoro-4-(8-pyridin-3-ylethynyl-imidazo[4,5-c]quinolin- | 449 | 2.34 |
| | 1-yl)-phenyl]-pyrrolidine-2,5-dione | | Grad 2 |
| 141 | 1-[2-Fluoro-4-(2-methyl-8-pyridin-3-ylethynyl-imidazo[4,5- | 479 | 2.38 |
| | c]quinolin-1-yl)-phenyl]-pyrrolidine-2,5-dione | | Grad 2 |
| 142 | 1-[2-Fluoro-4-(2-methoxy-8-pyridin-3-ylethynyl- | 492 | 2.43 |
| | imidazo[4,5-c]quinolin-1-yl)-phenyl]-pyrrolidine-2,5-dione | | Grad 2 |

The following compounds (see Table 35) are prepared as described in Example 1 by reacting 6-bromo-4-chloro-3-nitro-quinoline (Example 1c) with 4-(4-amino-2-fluoro-phenyl)-piperazin-2-one (Example 143a) as in Example 1e, with cyclisation reaction as in Example 1g or Example 32, and with or without a subsequent introduction of an ethyl (Example 145a) or a methyl group (Example 147a).

TABLE 35

| | | ES-MS | tᵣₑₜ |
|---|---|---|---|
| Example | Compound name | (M + H)⁺ | [min] |
| 143 | 4-[2-Fluoro-4-(8-pyridin-3-ylethynyl-imidazo[4,5-c]quinolin- | 463 | 2.48 |
| | 1-yl)-phenyl]-piperazin-2-one | | Grad 2 |
| 144 | 1-Ethyl-4-[2-fluoro-4-(8-pyridin-3-ylethynyl-imidazo[4,5- | 477 | 2.50 |
| | c]quinolin-1-yl)-phenyl]-piperazin-2-one | | Grad 2 |
| 145 | 1-Ethyl-4-[2-fluoro-4-(8-pyridin-3-ylethynyl-imidazo[4,5- | 491 | 2.71 |
| | c]quinolin-1-yl)-phenyl]-piperazin-2-one | | Grad 2 |
| 146 | 1-Ethyl-4-[2-fluoro-4-(2-methyl-8-pyridin-3-ylethynyl- | 505 | 2.73 |
| | imidazo[4,5-c]quinolin-1-yl)-phenyl]-piperazin-2-one | | Grad 2 |
| 147 | 4-[2-Fluoro-4-(8-pyridin-3-ylethynyl-imidazo[4,5-c]quinolin- | 477 | 2.60 |
| | 1-yl)-phenyl]-1-methyl-piperazin-2-one | | Grad 2 |
| 148 | 4-[2-Fluoro-4-(2-methyl-8-pyridin-3-ylethynyl-imidazo[4,5- | 491 | 2.62 |
| | c]quinolin-1-yl)-phenyl]-1-methyl-piperazin-2-one | | Grad 2 |

### Example 143a

### 4-(4-Amino-2-fluoro-phenyl)-piperazin-2-one

The title compound is obtained as described in Example 48a starting with 4-(2-fluoro-4-nitro-phenyl)-piperazin-2-one (Example 143b); ES-MS: 210 (M+H)⁺; analytical HPLC: tᵣₑₜ=1.41 minutes (Grad 2).

### Example 143b

### 4-(2-Fluoro-4-nitro-phenyl)-piperazin-2-one

The title compound is obtained as described in Example 50b starting with 3,4-difluoro-nitrobenzene (Fluka, Buchs, Switzerland) and piperazin-2-one (Avocado, Heysham, UK); ES-MS: 238 (M−H)⁻.

### Example 145a

### 4-[4-(8-Bromo-imidazo[4,5-c]quinolin-1-yl)-2-fluoro-phenyl]-1-ethyl-piperazin-2-one

200 mg (0.454 mmol) 4-[4-(8-bromo-imidazo[4,5-c]quinolin-1-yl)-2-fluoro-phenyl]-piperazin-2-one (intermediate in Example 143; ES-MS: 440, 442 (M+H)⁺, Br pattern) and 2 ml DMF are treated under Ar with 22 mg (0.5 mmol) of 55% NaH in oil. The reaction mixture is stirred for 2 h at RT, then 85 mg (0.545 mmol) iodoethane (Fluka, Buchs, CH) are added. The reaction mixture is stirred 12 h at RT. After this time, the reaction mixture is quenched with saturated aqueous NaHCO₃ and extracted with EtOAc. The organic layer is washed with brine (3×) and dried over MgSO₄, filtered and evaporated to dryness. The residue is preabsorbed on silica gel and purified by flash chromatography on silica gel (CH₂Cl₂-MeOH 1:0 to 92:8) to provide the title compound: ES-MS: 468, 470 (M+H)⁺; analytical HPLC: tᵣₑₜ=2.92 minutes (Grad 2).

### Example 147a

### 4-[4-(8-Bromo-imidazo[4,5-c]quinolin-1-yl)-2-fluoro-phenyl]-1-methyl-piperazin-2-one

The title compound is obtained as described in Example 145a using iodomethane (Fluka, Buchs, CH); ES-MS: 454, 456 (M+H)⁺, Br pattern; analytical HPLC: tᵣₑₜ=2.76 minutes (Grad 2).

The following compounds (see Table 69) are prepared as described in Example 1 by reacting 6-bromo-4-chloro-3-nitro-quinoline (Example 1c) with 5-amino-2-cyanomethyl-benzonitrile (Example 149a) as in Example 1e, and with or without a subsequent introduction of two methyl groups (Example 150a).

TABLE 36

| | | ES-MS | tᵣₑₜ |
|---|---|---|---|
| Example | Compound name | (M + H)⁺ | [min] |
| 149 | 2-Cyanomethyl-5-(2-methyl-8-pyridin-3-ylethynyl- | 425 | 2.52 |
| | imidazo[4,5-c]quinolin-1-yl)-benzonitrile | | Grad 2 |
| 150 | 2-(Cyano-dimethyl-methyl)-5-(2-methyl-8-pyridin-3- | 453 | 2.75 |
| | ylethynyl-imidazo[4,5-c]quinolin-1-yl)-benzonitrile | | Grad 2 |

### Example 149a

### 5-Amino-2-cyanomethyl-benzonitrile

The title compound is obtained as described in Example 48a starting with cyano-(2-cyano-4-nitro-phenyl)-acetic acid benzyl este (Example 149b); ES-MS: 157 (M−H)⁻, Br pattern; analytical HPLC: tᵣₑₜ=2.10 minutes (Grad 2).

### Example 149b

### Cyano-(2-cyano-4-nitro-phenyl)-acetic acid benzyl ester

1.0 g (6.02 mmol) of 2-fluoro-5-nitro-benzonitrile (Aldrich, Buchs, CH), 1.15 g (8.31 mmol) of finely-powdered K₂CO₃, 10 mg (0.06 mmol) of KI and 1.16 g (6.62 mmol) of benzyl cyanoacetate in 6 mL DMF are stirred under Ar for 5 h at 50° C. and 1 h at 100° C. The reaction mixture is quenched with H₂O and extracted with EtOAc (2×). The combined organic layers are washed with brine (3×), dried over MgSO₄, filtered and evaporated to dryness to provide the title compound: ES-MS: 320 (M−H)⁻; analytical HPLC: t=et 3.92 minutes (Grad 2).

### Example 150a

### 5-(8-Bromo-2-methyl-imidazo[4,5-c]quinolin-1-yl)-2-(cyano-dimethyl-methyl)-benzonitrile

830 mg (2.06 mmol) of 5-(8-bromo-2-methyl-imidazo[4,5-c]quinolin-1-yl)-2-cyanomethyl-benzonitrile (intermediate in Example 149; ES-MS: 402, 404 (M+H)⁺, Br pattern) in 20 ml of DMF are treated under Ar with 198 mg (2.27 mmol) of 55% NaH in oil. The reaction mixture is stirred for 1 h at RT and then is cooled with an ice-bath and 142 ul (2.27 mmol) iodomethane (Fluka, Buchs, CH) are added. The reaction mixture is stirred for 1 h at RT, then are added 198 mg (2.27 mmol) 55% NaH in oil. The reaction mixtures is stirred for 1 h at RT, then is cooled with an ice-bath and 142 ul (2.27 mmol) iodomethane are added and the reaction mixture is stirred for 1 h at RT. After this time, the reaction mixture is quenched with brine and is extracted with EtOAc. The organic layer is washed with brine, dried with MgSO₄, filtered and concentrated in vacuo. The residue is purified by medium-pressure liquid chromatography to provide the title compound: ES-MS: 430, 432 (M+H)⁺; analytical HPLC: tᵣₑₜ=3.09 minutes (Grad 2).

The following compounds (see Table 37) are prepared as described in Example 1 by reacting 6-bromo-4-chloro-3-nitro-quinoline (Example 1c) with the appropriate aniline as in Example 1e, and with cyclisation as described in Example 151a and subsequent methylation as described in Example 151b.

Example 151 4-fluoro-aniline (Fluka, Buchs, CH);

Example 152 4-ethyl-aniline (Fluka, Buchs, CH);

Example 153 3-methoxy-aniline (Fluka, Buchs, CH);

Example 154 4-methoxy-aniline (Fluka, Buchs, CH);

Example 155 3,4,5-trimethoxy-aniline (Fluka, Buchs, CH);

Example 156 (2-(4-amino-phenyl)-2-methyl-propionitrile (Example 107b);

Example 157 3-(4-amino-phenyl)-oxazolidin-2-one (Example 133a);

Example 158 3-(4-amino-2-fluoro-phenyl)-oxazolidin-2-one (Example 129a);

Example 159 4-(4-amino-phenyl)-piperazine-1-carboxylic acid tert-butyl ester (Example 73a);

Example 160 4-(4-amino-2-fluoro-phenyl)-piperazine-1-carboxylic acid tert-butyl Example 74a); and

Example 161 (4-amino-phenyl)-carbamic acid tert-butyl ester (Fluka, Buchs, CH).

TABLE 37

| | | ES-MS | tᵣₑₜ |
|---|---|---|---|
| Example | Compound name | (M + H)⁺ | [min] |
| 151 | 1-(4-Fluoro-phenyl)-3-methyl-8-pyridin-3-ylethynyl-1,3- | 395 | 2.62 |
| | dihydro-imidazo[4,5-c]quinolin-2-one | | Grad 2 |
| 152 | 1-(4-Ethyl-phenyl)-3-methyl-8-pyridin-3-ylethynyl-1,3- | 405 | 2.95 |
| | dihydro-imidazo[4,5-c]quinolin-2-one | | Grad 2 |
| 153 | 1-(3-Methoxy-phenyl)-3-methyl-8-pyridin-3-ylethynyl-1,3- | 407 | 2.65 |
| | dihydro-imidazo[4,5-c]quinolin-2-one | | Grad 2 |
| 154 | 1-(4-Methoxy-phenyl)-3-methyl-8-pyridin-3-ylethynyl-1,3- | 407 | 2.64 |
| | dihydro-imidazo[4,5-c]quinolin-2-one | | Grad 2 |
| 155 | 3-Methyl-8-pyridin-3-ylethynyl-1-(3,4,5-trimethoxy-phenyl)- | 467 | 2.59 |
| | 1,3-dihydro-imidazo[4,5-c]quinolin-2-one | | Grad 2 |
| 156 | 2-Methyl-2-[4-(3-methyl-2-oxo-8-pyridin-3-ylethynyl-2,3- | 444 | 2.83 |
| | dihydro-imidazo[4,5-c]quinolin-1-yl)-phenyl]-propionitrile | | Grad 2 |
| 157 | 3-Methyl-1-[4-(2-oxo-oxazolidin-3-yl)-phenyl]-8-pyridin-3- | 462 | 2.48 |
| | ylethynyl-1,3-dihydro-imidazo[4,5-c]quinolin-2-one | | Grad 2 |
| 158 | 1-[3-Fluoro-4-(2-oxo-oxazolidin-3-yl)-phenyl]-3-methyl-8- | 480 | 2.53 |
| | pyridin-3-ylethynyl-1,3-dihydro-imidazo[4,5-c]quinolin-2- | | Grad 2 |
| | one | | |
| 159 | 3-Methyl-1-(4-piperazin-1-yl-phenyl)-8-pyridin-3-ylethynyl- | 461 | 2.29 |
| | 1,3-dihydro-imidazo[4,5-c]quinolin-2-one | | Grad 2 |
| 160 | 1-(3-Fluoro-4-piperazin-1-yl-phenyl)-3-methyl-8-pyridin-3- | 479 | 2.34 |
| | ylethynyl-1,3-dihydro-imidazo[4,5-c]quinolin-2-one | | Grad 2 |
| 161 | 3-Methyl-1-(4-methylamino-phenyl)-8-pyridin-3-ylethynyl- | 406 | 2.52 |
| | 1,3-dihydro-imidazo[4,5-c]quinolin-2-one | | Grad 2 |

### Example 151a

### 8-Bromo-1-(4-fluoro-phenyl)-1,3-dihydro-imidazo[4,5-c]quinolin-2-one

To a solution of 1.63 g (4.19 mmol) of 6-bromo-N*4*-(4-fluoro-phenyl)-quinoline-3,4-diamine (ES-MS: 332, 334 (M+H)⁺, Br pattern; analytical HPLC: tᵣₑₜ=3.10 minutes (Grad 2)) and 596 mg (5.89 mmol) of triethylamine in 50 ml of CH₂Cl₂ cooled with an ice-bath are added, under argon and over 10 min, 1.07 g (5.4 mmol) of trichloromethyl chloroformate (Fluka, Buchs, CH) in 50 ml CH₂Cl₂. The reaction mixture is stirred for 30 min at 0° C. After this time, the reaction mixture is quenched with brine and extracted with CH₂Cl₂ (3×). The combined organic layers are washed with brine (3×), dried over Na₂SO₄, filtered and evaporated to dryness to provide the title compound: ES-MS: 358, 360 (M+H)⁺, Br pattern; analytical HPLC: tᵣₑₜ=2.92 minutes (Grad 2).

### Example 151b

### 8-Bromo-1-(4-fluoro-phenyl)-3-methyl-1,3-dihydro-imidazo[4,5-c]quinolin-2-one

1.51 g (4.22 mmol) of 8-bromo-1-(4-fluoro-phenyl)-1,3-dihydro-imidazo[4,5-c]quinolin-2-one (Example 151a), 136 mg (0.422 mmol) of tetrabutylammonium bromide, 898 mg (6.32 mmol) of iodomethane (Fluka, Buchs, CH) in 100 ml of CH₂Cl₂ are treated with a solution of 253 mg (6.32 mmol) of NaOH in 50 ml H₂O. The reaction mixture is stirred 13 h at RT. After this time, the reaction mixture is extracted with CH₂Cl₂(2×). The combine organic layers are washed with brine, dried over Na₂SO₄, filtered and concentrated in vacuo. The residue is preabsrobed on silica gel and is purified by flash chromatography (CH₂Cl₂-MeOH 1:0 to 93:7) to provide the title compound: ES-MS: 372, 374 (M+H)⁺, Br pattern; analytical HPLC: tᵣₑₜ=3.01 minutes (Grad 2).

### Example 162

### N-Methyl-N-[4-(3-methyl-2-oxo-8-pyridin-3-ylethynyl-2,3-dihydro-imidazo[4,5-c]quinolin-1-yl)-phenyl]-acetamide

110 mg (0.214 mmol) of 3-methyl-1-(4-methylamino-phenyl)-8-pyridin-3-ylethynyl-1,3-dihydro-imidazo[4,5-c]quinolin-2-one-3 HCl (Example 161 HCl salt) and 108 mg (1.07 mmol) of triethylamine in 2 ml of CH₂Cl₂ are stirred 15 min. The reaction mixture is treated with 25.4 mg (0.324 mmol) of acetyl chloride (Fluka, Buchs, CH). The reaction mixture is stirred 3 h at RT. After this time, 16.6 mg (0.211 mmol) of acetyl chloride are added and the reaction mixture is stirred for 2 at RT, then the reaction mixture is quenched with brine and is extracted with CH₂Cl₂. The combine organic layers are washed with sat. aqueous NaHCO₃, with brine, dried over Na₂SO₄, filtered and concentrated in vacuo. The residue is pre-absorbed on silica gel and is purified by flash chromatography (CH₂Cl₂-MeOH 1:0 to 93:7) to provide the title compound: ES-MS: 448 (M+H)⁺; analytical HPLC: tᵣₑₜ=2.48 minutes (Grad 2).

### Example 163

### Inhibition of PDK1 Kinase by Compounds of the Present Invention

Activity determinations of compounds of the preceding examples, using the testing method described above, with the following test compounds of formula (I) exhibit the following IC₅₀ values for PDK1 inhibition:

| Example | IC₅₀ μM |
|---|---|
| 3 | A |
| 4 | A |
| 8 | A |
| 9 | A |
| 10 | A |
| 11 | A |
| 12 | A |
| 13 | A |
| 23 | B |
| 26 | B |
| 27 | B |
| 28 | A |
| 29 | B |
| 30 | B |
| 32 | A |
| 33 | B |
| 34 | A |
| 35 | A |
| 38 | A |

| | |
|---|---|
| Letter | IC₅₀ range class |
| A | ≦0.5 μM |
| B | more than 0.5 μM up to 1 μM |

### Example 164

### Tablets 1 Comprising Compounds of the Formula (I)

Tablets, comprising, as active ingredient, 50 mg of any one of the compounds of formula (I) mentioned in the preceding Examples 1-162 of the following composition are prepared using routine methods:

Composition:

| | |
|---|---|
| Active Ingredient | 50 mg |
| Wheat starch | 60 mg |
| Lactose | 50 mg |
| Colloidal silica | 5 mg |
| Talcum | 9 mg |
| Magnesium stearate | 1 mg |
| | 175 mg |

Manufacture: The active ingredient is combined with part of the wheat starch, the lactose and the colloidal silica and the mixture pressed through a sieve. A further part of the wheat starch is mixed with the 5-fold amount of water on a water bath to form a paste and the mixture made first is kneaded with this paste until a weakly plastic mass is formed.

The dry granules are pressed through a sieve having a mesh size of 3 mm, mixed with a pre-sieved mixture (1 mm sieve) of the remaining corn starch, magnesium stearate and talcum and compressed to form slightly biconvex tablets.

### Example 165

### Tablets 2 Comprising Compounds of the Formula (I)

Tablets, comprising, as active ingredient, 100 mg of any one of the compounds of formula (I) of Examples 1-162 are prepared with the following composition, following standard procedures:

Composition:

| | |
|---|---|
| Active Ingredient | 100 mg |
| Crystalline lactose | 240 mg |
| Avicel | 80 mg |
| PVPPXL | 20 mg |
| Aerosil | 2 mg |
| Magnesium stearate | 5 mg |
| | 447 mg |

Manufacture: The active ingredient is mixed with the carrier materials and compressed by means of a tabletting machine (Korsch EKO, Stempeldurchmesser 10 mm).

### Example 166

### Capsules

Capsules, comprising, as active ingredient, 100 mg of any one of the compounds of formula (I) given in Examples 1-162, of the following composition are prepared according to standard procedures:

Composition:

| | | |
|---|---|---|
| Active Ingredient | 100 | mg |
| Avicel | 200 | mg |
| PVPPXL | 15 | mg |
| Aerosil | 2 | mg |
| Magnesium stearate | 1.5 | mg |
| | 318.5 | mg |

Manufacturing is done by mixing the components and filling them into hard gelatine capsules, size 1.

What is claimed is:

1. A compound according to formula (I) 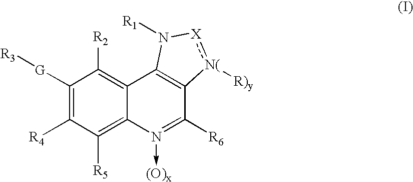
wherein
each of x and y is Independently of the other 0 or 1,
R₁ is an organic moiety that can be bound to nitrogen,
X is C═O or C═S with the proviso that then the dashed line bonding X to N is absent, so that X is bound to the adjacent N via a single bond the with the proviso that then y is 1 and R is hydrogen or an organic moiety that can be bound to nitrogen; or
X is (CR₇), wherein R₇ is hydrogen or an organic or inorganic moiety with the proviso that then the dashed line bonding X to N is a bond, so that X is bound to the adjacent N via a double bond, and with the proviso that then y is zero or y is 1 and then —R is →O;
G is unsubstituted or substituted alkenylene, unsubstituted or substituted alkynylene; and
each of R₂, R₃, R₄, R₅ and R₆, independently of the others, is hydrogen, an organic moiety or an inorganic moiety;
or a pharmaceutically acceptable salt thereof.

2. A compound according to claim 1,
wherein
each of x and y is, independently of the other, 0 or 1;
R₁ is substituted or unsubstituted aryl or heteroaryl, especially phenyl, which is substituted with up to 4, preferably up to 2 substituents, wherein the substituents are the same or different and are independently selected from halo (e.g. Cl or F); cyano; cyano lower alkyl (e.g. cyanomethyl, cyanoethyl and cyanopropyl); lower alkyl; lower alkoxy; amino; amino-lower alkyl; amino-lower alkoxy; amino-lower alkyl sulfanyl or thiol-lower alkyl; wherein the amino group can be mono or disubstituted, [e.g. —(C₁-C₇)NR₈R₉ or —O—(C₁-C₇)NR₈R₉, wherein R₈ and R₉ can be the same or different and are independently H, lower alkyl (e.g. methyl, ethyl or propyl), lower cycloalkyl (e.g. cyclopropyl) or R₈ and R₉, together with the N atom, form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl)]; amino-carbonyl-lower alkyl (e.g. R₈R₉—N—C(O)—CH₂—, wherein R₈ and R₉ are as defined above); heterocyclyl; heterocyclyl-lower alkyl; heterocyclyl-lower alkoxy or heterocyclyl-lower alkanesulfanyl wherein the heterocyclyl is a 3- to 8-membered heterocyclic ring containing 1-4 nitrogen, oxygen or sulfur atoms (e.g. imidazolyl, imidazolinyl, pyrrolidinyl, morpholinyl, azetidinyl, pyridyl, piperidino, piperidyl, piperazinyl or lower alkyl-piperazinyl); wherein alkyl may be linear or cyclic (e.g. cyclopropyl) and the alkyl in any of the substituents above may optionally be substituted with —NR₈R₉, wherein R₈ and R₉ are as defined above;
X is C═O or C═S with the proviso that then the dashed line bonding X to N is absent, so that X is bound to the adjacent N via a single bond and with the proviso that then y is 1 and R is hydrogen or an organic moiety that can be bound to nitrogen; or
X is (CR₇) wherein R₇ is hydrogen or an organic moiety, such as C₁-C₇lower alkyl; amino or amino-lower alkyl; wherein alkyl may be unsubstituted or substituted with halo (e.g. methyl, ethyl, propyl, trifluoromethyl); lower alkoxy (e.g. methoxy); or cycloalkyl (e.g. cyclopropyl); with the proviso that then the dashed line bonding X to N is a bond, so that X is bound to the adjacent N via a double bond, and with the proviso that then y is zero, or y is 1 and then —R is →O;
G is unsubstituted or substituted alkenylene (e.g. ethenylene), unsubstituted or substituted alkynylene (e.g. ethynylene);
R₂ is hydrogen;
R₃ is hydrogen; lower alkyl; halo (e.g. fluoro, chloro or bromo); lower alkoxy (e.g. methoxy); or unsubstituted or substituted C₅-C₁₄aryl (e.g. phenyl, hydroxyphenyl, methoxyphenyl or aminosulfonyl-phenyl or benzo[1,3]dioxolo); or a heteroaryl being unsubstituted or substituted by one or more, especially 1-4 substituents; pyridyl (or an N-oxide of pyridyl) which is unsubstituted or substituted by one to two radicals selected from the group consisting of lower alkyl (e.g. methyl); lower alkoxy (e.g. methoxy); halo (e.g. fluoro); or —NR₈R₉, wherein R₈ and R₉ can be the same or different and are independently H, lower alkyl (e.g. methyl, ethyl or propyl); lower cycloalkyl (e.g. cyclopropyl); or the R₈ and R₉ can, with the N atom, form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl);
R₄ is hydrogen or halo (e.g. F or Cl);
R₅ is hydrogen; and
R₆ is hydrogen; amino; amino-lower alkyl or alkylamido (e.g. methylamido —NHC(O)—CH₃);
or a pharmaceutically acceptable salt thereof.

3. A compound of formula (I) according to claim 1 wherein
each of x and y is, independently of the other, 0 or 1;
R₁ is substituted or unsubstituted phenyl where the phenyl is substituted with up to 4, preferably up to 2 substituents, wherein the substituents are the same or different and are independently selected from halo (e.g. Cl or F); cyano; cyano lower alkyl (e.g. cyanomethyl, cyanoethyl and cyanopropyl); lower alkyl; lower alkoxy; N-lower alkyl amino alkyl (e.g. methyl aminoethyl, cyclopropyl aminoethyl); N,N-di-lower alkyl amino alkyl; methoxy amino; methoxy N-methyl amino; amino; amino-lower alkyl; amino-lower alkoxy; azetidinyl lower alkyl; pyrrolidinyl; N-lower alkyl sulfonamide alkyl (e.g. CH₃—NH₂—S(O)₂alkyl); amino-lower alkyl sulfanyl or thiol-lower alkyl; wherein the amino group can be mono or disubstituted [e.g. —(C₁-C₇)NR₈R₉ or —O—(C₁-C₇)NR₈R₉, wherein R₈ and R₉ can be the same or different and are independently H; lower alkyl (e.g. methyl, ethyl or propyl); lower cycloalkyl (e.g. cyclopropyl); or R₈ and R₉ together with the N atom form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl)]; amino-carbonyl-lower alkyl (e.g. R₈R₉—N—C(O)—CH₂—, wherein R₈ and R₉ are as defined above); heterocyclyl; heterocyclyl-lower alkyl; lower alkyl piperazinyl-lower alkyl; heterocyclyl-lower alkoxy or heterocyclyl-lower alkanesulfanyl wherein the heterocyclyl is a 3- to 8-membered heterocyclic ring containing 1-4 nitrogen, oxygen or sulfur atoms (e.g. imidazolyl, imidazolinyl, pyrrolidinyl, morpholinyl, azetidinyl, pyridyl, piperidino, piperidyl, piperazinyl or lower alkyl-piperazinyl); substituted heterocyclyls such as pyrrolidin-2-one, oxazolidin-2-one, pyrrolidine-2,5-dione, piperazine-2-one and oxo-oxazolidinyl; wherein alkyl may be linear or cyclic (e.g. cyclopropyl) and the alkyl in any of the substituents above may optionally be substituted with —NR₈R₉, wherein R₈ and R₉ are as defined above;
X is C═O or C═S with the proviso that then the dashed line bonding X to N is absent, so that X is bound to the adjacent N via a single bond and with the proviso that then y is 1 and R is hydrogen or an organic moiety that can be bound to nitrogen; or
X is (CR₇), wherein R₇ is hydrogen or an organic moiety, such as C₁-C₇lower alkyl; amino; amino-lower alkyl; wherein the alkyl may be unsubstituted or substituted with halo (e.g. methyl, ethyl, propyl, trifluoromethyl); lower alkoxy (e.g. methoxy); or cycloalkyl (e.g. cyclopropyl); with the proviso that then the dashed line bonding X to N is a bond, so that X is bound to the adjacent N via a double bond, and with the proviso that then y is zero, or y is 1 and then —R is →O;
G is unsubstituted or substituted alkenylene (e.g. ethenylene), unsubstituted or substituted alkynylene (e.g. ethynylene);
R₂ is hydrogen;
R₃ is hydrogen; lower alkyl; halo (e.g. fluoro, chloro or bromo); lower alkoxy (e.g. methoxy); or unsubstituted or substituted C₅-C₁₄aryl (e.g. phenyl, hydroxyphenyl, methoxyphenyl or aminosulfonyl-phenyl or benzo[1,3]dioxolo); or a heteroaryl being unsubstituted or substituted by one or more, especially 1-4, substituents independently selected from the group consisting of the substituents defined above under “substituted”; especially being pyridyl (or an N-oxide of pyridyl) which is unsubstituted or substituted by one to two radicals selected from the group consisting of lower alkyl (e.g. methyl); lower alkoxy (e.g. methoxy); halo (e.g. fluoro); or —NR₈R₉, wherein R₈ and R₉ can be the same or different and are independently H, lower alkyl (e.g. methyl, ethyl or propyl); lower cycloalkyl (e.g. cyclopropyl); or the R₈ and R₉ can, with the N atom, form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl);
R₄ is hydrogen or halo, (e.g. F or Cl);
R₅ is hydrogen; and
R₆ is hydrogen; amino; amino-lower alkyl or alkylamido (e.g. methylamido —NHC(O)—CH₃);
or a pharmaceutically acceptable salt thereof as such, or
especially for use in the diagnostic or therapeutic treatment of a warm-blooded animal, especially a human.

4. A compound of formula (Ia) 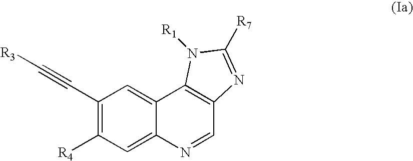
wherein
R₁ is substituted or unsubstituted phenyl where the phenyl is substituted with up to 4, preferably up to 2 substituents, wherein the substituents are the same or different and are independently selected from halo (e.g. Cl or F); cyano; cyano lower alkyl (e.g. cyanomethyl, cyanoethyl and cyanopropyl); lower alkyl; lower alkoxy; amino; amino-lower alkyl; amino-lower alkoxy; amino-lower alkyl sulfanyl or thiol-lower alkyl; wherein the amino group can be mono or disubstituted [e.g. —(C₁-C₇)NR⁸R₉ or —O—(C₁-C₇)NR₈R₉, wherein R₈ and R₉ can be the same or different and are independently H; lower alkyl (e.g. methyl, ethyl or propyl); lower cycloalkyl (e.g. cyclopropyl); or R₈ and R₉ together with the N atom form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl)]; amino-carbonyl-lower alkyl (e.g. R₈R₉—N—C(O)—CH₂—, wherein R₈ and R₉ are as defined above); heterocyclyl; heterocyclyl-lower alkyl; heterocyclyl-lower alkoxy or heterocyclyl-lower alkanesulfanyl wherein the heterocyclyl is a 3- to 8-membered heterocyclic ring containing 1-4 nitrogen, oxygen or sulfur atoms (e.g. imidazolyl, imidazolinyl, pyrrolidinyl, morpholinyl, azetidinyl, pyridyl, piperidino, piperidyl, piperazinyl or lower alkyl-piperazinyl); wherein alkyl may be linear or cyclic (e.g. cyclopropyl) and the alkyl in any of the substituents above may optionally be substituted with —NR₈R₉, wherein R₈ and R₉ are as defined above;
R₃ is hydrogen; lower alkyl; halo (e.g. fluoro, chloro or bromo); lower alkoxy (e.g. methoxy); or unsubstituted or substituted C₅-C₁₄aryl (e.g. phenyl, hydroxyphenyl, methoxyphenyl or aminosulfonyl-phenyl or benzo[1,3]dioxolo); or a heteroaryl being unsubstituted or substituted by one or more, especially 1-3 substituents; pyridyl (or an N-oxide of pyridyl) which is unsubstituted or substituted by one to two radicals selected from the group consisting of lower alkyl (e.g. methyl); lower alkoxy (e.g. methoxy); halo (e.g. fluoro); or —NR₈R₉, wherein R₈ and R₉ can be the same or different and are independently H; lower alkyl (e.g. methyl, ethyl or propyl); lower cycloalkyl (e.g. cyclopropyl); or the R₈ and R₉ can, with the N atom, form a 3 to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl);
R₄ is hydrogen or halo, especially fluoro; and
R₇ is hydrogen or an organic moiety, such as C₁-C₇lower alkyl; amino or amino lower alkyl; where alkyl may be unsubstituted or substituted with halo (e.g. methyl, ethyl, propyl, trifluoromethyl); lower alkoxy (e.g. methoxy); or cycloalkyl (e.g. cyclopropyl);
or a pharmaceutically acceptable salt thereof.

5. A compound of formula (Ib) 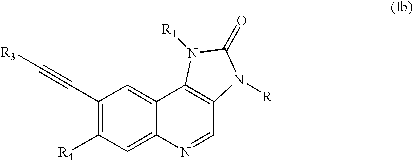
wherein
R₁ is substituted or unsubstituted phenyl where the phenyl is substituted with up to 4, preferably up to 2 substituents, wherein the substituents are the same or different and are independently selected from halo (e.g. Cl or F); cyano; cyano lower alkyl (e.g. cyanomethyl, cyanoethyl and cyanopropyl); lower alkyl; lower alkoxy; amino; amino-lower alkyl; amino-lower alkoxy; amino-lower alkyl sulfanyl or thiol-lower alkyl; wherein the amino group can be mono or disubstituted, [e.g. —(C₁-C₇)NR₈R₉ or —O—(C₁-C₇)NR₈R₉, wherein R₈ and R₉ can be the same or different and are independently H; lower alkyl (e.g. methyl, ethyl or propyl); lower cycloalkyl (e.g. cyclopropyl); or R₈ and R₉ together with the N atom form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl)]; amino-carbonyl-lower alkyl (e.g. R₈R₉—N—C(O)—CH₂—, wherein R₈ and R₉ are as defined above); heterocyclyl; heterocyclyl-lower alkyl; heterocyclyl-lower alkoxy or heterocyclyl-lower alkanesulfanyl wherein the heterocyclyl is a 3- to 8-membered heterocyclic ring containing 1-4 nitrogen, oxygen or sulfur atoms (e.g. imidazolyl, imidazolinyl, pyrrolidinyl, morpholinyl, azetidinyl, pyridyl, piperidino, piperidyl, piperazinyl or lower alkyl-piperazinyl); wherein alkyl may be linear or cyclic (e.g. cyclopropyl) and the alkyl in any of the substituents above may optionally be substituted with —NR₈R₉, wherein R₈ and R₉ are as defined above;
R₃ is hydrogen; lower alkyl; halo (e.g. fluoro, chloro or bromo); lower alkoxy (e.g. methoxy); or unsubstituted or substituted C₅-C₁₄aryl (e.g. phenyl, hydroxyphenyl, methoxyphenyl or aminosulfonyl-phenyl or benzo[1,3]dioxolo); or a heteroaryl being unsubstituted or substituted by one or more, especially 1-3, substituents; pyridyl (or an N-oxide of pyridyl) which is unsubstituted or substituted by one to two radicals selected from the group consisting of lower alkyl (e.g. methyl); lower alkoxy (e.g. methoxy); halo (e.g. fluoro); or —NR₈R₉, wherein R₈ and R₉ can be the same or different and are independently H; lower alkyl (e.g. methyl, ethyl or propyl); lower cycloalkyl (e.g. cyclopropyl); or the R₈ and R₉ can, with the N atom, form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl);
R₄ is hydrogen or halo, especially fluoro; and
R is hydrogen or substituted or unsubstituted C₁-C₇lower alkyl; amino; mono or disubstituted amino; lower alkoxy (e.g. OCH₃) or cycloalkyl (e.g. cyclopropyl);
or a pharmaceutically acceptable salt thereof.

6. Use of a compound of the formula (I) 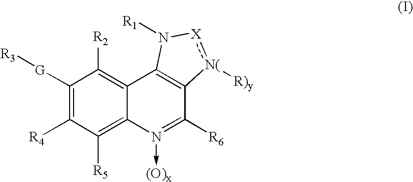
wherein
each of x and y is independently of the other 0 or 1;
R₁ is an organic moiety that can be bound to nitrogen;
X is C═O or C═S with the proviso that then the dashed line bonding X to N is absent, so that X is bound to the adjacent N via a single bond the with the proviso that then y is 1 and R is hydrogen or an organic moiety that can be bound to nitrogen; or
X is (CR₇), wherein R₇ is hydrogen or an organic or inorganic moiety with the proviso that then the dashed line bonding X to N is a bond, so that X is bound to the adjacent N via a double bond, and with the proviso that then y is zero or y is 1 and then —R is →O;
G is unsubstituted or substituted alkenylene, unsubstituted or substituted alkynylene; and
each of R₂, R₃, R₄, R₅ and R₆, independently of the others, is hydrogen, an organic moiety or an inorganic moiety;
or a pharmaceutically acceptable salt thereof for treating a protein kinase dependent disease.

7. A use according to claim 6, wherein the disease to be treated is a proliferative disease selected from a benign or malignant tumor, carcinoma of the brain, kidney, liver, adrenal gland, bladder, breast, stomach, gastric tumors, ovaries, colon, rectum, prostate, pancreas, lung, vagina or thyroid, sarcoma, glioblastomas, multiple myeloma or gastrointestinal cancer, especially colon carcinoma or colorectal adenoma, or a tumor of the neck and head, an epidermal hyperproliferation, psoriasis, prostate hyperplasia, a neoplasia, a neoplasia of epithelial character, a mammary carcinoma, a leukemia, Cowden syndrome, Lhermitte-Dudos disease or Bannayan-Zonana syndrome.

8. Use of a compound according to formula (I) of claim 1 in the preparation of a pharmaceutical composition.

9. A pharmaceutical composition comprising a compound according to claim 1.

10. A pharmaceutical composition comprising a compound according to claim 1 and a pharmaceutically acceptable carrier material.

11. A compound according to claim 1, selected from
2-[4-(8-Phenylethynyl-imidazo[4,5-c]quinolin-1-yl)-phenyl]-ethylamine;
2-{4-[8-(3-Methoxy-phenylethynyl)-imidazo[4,5-c]quinolin-1-yl]-phenyl}-ethylamine;
2-{4-[8-(4-Methoxy-phenylethynyl)-imidazo[4,5-c]quinolin-1-yl]-phenyl}-ethylamine;
2-[4-(8-Pyridin-3-ylethynyl-imidazo[4,5-c]quinolin-1-yl)-phenyl]-ethylamine;
2-{4-[8-(6-Methoxy-pyridin-3-ylethynyl)-imidazo[4,5-c]quinolin-1-yl]-phenyl}-ethylamine;
2-[4-(8-Benzo[1,3]dioxol-5-ylethynyl-imidazo[4,5-c]quinolin-1-yl)-phenyl]-ethylamine;
4-{1-[4-(2-Amino-ethyl)-phenyl]-1H-imidazo[4,5-c]quinolin-8-ylethynyl}-benzenesulfonamide;
3-[4-(8-Phenylethynyl-imidazo[4,5-c]quinolin-1-yl)-phenyl]-propylamine;
3-{4-[8-(4-Methoxy-phenylethynyl)imidazo[4,5-c]quinolin-1-yl]-phenyl}-propylamine;
3-{4-[8-(3-Methoxy-phenylethynyl)-imidazo[4,5-c]quinolin-1-yl]-phenyl}propylamine;
3-[4-(8-Pyridin-3-ylethynyl-imidazo[4,5-c]quinolin-1-yl)-phenyl]-propylamine;
3-[4-(8-Benzo[1,3]dioxol-5-ylethynyl-imidazo[4,5-c]quinolin-1-yl)-phenyl]-propylamine;
4-{1-[4-(3-Amino-propyl)-phenyl]-1H-imidazo[4,5-c]quinolin-8-ylethynyl}-benzenesulfonamide;
2-[4-(7-Chloro-8-phenylethynyl-imidazo[4,5-c]quinolin-1-yl)-phenyl]-ethylamine;
2-{4-[7-Chloro-8-(3-methoxy-phenylethynyl)-imidazo[4,5-c]quinolin-1-yl]-phenyl}-ethylamine;
2-{4-[7-Chloro-8-(4-methoxy-phenylethynyl)-imidazo[4,5-c]quinolin-1-yl]-phenyl}-ethylamine;
2-[4-(7-Chloro-8-pyridin-3-ylethynyl-imidazo[4,5-c]quinolin-1-yl)-phenyl]-ethylamine;
2-[4-(7-Chloro-8-benzo[1,3]dioxol-5-ylethynyl-imidazo[4,5-c]quinolin-1-yl)-phenyl]-ethylamine;
4-{1-[4-(2-Amino-ethyl)-phenyl]-7-chloro-1H-imidazo[4,5-c]quinolin-8-ylethynyl}-benzenesulfonamide;
3-[4-(7-Chloro-8-phenylethynyl-imidazo[4,5-c]quinolin-1-yl)-phenyl]-propylamine;
3-{4-[7-Chloro-8-(3-methoxy-phenylethynyl)-imidazo[4,5-c]quinolin-1-yl]-phenyl}-propylamine;
3-{4-[7-Chloro-8-(4-methoxy-phenylethynyl)-imidazo[4,5-c]quinolin-1-yl]-phenyl}-propylamine;
3-[4-(7-Chloro-8-pyridin-3-ylethynyl-imidazo[4,5-c]quinolin-1-yl)-phenyl]-propylamine;
3-[4-(7-Chloro-8-benzo[1,3]dioxol-5-ylethynyl-imidazo[4,5-c]quinolin-1-yl)-phenyl]-propylamine;
4-{1-[4-(3-Amino-propyl)-phenyl]-7-chloro-1H-imidazo[4,5-c]quinolin-8-ylethynyl}-benzenesulfonamide;
2-[4-(7-Fluoro-8-phenylethynyl-imidazo[4,5-c]quinolin-1-yl)-phenyl]-ethylamine;
2-{4-[7-Fluoro-8-(3-methoxy-phenylethynyl)-imidazo[4,5-c]quinolin-1-yl]-phenyl}-ethylamine;
2-{4-[7-Fluoro-8-(4-methoxy-phenylethynyl)-imidazo[4,5-c]quinolin-1-yl]-phenyl}-ethylamine;
2-[4-(7-Fluoro-8-pyridin-3-ylethynyl-imidazo[4,5-c]quinolin-1-yl)-phenyl]-ethylamine;
2-[4-(7-Fluoro-8-benzo[1,3]dioxol-5-ylethynyl-imidazo[4,5-c]quinolin-1-yl)-phenyl]-ethylamine;
4-{1-[4-(2-Amino-ethyl)-phenyl]-7-fluoro-1H-imidazo[4,5-c]quinolin-8-ylethynyl)benzenesulfonamide;
2-[4-(2-Methyl-8-phenylethynyl-imidazo[4,5-c]quinolin-1-yl)-phenyl]-ethylamine;
2-{4-[8-(3-Methoxy-phenylethynyl)-2-methyl-imidazo[4,5-c]quinolin-1-yl]-phenyl}-ethylamine;
2-{4-[8-(4-Methoxy-phenylethynyl)-2-methyl-imidazo[4,5-c]quinolin-1-yl]-phenyl}ethylamine;
2-[4-(2-Methyl-8-pyridin-3-ylethynyl-imidazo[4,5-c]quinolin-1-yl)-phenyl]-ethylamine;
2-[4-(2-Ethyl-8-pyridin-3-ylethynyl-imidazo[4,5-c]quinolin-1-yl)-phenyl]-ethylamine;
2-[4-(3-Propyl-8-pyridin-3-ylethynyl-imidazo[4,5-c]quinolin-1-yl)-phenyl]-ethylamine;
3-[4-(8-trans-Styryl-imidazo[4,5-c]quinolin-1-yl)-phenyl]-propylamine;
2-[4-(7-Chloro-8-styryl-imidazo[4,5-c]quinolin-1-yl)-phenyl]-ethylamine;
3-[4-(7-Chloro-8-styryl-imidazo[4,5-c]quinolin-1-yl)-phenyl]-propylamine;
2-{4-[8-(6-Fluoro-pyridin-3-ylethynyl)-imidazo[4,5-c]quinolin-1-yl]-phenyl}-ethylamine;
2-{4-[8-(6-Morpholin-4-yl-pyridin-3-ylethynyl)-imidazo[4,5-c]quinolin-1-yl]-phenyl}-ethylamine;
(5-{1-[4-(2-Amino-ethyl)-phenyl]-1H-imidazo[4,5-c]quinolin-8-ylethynyl}-pyridin-2-yl)-dimethyl-amine;
2-[4-(2-Methoxy-8-pyridin-3-ylethynyl-imidazo[4,5-c]quinolin-1-yl)-phenyl]-ethylamine;
2-[4-(2-Cyclopropyl-8-pyridin-3-ylethynyl-imidazo[4,5-c]quinolin-1-yl)-phenyl]-ethylamine;
2-[4-(2-Isopropyl-8-pyridin-3-ylethynyl-imidazo[4,5-c]quinolin-1-yl)-phenyl]-ethylamine;
Cyclopropyl-{2-[4-(8-pyridin-3-ylethynyl-imidazo[4,5-c]quinolin-1-yl)-phenyl]-ethyl}-amine;
Methyl-2-[4-(8-pyridin-3-ylethynyl-imidazo[4,5-c]quinolin-1-yl)-phenyl]-ethyl}-amine;
1-[4-(8-Pyridin-3-ylethynyl-imidazo[4,5-c]quinolin-1-yl)-phenyl]-piperidin-4-ylamine;
C-{1-[4-(8-Pyridin-3-ylethynyl-imidazo[4,5-c]quinolin-1-yl)-phenyl]-piperidinyl-4-yl}-methylamine;
2-[4-(2-Methoxy-8-pyridin-3-ylethynyl-imidazo[4,5-c]quinolin-1-yl)-phenyl]-ethylamine;
N-Methyl-C-[4-(8-pyridin-3-ylethynyl-imidazo[4,5-c]quinolin-1-yl)-phenyl]-methanesulfonamide;
1-[(2-Azetidin-1-yl-ethyl)-phenyl]-8-pyridin-3-ylethynyl-1H-imidazo[4,5-c]quinoline;
8-Pyridin-3-ylethynyl-1-[4-(2-pyrrolidin-1-yl-ethyl)-phenyl]-1H-imidazo[4,5-c]quinoline;
[3-Chloro-4-(8-pyridin-3-ylethynyl-imidazo[4,5-c]quinolin-1-yl)-phenyl]-acetonitrile;
[2-Chloro-4-(8-pyridin-3-ylethynyl-imidazo[4,5-c]quinolin-1-yl)-phenyl]-acetonitrile;
[3-Methyl-4-(8-pyridin-3-ylethynyl-imidazo[4,5-c]quinolin-1-yl)-phenyl]-acetonitrile;
[2-Methyl-4-(8-pyridin-3-ylethynyl-imidazo[4,5-c]quinolin-1-yl)-phenyl]-acetonitrile;
[3-(8-Pyridin-3-ylethynyl-imidazo[4,5-c]quinolin-1-yl)-phenyl]-acetonitrile;
Dimethyl-{2-[4-(8-pyridin-3-ylethynyl-imidazo[4,5-c]quinolin-1-yl)-phenyl]-ethyl}-amine;
Dimethyl-{2-[4-(2-methyl-8-pyridin-3-ylethynyl-imidazo[4,5-c]quinolin-1-yl)-phenyl]-ethyl}-amine;
{2-[4-(2-Methoxy-8-pyridin-3-ylethynyl-imidazo[4,5-c]quinolin-1-yl)-phenyl]-ethyl}-dimethyl-amine;
{1-[4-(2-Dimethylamino-ethyl)-phenyl]-8-pyridin-3-ylethynyl-1H-imidazo[4,5-c]quinolin-2-yl}-dimethyl-amine;
1-[4-(4-Methyl-piperazin-1-yl)-phenyl]-8-pyridin-3-ylethynyl-1H-imidazo[4,5-c]quinoline;
2-Methyl-1-[4-(4-methyl-piperazin-1-yl)-phenyl]-8-pyridin-3-ylethynyl-1H-imidazo[4,5-c]quinoline;
1-[4-(4-Methyl-piperazin-1-ylmethyl)-phenyl]-8-pyridin-3-ylethynyl-1H-imidazo[4,5-c]quinoline;
Dimethyl-{1-[4-(4-methyl-piperazin-1-ylmethyl)-phenyl]-8-pyridin-3-ylethynyl-1H-imidazo[4,5-c]quinolin-2-yl}amine;
1-[3-Fluoro-4-(4-methyl-piperazin-1-yl)-phenyl]-8-pyridin-3-ylethynyl-1H-imidazo[4,5-c]quinoline;
1-[3-Fluoro-4-(4-methyl-piperazin-1-yl)-phenyl]-2-methyl-8-pyridin-3-ylethynyl-1H-imidazo[4,5-c]quinoline;
1-[3-Fluoro-4-(4-methyl-piperazin-1-yl)-phenyl]-2-methoxy-8-pyridin-3-ylethynyl-1H-imidazo[4,5-]quinoline;
2-Methyl-1-(4-piperazin-1-yl-phenyl)-8-pyridin-3-ylethynyl-1H-imidazo[4,5-c]quinoline;
1-(3-Fluoro-4-piperazin-1-yl-phenyl)-2-methyl-8-pyridin-3-ylethynyl-1H-imidazo[4,5-c]quinoline;
2-(4-Methyl-piperazin-1-yl)-5-(8-pyridin-3-ylethynyl-imidazo[4,5-c]quinolin-1-yl)-benzonitrile;
2-(4-Methyl-piperazin-1-yl)-5-(2-methyl-8-pyridin-3-ylethynyl-imidazo[4,5-c]quinolin-1-yl)-benzonitrile;
5-(2-Methoxy-8-pyridin-3-ylethynyl-imidazo[4,5-c]quinolin-1-yl)-2-(4-methyl-piperazin-1-yl)-benzonitrile;
5-(2-Dimethylamino-8-pyridin-3-ylethynyl-imidazo[4,5-c]quinolin-1-yl)-2-(4-methyl-piperazin-1-yl)-benzonitrile;
2-piperazin-1-yl-5-(8-pyridin-3-ylethynyl-imidazo[4,5-c]quinolin-1-yl)-benzonitrile;
5-(2-Methyl-8-pyridin-3-ylethynyl-imidazo[4,5-c]quinolin-1-yl)-2-piperazin-1-yl-benzonitrile;
5-(2-Methoxy-8-pyridin-3-ylethynyl-imidazo[4,5-c]quinolin-1-yl)-2-piperazin-1-yl-benzonitrile;
5-(2-Dimethylamino-8-pyridin-3-ylethynyl-imidazo[4,5-c]quinolin-1-yl)-2-piperazin-1-yl-benzonitrile;
3-(8-Pyridin-3-ylethynyl-imidazo[4,5-c]quinolin-1-yl)-benzonitrile;
3-(2-Methyl-8-pyridin-3-ylethynyl-imidazo[4,5-c]quinolin-1-yl)-benzonitrile;
3-(2-Methoxy-8-pyridin-3-ylethynyl-imidazo[4,5-c]quinolin-1-yl)benzonitrile;
3-(2-Dimethylamino-8-pyridin-3-ylethynyl-imidazo[4,5-c]quinolin-1-yl)-benzonitrile;
4-(8-Pyridin-3-ylethynyl-imidazo[4,5-c]quinolin-1-yl)-benzonitrile;
4-(2-Methyl-8-pyridin-3-ylethynyl-imidazo[4,5-c]quinolin-1-yl)-benzonitrile;
4-(2-Methoxy-8-pyridin-3-ylethynyl-imidazo[4,5-c]quinolin-1-yl)-benzonitrile;
4-(2-Dimethylamino-8-pyridin-3-ylethynyl-imidazo[4,5-c]quinolin-1-yl)-benzonitrile;
[4-(8-Pyridin-3-ylethynyl-imidazo[4,5-c]quinolin-1-yl)-phenyl]-acetonitrile;
[4-(2-Methyl-8-pyridin-3-ylethynyl-imidazo[4,5-c]quinolin-1-yl)-phenyl]-acetonitrile;
[4-(2-Ethyl-8-pyridin-3-ylethynyl-imidazo[4,5-c]quinolin-1-yl)-phenyl]-acetonitrile;
[4-(2-Methoxy-8-pyridin-3-ylethynyl-imidazo[4,5-c]quinolin-1-yl)-phenyl]-acetonitrile;
[4-(2-Dimethylamino-8-pyridin-3-ylethynyl-imidazo[4,5-c]quinolin-1-yl)-phenyl]-acetonitrile;
{4-[2-(3-Dimethylamino-propyl)-8-pyridin-3-ylethynyl-imidazo[4,5-c]quinolin-1-yl]-phenyl}acetonitrile;
{4-[8-(6-Morpholin-4-yl-pyridin-3-ylethynyl)-imidazo[4,5-c]quinolin-1-yl]-phenyl}acetonitrile;
{-[8-(1-Oxy-pyridin-3-ylethynyl)-imidazo[4,5-c]quinolin-1-yl]-phenyl}-acetonitrile;
[4-(4-Amino-8-pyridin-3-ylethynyl-imidazo[4,5-c]quinolin-1-yl)-phenyl]-acetonitrile;
[4-(4-Methylamino-8-pyridin-3-ylethynyl-imidazo[4,5-c]quinolin-1-yl)-phenyl]-acetonitrile;
[2-Fluoro-4-(8-pyridin-3-ylethynyl-imidazo[4,5-c]quinolin-1-yl)-phenyl]-acetonitrile;
[2-Fluoro-4-(2-methyl-8-pyridin-3-ylethynyl-imidazo[4,5-c]quinolin-1-yl)-phenyl]-acetonitrile;
[2-Fluoro-4-(2-methoxy-8-pyridin-3-ylethynyl-imidazo[4,5-c]quinolin-1-yl)-phenyl]-acetonitrile;
[4-(2-Dimethylamino-8-pyridin-3-ylethynyl-imidazo[4,5-c]quinolin-1-yl)-2-fluoro-phenyl]-acetonitrile;
2-Methyl-2-[4-(8-pyridin-3-ylethynyl-imidazo[4,5-c]quinolin-1-yl)-phenyl]-propionitrile;
2-Methyl-2-[4-(2-methyl-8-pyridin-3-ylethynyl-imidazo[4,5-c]quinolin-1-yl)-phenyl]-propionitrile;
2-[4-(2-Methoxy-8-pyridin-3-ylethynyl-imidazo[4,5-c]quinolin-1-yl)-phenyl]-2-methyl-propionitrile;
2-[2-Fluoro-4-(8-pyridin-3-ylethynyl-imidazo[4,5-c]quinolin-1-yl)-phenyl]-2-methyl-propionitrile;
2-[2-Fluoro-4-(2-methyl-8-pyridin-3-ylethynyl-imidazo[4,5-c]quinolin-1-yl)-phenyl]-2-methyl-propionitrile;
2-[4-(2-Dimethylamino-8-pyridin-3-ylethynyl-imidazo[4,5-c]quinolin-1-yl)-2-fluoro-phenyl]-2-methyl-propionitrile;
3-[4-(8-Pyridin-3-ylethynyl-imidazo[4,5-c]quinolin-1-yl)-phenyl]-propionitrile;
3-[4-(2-Methyl-8-pyridin-3-ylethynyl-imidazo[4,5-c]quinolin-1-yl)-phenyl]-propionitrile;
3-[4-(2-Methoxy-8-pyridin-3-ylethynyl-imidazo[4,5-c]quinolin-1-yl)-phenyl]-propionitrile;
3-[4-(2-Dimethylamino-8-pyridin-3-ylethynyl-imidazo[4,5-c]quinolin-1-yl)-phenyl]-propionitrile;
1-[2-Fluoro-4-(8-pyridin-3-ylethynyl-imidazo[4,5-c]quinolin-1-yl)-phenyl]-pyrrolidin-2-one;
1-[2-Fluoro-4-(2-methyl-8-pyridin-3-ylethynyl-imidazo[4,5-c]quinolin-1-yl)-phenyl]-pyrrolidin-2-one;
1-[2-Fluoro-4-(2-methoxy-8-pyridin-3-ylethynyl-imidazo[4,5-c]quinolin-1-yl)-phenyl]-pyrrolidin-2-one;
1-[4-(2-Dimethylamino-8-pyridin-3-ylethynyl-imidazo[4,5-c]quinolin-1-yl)-2-fluoro-phenyl]-pyrrolidin-2-one;
1-[4-(8-Pyridin-3-ylethynyl-imidazo[4,5-c]quinolin-1-yl)-phenyl]-pyrrolidin-2-one;
1-[4-(2-Methyl-8-pyridin-3-ylethynyl-imidazo[4,5-c]quinolin-1-yl)-phenyl]-pyrrolidin-2-one;
1-[4-(2-Methoxy-8-pyridin-3-ylethynyl-imidazo[4,5-c]quinolin-1-yl)-phenyl]-pyrrolidin-2-one;
1-[4-(2-Dimethylamino-8-pyridin-3-ylethynyl-imidazo[4,5-c]quinolin-1-yl)-phenyl]-pyrrolidin-2-one;
2-(2-Oxo-pyrrolidin-1-yl)-5-(8-pyridin-3-ylethynyl-imidazo[4,5-c]quinolin-1-yl)-benzonitrile;
5-(2-Methyl-8-pyridin-3-ylethynyl-imidazo[4,5-c]quinolin-1-yl)-2-(2-oxo-pyrrolidin-1-yl)benzonitrile;
5-(2-Methoxy-8-pyridin-3-ylethynyl-imidazo[4,5-c]quinolin-1-yl)-2-(2-oxo-pyrrolidin-1-yl)-benzonitrile;
5-(2-Dimethylamino-8-pyridin-3-ylethynyl-imidazo[4,5-c]quinolin-1-yl)-2-(2-oxo-pyrrolidin-1-yl)-benzonitrile;
3-[2-Fluoro-4-(8-pyridin-3-ylethynyl-imidazo[4,5-c]quinolin-1-yl)-phenyl]-oxazolidin-2-one;
3-[2-Fluoro-4-(2-methyl-8-pyridin-3-ylethynyl-imidazo[4,5-c]quinolin-1-yl)-phenyl]-oxazolidin-2-one;
3-[2-Fluoro-4-(2-methoxy-8-pyridin-3-ylethynyl-imidazo[4,5-c]quinolin-1-yl)-phenyl]-oxazolidin-2-one;
3-[4-(2-Dimethylamino-8-pyridin-3-ylethynyl-imidazo[4,5-c]quinolin-1-yl)-2-fluoro-phenyl]-oxazolidin-2-one;
3-[4-(8-Pyridin-3-ylethynyl-imidazo[4,5-c]quinolin-1-yl)-phenyl]-oxazolidin-2-one;
3-[4-(2-Methyl-8-pyridin-3-ylethynyl-imidazo[4,5-c]quinolin-1-yl)-phenyl]-oxazolidin-2-one;
3-[4-(2-Methoxy-8-pyridin-3-ylethynyl-imidazo[4,5-c]quinolin-1-yl)-phenyl]-oxazolidin-2-one;
1-[2-Fluoro-4-(8-pyridin-3-ylethynyl-imidazo[4,5-c]quinolin-1-yl)-phenyl]-pyrrolidine-2,5-dione;
1-[2-Fluoro-4-(2-methyl-8-pyridin-3-ylethynyl-imidazo[4,5-c]quinolin-1-yl)-phenyl]-pyrrolidine-2,5-dione;
1-[2-Fluoro-4-(2-methoxy-8-pyridin-3-ylethynyl-imidazo[4,5-c]quinolin-1-yl)-phenyl]-pyrrolidine-2,5-dione;
1-[4-(2-Dimethylamino-8-pyridin-3-ylethynyl-imidazo[4,5-c]quinolin-1-yl)-2-fluoro-phenyl]-pyrrolidine-2,5-dione;
1-[2-Fluoro-4-(8-pyridin-3-ylethynyl-imidazo[4,5-c]quinolin-1-yl)-phenyl]-pyrrolidine-2,5-dione;
1-[2-Fluoro-4-(2-methyl-8-pyridin-3-ylethynyl-imidazo[4,5-c]quinolin-1-yl)-phenyl]-pyrrolidine-2,5-dione;
1-[2-Fluoro-4-(2-methoxy-8-pyridin-3-ylethynyl-imidazo-[4,5-c]quinolin-1-yl)-phenyl]-pyrrolidine-2,5-dione;
4-[2-Fluoro-4-(8-pyridin-3-ylethynyl-imidazo[4,5-c]quinolin-1-yl)-phenyl]-piperazin-2-one;
1-Ethyl-4-[2-fluoro-4-(8-pyridin-3-ylethynyl-imidazo[4,5-c]quinolin-1-yl)-phenyl]-piperazin-2-one;
1-Ethyl-4-[2-fluoro-4-(8-pyridin-3-ylethynyl-imidazo[4,5-c]quinolin-1-yl)-phenyl]-piperazin-2-one;
1-Ethyl-4-[2-fluoro-4-(2-methyl-8-pyridin-3-ylethynyl-imidazo[4,5-c]quinolin-1-yl)-phenyl]-piperazin-2-one;
4-[2-Fluoro-(8-pyridin-3-ylethynyl-imidazo[4,5-c]quinolin-1-yl)-phenyl]-1-methyl-piperazin-2-one;
4-[2-Fluoro-4-(2-methyl-8-pyridin-3-ylethynyl-imidazo[4,5-c]quinolin-1-yl)-phenyl]-1-methyl-piperazin-2-one;
2-Cyanomethyl-5-(2-methyl-8-pyridin-3-ylethynyl-imidazo[4,5-c]quinolin-1-yl)-benzonitrile;
2-(Cyano-dimethyl-methyl)-5-(2-methyl-8-pyridin-3-ylethynyl-imidazo[4,5-c]quinolin-1-yl)-benzonitrile;
1-(4-Fluoro-phenyl)-3-methyl-8-pyridin-3-ylethynyl-1,3-dihydro-imidazo[4,5-c]quinolin-2-one;
1-(4-Ethyl-phenyl)-3-methyl-8-pyridin-3-ylethynyl-1,3-dihydro-imidazo[4,5-c]quinolin-2-one;
1-(3-Methoxy-phenyl)-3-methyl-8-pyridin-3-ylethynyl-1,3-dihydro-imidazo[4,5-c]quinolin-2-one;
1-(4-ethoxy-phenyl)-3-methyl-8-pyridin-3-ylethynyl-1,3-dihydro-imidazo[4,5-c]quinolin-2-one;
3-Methyl-8-pyridin-3-ylethynyl-1-(3,4,5-trimethoxy-phenyl)-1,3-dihydro-imidazo[4,5-c]quinolin-2-one;
2-Methyl-2-[4-(3-methyl-2-oxo-8-pyridin-3-ylethynyl-2,3-dihydro-imidazo[4,5-c]quinolin-1-yl)-phenyl]-propionitrile;
3-Methyl-1-[4-(2-oxo-oxazolidin-3-yl)-phenyl]-8-pyridin-3-ylethynyl-1,3-dihydro-imidazo[4,5-c]quinolin-2-one;
1-[3-Fluoro-4-(2-oxo-oxazolidin-3-yl)-phenyl]-3-methyl-8-pyridin-3-ylethynyl-1,3-dihydro-imidazo[4,5-c]quinolin-2-one;
3-Methyl-1-(4-piperazin-1-yl-phenyl)-8-pyridin-3-ylethynyl-1,3-dihydro-imidazo[4,5-c]quinolin-2-one;
1-(3-Fluoro-4-piperazin-1-yl-phenyl)-3-methyl-8-pyridin-3-ylethynyl-1,3-dihydro-imidazo[4,5-c]quinolin-2-one;
3-Methyl-1-(4-methylamino-phenyl)-8-pyridin-3-ylethynyl-1,3-dihydro-imidazo[4,5-c]quinolin-2-one;
N-Methyl-N-[4-(3-methyl-2-oxo-8-pyridin-3-ylethynyl-2,3-dihydro-imidazo[4,5-c]quinolin-1-yl)-phenyl]-acetamide; and pharmaceutically acceptable salts thereof.

12. A process to prepare a compound according to claim 1, comprising reacting a compound of the formula (IIa) 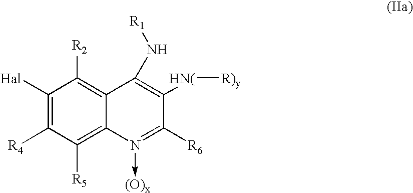
with an alkenylene or alkynylene derivative;
and x, y, X, R₁, R₂, R₄, R₅, R₆ and R, are as defined in claim 1;
and, if desired, transforming an obtainable compound of formula (I) into a different compound of formula (I), transforming a salt of an obtainable compound of formula (I) into the free compound or a different salt, or an obtainable free compound of formula (I) into a salt; and/or separating an obtainable mixture of isomers of compounds of formula (I) into the individual isomers.
